(19) United States
(12) Patent Application Publication
(10) Pub. No.: US 2021/0386812 A1
(43) Pub. Date: Dec. 16, 2021

(54) DEVELOPMENT OF HEALTH FOOD SUPPLEMENTS AND ANTIOXIDANTS FOR CONTROLLING HYPERURICEMIA AND OXIDATIVE STRESS

(71) Applicant: Baylor College of Medicine, Houston, TX (US)

(72) Inventors: Changyi Chen, Houston, TX (US); Qizhi Yao, Houston, TX (US); Jian-Ming Lu, Pearland, TX (US)

(21) Appl. No.: 16/972,950

(22) PCT Filed: Jun. 13, 2019
(86) PCT No.: PCT/IB2019/000801

§ 371 Date: Dec. 7, 2020

Related U.S. Application Data
(60) Provisional application No. 62/684,406, filed on Jun. 13, 2018.

(51) Int. Cl. A61K 36/534; A61K 36/28; A61K 31/519; A61K 31/593; A61K 31/426; A23L 33/105; A23L 33/155

(52) U.S. Cl. CPC A61K 36/534; A61K 36/28; A61K 31/519; A61K 2236/53; A61K 31/426; A23L 33/105; A23L 33/155; A61K 31/593

(57) ABSTRACT

Embodiments of the disclosure include methods and compositions that comprise one or more herb extracts and optionally folic acid and/or one or more of its derivatives. In specific embodiments, the disclosure concerns treatment or prevention of hyperuricemic conditions with combinatorial compositions, such as those including one or more Chinese herbal medicine extracts and optionally folic acid and/or one or more of its derivatives.

This application claims priority to U.S. Provisional Patent Application Ser. No. 62/684,406, filed Jun. 13, 2018, which is incorporated by reference herein in its entirety.

### TECHNICAL FIELD

Embodiments of the disclosure encompass at least the fields of cell biology, molecular biology, and medicine.

### BACKGROUND

Based on the 2018 report from the American Heart Association (1), there are 836,546 deaths caused by cardiovascular disease (CVD) in the United States in 2015; it accounts about 2,300 Americans who died from CVD each day, an average of 1 death every 38 seconds. CVD still remains the number one cause of death in the United States. About 92.1 million American adults are living with some form of CVD. In addition to several traditional CVD risk factors, there are many new risk factors, which have recently been recognized and further investigated. For example, hyperuricemia has long been established as the major etiologic factor in gout. Besides gout, the association of hyperuricemia with CVD was described 2 centuries ago (2). Only in the last few years, several large clinical studies have confirmed that hyperuricemia is a significant and independent risk factor for CVD including ischemic heart disease, heart failure and hypertension after an extensive adjustment for almost all of the possible confounding conditions (3-8). Hyperuricemia is an independent risk factor of atrial fibrillation (9). High levels of serum uric acid (SUA) directly impair the cardiovascular system in a concentration-dependent matter. For each increase of 1 mg/dL of SUA, the overall risks of coronary heart disease and all-cause mortality increased by 20 and 9%, respectively (3-8). Increase in SUA level by 1 mg/dl resulted in an equivalent risk of 20 mg/dl increase in serum cholesterol as well as 10 mm Hg elevation of systolic blood pressure (10). SUA levels increased by 1 mg/dL, the risk of hypertension increased by 15% to 23% (11-14). The risk of type 2 diabetes mellitus was also increased by 6% for every 1 mg/dL increase (15).

The prevalence of gout and hyperuricemia in the USA is about 4% and 21%, respectively (16). The pooled prevalence of gout and hyperuricemia in mainland China from 2000 to 2014 was 1.1% and 13-25%, respectively (17,18). In the some areas of the world, the prevalence of gout and hyperuricemia is high, up to 11.7% and 41.1, respectively (19). Xanthine oxidase (XO) is a rate-limiting enzyme in purine catabolism, generating final product, uric acid; during the chemical reaction, XO also produces reactive oxygen species (superoxide anion and H₂O₂). Thus, both hyperuricemia and oxidative stress generated from XO significantly contributes to the development of CVD. Accordingly, urate-lowering drugs such as XO-inhibitors could play an important role in the prevention and treatment of CVD. Thus, SUA is a central player, not an innocent bystander, in CVD and several other diseases. Mechanistically, hyperuricemia contributes to the progression of CVD through oxidative stress, systemic inflammation, and endothelial dysfunction (20). Clinically, treatment with Allopurinol, a XO-inhibitor drug, significantly reduced the risk of myocardial infarction, reduced all-cause and cardiovascular mortality in high-risk patients, and improved endothelial functions in several clinical trials (2,20-24). However, because of its potential adverse effects, clinically available XO-inhibitor drug, Allopurinol, is not indicated for clinical management of chronic hyperuricemia-related CVD and other diseases including diabetes (26), insulin resistance (27), metabolic syndrome (28), chronic kidney disease (29), psoriatic arthritis (30), micro-albuminuria (31), erectile dysfunction (32), preeclampsia (33), cancers (34,35), immune disorders (36), ischemia reperfusion injury (37), inflammatory diseases (38,39), or tumor lysis syndrome. Febuxostat is another FDA-approved XO-inhibitor drug; however, it also has potential side effects, which prevent it from long-term administration for asymptomatic hyperuricemia.

In the clinical trials, the 10 most common treatment-related adverse events in LTE studies by preferred term during febuxostat treatment (regardless of dose) were: hepatic enzyme increased, ALT increased, liver function test abnormal, AST increased, hyperlipidaemia, blood creatinine increased, nephrolithiasis, arthritis, blood urea increased, and gamma-glutamyltransferase (GGT) increased (40). A recent report showed a clinical case of febuxostat-induced liver injury (41). Febuxostat cannot be recommended for patients with moderate or severe hepatic impairment. The most common adverse effect leading to discontinuation of febuxostat was elevated liver function tests: in some studies up to 2-3% of patients developed transaminase elevations greater than three times the upper limit of normal. However, these studies did not establish a dose-effect relationship between febuxostat and elevated liver function tests (42).

In the first trials, cardiovascular events were a concern related to febuxostat treatment, especially concerning thromboembolic events, myocardial infarcts, and strokes (43). This is initial evidence of nonfatal cardiovascular events as febuxostat side effects, leading the FDA to require a long-term cardiovascular study as a condition for approving the drug.

In addition, initial clinical studies showed that febuxostat can also lead to cutaneous adverse effects in about 2% of patients (44-47). Cases of severe febuxostat hypersensitivity reactions such as SJS and anaphylactic shock are reported (46,48). These serious adverse effects with febuxostat are potentially associated with a history of skin reaction to allopurinol, particularly in patients with renal failure (46,49,50). A case report also showed that febuxostat induced rhabdomyolysis (51). Thus, febuxostat is currently not recommended for the treatment of asymptomatic hyperuricemia.

Currently, the development of newer agents with differing pharmacological mechanisms and less toxicity is an active field of research.

Additionally, there is an active research on the development of alternative medicines such as functional food/dietary health supplements and herb medicines for the long-term control of hyperuricemia with no or much less adverse effects compared to current XO-inhibitor drugs. Certain commercially-available dietary supplements might be beneficial in lowering uric acid levels in the body, but not all supplements used for this purpose are backed by extensive scientific research studies.

### BRIEF SUMMARY

Embodiments of the disclosure include methods and compositions for the treatment of one or more medical conditions. In specific embodiments, the medical condition is one in which inhibition of Xanthine oxidase (XO) activity would be therapeutically effective, such as to reduce the presence, severity, or onset of at least one symptom of the medical condition.

The present disclosure concerns the development of alternative medicines, and in an initial study there was testing of XO inhibitory effects of 72 traditional Chinese herb medicines. The dynamic effects of folic acid on XO inhibition was examined, because folic acid is one of the most commonly used dietary supplements. Folic acid and its derivatives have been demonstrated to have XO inhibitory effects (52,53).

In particular embodiments, some traditional Chinese herb medicines have potent XO-inhibitory activities; individual herb activities may have additive or synergistic effects with other herb medicines and/or with folic acid and/or one or more of its derivatives. Folic acid and its derivative 5-methyl tetrahydrofolate (5-MTHF) are XO inhibitors (52,53). Recent clinical trials have shown that folic acid supplement reduces SUA levels in hypertensive patients and reduced the risk for CVD (54-59). The functional food/dietary supplement formulation of the present disclosure that includes the use of traditional Chinese herb extracts and/or folic acid and/or one or more of its derivatives provides a new opportunity to prevent and/or treat a variety of medical conditions associated with XO, such as asymptomatic hyperuricemia, for example. Given the high prevalence of asymptomatic hyperuricemia (about 20% in general population in China and US) and lack of safe XO-inhibitor drugs, this novel approach is useful for its long-term management of asymptomatic hyperuricemia. Given that asymptomatic hyperuricemia is an independent risk factor for CVD and many other diseases, the disclosed methods have enormous impact on disease prevention.

In particular embodiments, the compositions comprising one or more herb extracts are considered non-natural, because extracts are not found in nature.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows XO inhibitory effects of 72 traditional Chinese herb medicines (standard extracts) in a cell-free system. Each extract was studied in both water-soluble (blue (left of a pair)) and dimethyl sulfoxide (DMSO)-soluble (red (right of a pair)) fractions. Final concentration of each extract is 163 ug/ml). Blue bar: DMSO-soluble fraction; Red bar: water-soluble fraction of each extract.

FIGS. 2A-2N demonstrate dose-dependent effects of 14 standard herb extracts on XO inhibition in the cell free system. DMSO-soluble fractions of these extracts were used in this assay.

FIG. 3 shows XO inhibitory effects of folic acid and its two derivatives, dihydrofolic acid and tetrahydrofolic acid, in a cell-free system.

FIG. 4 provides comparison of folic acid, allopurinol and 3,4-Dihydroxy-5-nitrobenzaldehyde (DHNB) on XO inhibition in a cell-free system.

FIG. 5 illustrates the chemical structure of folic acid, dihydrofolic acid (DHF), tetrahydrofolic acid (THF), 5,10-methylenetetrahydrofolate (5,10-MTHFR), 5-methyl tetrahydrofolate (5-MTHF), 10-Formyl tetrahydrofolate (10-FTHF), and 6-formylpterin.

FIG. 6. illustrates that folic acid is a critical vitamin, which have many important biological functions in the body, including DNA and RNA synthesis, methylation of DNA phospholipid, and proteins for functional regulation, homocysteine metabolism for reducing its toxicity and for methionine synthesis for multiple functions. Serine hydroxymethyltransferase (SHMT); Dihydrofolate Reductase (DHFR); Thymidylate synthase (TS); Methylenetetrahydrofolate reductase (MTHFR); Methionine Synthase (MS); S-adenosyl-methionine (SAM).

FIG. 7 shows a combination of Chinese mint leave extract with folic acid at relatively small concentrations for XO inhibitory assay. The result shows an additive effect of Chinese mint leave extract with folic acid for XO inhibition. Chinese mint leave extract and folic acid are separately added into XO reaction system, without pre-mixing.

FIGS. 8A-8N show the effect of combination of traditional Chinese medicine herb extracts and folic acid on XO inhibition. All 14 traditional Chinese medicine herb extracts were tested. Folic acid (0.167 uM) and extract (13.3 ug/ml) were separately added into the XO reaction system without pre-mixing. XO activity was measured.

FIG. 9 provides the effect of 5-methyl tetrahydrofolate (5-MTHF) on XO inhibition. Fresh prepared 5-MTHF inhibited XO activities in a concentration-dependent manner. IC₅₀ for 5-MTHF is about 35.6 uM. However, its effect is weaker than folic acid.

FIGS. 10A-10N demonstrate the effect of combination of 5-MTHF and herb extracts on XO inhibition in vitro. Fresh prepared 5-MTHF (3.3 uM) and herb extract (13.3 ug/ml) was added to XO reaction system separately, XO activity was recorded. 5-MTHF had an additive effect on XO inhibition with 10 out of 14 herb extracts: Flowering Quince (Fruit) Extract; Euryale (Seed) Extract; Fringed Pink (Aboveground Parts) Extract, Honeysuckle (Flower Bud) Extract, Houttuynia (Aboveground Parts); Citron (Fruit) Extract; Sacred Lotus (Seed) Extract; Lesser Galangal Extract; Wild Chrysanthemum (Flower) Extract; and Chinese mint (FIG. 10), but not with Clove (Flower Bud) Extract; Vietnamese Sophora (Root) Extract; Rice Bean Extract; and Wheat (Immature Fruit) Extract.

FIG. 11 shows XO inhibitory effects of 112 herbal extracts in a cell-free system. Each extract was studied in a DMSO-soluble fraction. Final concentration of each extract is 166.7 ug/ml) for the majority of extracts; a few extracts at lower concentrations were used due to the solubility issue.

FIGS. 12A-12O demonstrate dose-dependent effects of 15 standard herb extracts on XO inhibition in the cell free system. DMSO-soluble fractions of these extracts were used in this assay.

FIGS. 13A-13H provide an effect of combination of selected herbal extracts and folic acid on XO inhibition. Eight herbal extracts were selected and tested. Folic acid (0.167 uM) and each extract at a defined concentration (ug/ml) were separately added into the XO reaction system without pre-mixing. XO activity was measured.

FIG. 14 shows an effect of rationally-designed dietary supplement recipe on reduction of serum uric acid levels in a mouse model. Recipe includes Japanese Thistle Extract, Grape Seed Extract, Amla Fruit Extract, Pine Bark Extract, Chinese Mint Extract and Folic acid. Mice were Intraperitoneally (i.p.)-injected with uricase inhibitor allantoxanamide to induce hyperuricemia in mice. The mice were then oral-gavaged with 200 ul to 250 ul (for 20 to 25 g mice) allopurinol (positive control) or dietary health supplement recipe (low dose and high dose). Blood was taken from facial vein at 1.5 hour and 3 hour after the treatment. Serum uric acid level was measured the next day with a phosphotunstate method. N=4 per group.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the subject matter may “consist essentially of” or “consist of” one or more elements or steps of the subject matter, for example. Some embodiments of the subject matter may consist of or consist essentially of one or more elements, method steps, and/or methods of the subject matter. It is contemplated that any method or composition described herein can be implemented with respect to any other method or composition described herein.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to excessive uric acid levels, for example. For subjects having or suspected of having a medical condition directly or indirectly associated with excessive uric acid levels, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The compositions of the present disclosure may be suitable for the treatment of diseases in a human or animal patient. In one embodiment, the patient is a mammal including a human, horse, dog, cat, sheep, cow, or primate. In one embodiment the patient is a human. In a further embodiment, the patient is not a human. The individual may be receiving one or more compounds through the world wide web.

As used herein, the term “effective amount” means that amount of a compound, drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “treatment” refers to defending against or inhibiting a symptom, treating a symptom, delaying the appearance of a symptom, reducing the severity of the development of a symptom, and/or reducing the number or type of symptoms suffered by an individual, as compared to not administering a pharmaceutical composition of the disclosure. The term treatment encompasses the use in a palliative setting

Embodiments of the disclosure include one or more compositions that encompass one or more herb extracts and optionally folic acid and/or one or more derivatives. Any of the elements listed herein may be combined with any one or more other elements as part of a composition. One or more components within any composition may impart an additive or synergistic effect. In specific embodiments, the composition(s) are utilized for treating or preventing a variety of medical conditions, including those in which it would be beneficial to reduce xanthine oxidase (XO) activity. Examples of medical conditions include one or more of direct or indirect hyperuricemic conditions, such as gout, hypertension, atherosclerosis, coronary artery disease, heart failure, left ventricular hypertrophy, atrial fibrillation, periphery artery disease, vascular restenosis, vascular thrombosis, stroke, diabetes, insulin resistance, metabolic syndrome, chronic kidney disease, psoriatic arthritis, micro-albuminuria, erectile dysfunction, preeclampsia, cancer (of any kind), immune disorders, or an inflammatory disease. When the medical condition is cancer, it may be of the lung, breast, brain, liver, colon, skin, stomach, prostate, uterus, endometrium, ovary, testes, bone, spleen, thyroid, blood, gall bladder, kidney, and so forth.

In some embodiments, there is a composition comprising Euryale (Seed) Extract; Rice Bean Extract; Wheat (Immature Fruit) Extract; Houttuynia (Aboveground Parts) Extract; Citron (Fruit) Extract; Sacred Lotus (Seed) Extract; Chinese Mint Extract; Fennel (Fruit) Extract; Canadian Thistle (Aboveground Parts) Extract; White Hyacinth Bean Extract; Sea-ear Shell Extract; Fructus Hordei Germinatus Extract; Sterculia (Seed) Extract; Haizhou Elsholtzia (Aboveground Parts) Extract; Sharp-Leaf Galangal (Seed) Extract; Radish (Seed) Extract; Sacred Lotus (Plumule) Extract; Fermented Soybean Extract; Jujube (Seed) Extract; Butcher's Broom; Oolong Tea (leaf) Extract; Senna Leaf Extract; Yohimbe bark Extract; Japanese Thistle extract; Butterbur Extract; Caralluma bark (wild) Extract; Echinacea root Extract; Horse Chestnut fruit Extract; Kola Nut Extract; Magnolia Bark Extract; Muira Puama Bark Extract; or a combination thereof. In such cases, the composition may be formulated as a pharmaceutical, medicinal food, supplement, food, food supplement, nutritional or dietary supplement, food for special dietary use, or medical food, or combination thereof.

Any composition of the disclosure may include one or more of Clove (Flower Bud) Extract; Vietnamese Sophora (Root) Extract; Flowering Quince (Fruit) Extract; Fringed Pink (Aboveground Parts) Extract; Honeysuckle (Flower Bud) Extract; Lesser Galangal Extract (Alpinia officinarum); or Wild Chrysanthemum (Flower) Extract; Fructose Crataegi (Charred) Extract; Chinese Licorice (Root & Rhizome) Extract; Ginkgo Leaf Extract; Sickle-pod Senna (Seed) Extract; Fructus Aurantii Processed Extract; Bitter Orange (Young Fruit) Extract; Chinese Smilax (Rhizome) Extract; White Mulberry (Young Twig) Extract; White Mulberry (Leaf) Extract; Sacred Lotus (Leaf) Extract; Chrysanthemum (Flower) Extract; Perilla (Leaf) Extract; Job's Tears Extract; Palm-leaf Raspberry Extract; or Patchouli (Aboveground Parts) Extract; African Mango seed Extract; Amla Fruit Powder; Grape Seed Extract; Green Tea Pure Extract; Milk Thistle Extract; Olive Leaf Extract; Pine Bark; Pomegranate Extract; Spearmint Leaf Extract; St John's Wort Extract; Aloe Vera Leaf Extract; Catuaba bark Bark Extract; Gynostemma Extract; Hawthorn Leaf Extract; Kudzu Root Extract; Lemon Balm Extract; Licorice Root Extract; Moringa Extract; Papaya Fruit Extract; Papaya Seed; Red Clover (steam-leaf) Extract; Rosemary Extract; or a combination thereof.

In particular embodiments, the composition comprises Euryale (Seed) Extract; Rice Bean Extract; Wheat (Immature Fruit) Extract; Houttuynia (Aboveground Parts) Extract; Citron (Fruit) Extract; Sacred Lotus (Seed) Extract; Chinese Mint Extract; Butcher's Broom Extract; Oolong Tea (leaf) Extract; Senna Leaf Extract; Yohimbe bark Extract; Japanese Thistle extract; Butterbur Extract; Caralluma bark (wild) Extract; Echinacea root Extract; Horse Chestnut fruit Extract; Kola Nut Extract; Magnolia Bark Extract; Muira Puama Bark Extract; or a combination thereof. In specific cases, the composition comprises Euryale (Seed) Extract and one or more other compounds. In specific cases, the composition comprises Rice Bean Extract and one or more other compounds. In specific cases, the composition comprises Wheat (Immature Fruit) Extract and one or more other compounds. In specific cases, the composition comprises Houttuynia (Aboveground Parts) Extract and one or more other compounds. In specific cases, the composition comprises Citron (Fruit) Extract and one or more other compounds. In specific cases, the composition comprises Sacred Lotus (Seed) Extract and one or more other compounds. In specific cases, the composition comprises Chinese Mint Extract and one or more other compounds. In specific cases, the composition comprises Butcher's Broom Extract and one or more other compounds. In specific cases, the composition comprises Oolong Tea (leaf) Extract and one or more other compounds. In specific cases, the composition comprises Senna Leaf Extract and one or more other compounds. In specific cases, the composition comprises Yohimbe bark Extract and one or more other compounds. In specific cases, the composition comprises Japanese Thistle extract and one or more other compounds. In specific cases, the composition comprises Butterbur Extract and one or more other compounds. In specific cases, the composition comprises Caralluma bark (wild) Extract and one or more other compounds. In specific cases, the composition comprises Caralluma bark (wild) Extract and one or more other compounds. In specific cases, the composition comprises Caralluma bark (wild) Extract and one or more other compounds. In specific cases, the composition comprises Echinacea root Extract and one or more other compounds. In specific cases, the composition comprises Horse Chestnut fruit Extract and one or more other compounds. In specific cases, the composition comprises Kola Nut Extract and one or more other compounds. In specific cases, the composition comprises Magnolia Bark Extract and one or more other compounds. In specific cases, the composition comprises s. Muira Puama Bark Extract and one or more other compound.

In particular embodiments, the composition comprises 1, 2, 3, 4, 5, 6, 7, 8, 9, 10, 11, 12, 13, 14, 15, 16, 17, or all of Euryale (Seed) Extract; Rice Bean Extract; Wheat (Immature Fruit) Extract; Houttuynia (Aboveground Parts) Extract; Citron (Fruit) Extract; Sacred Lotus (Seed) Extract; Chinese Mint Extract; Butcher's Broom Extract; Oolong Tea (leaf) Extract; Senna Leaf Extract; Yohimbe bark Extract; Japanese Thistle extract; Butterbur Extract; Caralluma bark (wild) Extract; Echinacea root Extract; Horse Chestnut fruit Extract; Kola Nut Extract; Magnolia Bark Extract; and Muira Puama Bark Extract.

In some embodiments, the composition comprises Lesser Galangal Extract (Alpinia officinarum), Honeysuckle (Flower Bud) Extract, Chinese Mint Extract, Clove (Flower Bud) Extract, Citron (Fruit) Extract or a combination thereof. In specific cases, the composition comprises Lesser Galangal Extract (Alpinia officinarum) and one or more other compounds. In specific cases, the composition comprises Honeysuckle (Flower Bud) and one or more other compounds. In specific cases, the composition comprises Chinese Mint Extract and one or more other compounds. In specific cases, the composition comprises Clove (Flower Bud) Extract and one or more other compounds. In specific cases, the composition comprises Citron (Fruit) Extract and one or more other compounds. In particular embodiments, the composition comprises 1, 2, 3, or all of Lesser Galangal Extract (Alpinia officinarum), Honeysuckle (Flower Bud) Extract; Chinese Mint Extract; Clove (Flower Bud) Extract; and Citron (Fruit) Extract.

In specific embodiments, the composition comprises Japanese thistle extract; Grape seeds extract; Amla extract; Pine bark extract; Chinese mint extract; or a combination thereof. In specific cases, the composition comprises Japanese thistle extract and one or more other compounds. In specific cases, the composition comprises Grape seeds extract and one or more other compounds. In specific cases, the composition comprises Amla extract and one or more other compounds. In specific cases, the composition comprises Pine bark extract and one or more other compounds. In specific cases, the composition comprises Chinese mint extract and one or more other compounds. In particular embodiments, the composition comprises 1, 2, 3, 4, or all of Japanese thistle extract; Grape seeds extract; Amla extract; Pine bark extract; and Chinese mint extract. In specific embodiments, the composition comprises at least Japanese thistle extract and Grape seeds extract.

In some embodiments, one or more compositions are included with folic acid and/or one or more derivatives thereof. One can utilize folic acid and its one or more of its three derivatives, dihydrofolic acid, tetrahydrofolic acid and 5-methyl tetrahydrofolate (5-MTHF) in employing the XO inhibition assay. IC₅₀ for folic acid and 5-MTHF are 0.75 uM and 35.6 uM, respectively. Dihydrofolic acid and tetrahydrofolic acid are more potent than folic acid, in at least some embodiments. Importantly, the XO inhibitory effect was studied of combinations of single herb medicine extract with folic acid or 5-MTHF. Nine out of 14 extracts tested showed an additive XO inhibitory effect with folic acid; and 10 out of 14 extracts tested showed an additive XO inhibitory effect with 5-MTHF.

One can characterize the additive or synergistic effect of different combinations of extracts and folic acid or 5-MTHF at different experimental conditions. One can select a few lead combination formulations of herb extracts with or without folic acid or 5-MTHF and determine those that have additive and/or synergistic effects on XO inhibitions, in some cases while dose of each component in the combination is relatively small. These lead combination recipes may be characterized in animal model tests and ultimately for the treatment and/prevention of patients with both symptomatic and asymptomatically hyperuricemia conditions, which are a significant and independent risk factor for cardiovascular disease and many other diseases. In addition, these herb extracts/folic acid-based health supplements also have antioxidant capability, reduce oxidative stress, and decrease serum homocysteine levels, thereby providing multiple health benefits.

### I. Pharmaceutical and Other Compositions

In accordance with this disclosure, the term “composition” or “pharmaceutical composition” relates to a composition for administration to an individual. In a particular embodiment, the pharmaceutical or other composition comprises a composition for parenteral, transdermal, intraluminal, intra-arterial, intrathecal or intravenous administration, for example. It is in particular envisaged that the pharmaceutical composition is administered to the individual orally. Administration of the suitable composition(s) may be effected by different ways, e.g., by oral, intravenous, subcutaneous, intraperitoneal, intramuscular, topical, intradermal administration, via infusion, injection, or rectal, including as using suppositories, for example.

The pharmaceutical or other composition of the present disclosure may further comprise an acceptable carrier, such as a pharmaceutically acceptable carrier. Examples of suitable carriers are well known in the art and include phosphate buffered saline solutions, water, emulsions, such as oil/water emulsions, various types of wetting agents, sterile solutions, etc. Compositions comprising such carriers can be formulated by well-known conventional methods. These compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A preferred dosage for administration might be in the range of 0.24 μg to 48 mg, preferably 0.24 μg to 24 mg, more preferably 0.24 μg to 2.4 mg, even more preferably 0.24 μg to 1.2 mg and most preferably 0.24 μg to 240 mg units per kilogram of body weight per day. Progress can be monitored by periodic assessment.

The compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a particular embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the composition.

In some embodiments, any of the compositions encompassed herein may be consumed or otherwise utilized by an individual once or more than once. When the composition is consumed or otherwise utilized more than once, the duration between administrations may be on the order of 1-24 hours, 1-7 days, 1-4 weeks, or 1-12 months or more. The individual may or may not be consuming or utilizing the compositions following a diagnosis indicating the individual was in need thereof. In particular aspects, the individual is diagnosed as having excessive uric acid levels, for example with respect to the general population or as a control, and the compositions are then consumed or utilized thereafter. The individual may obtain the compositions through commercial sources, including through the world wide web.

When there is more than one element (for example, type of extract) listed herein comprised within a composition as encompassed herein, the elements may or may not be in a specific ratio. For example, in some cases two different elements of the composition may be formulated within the composition to be in a 1:1 ratio, a 1:2 ratio, a 1:5 ratio, a 1:10 ratio, a 1:25 ratio, a 1:50 ratio, a 1:100 ratio, a 1:500 ratio, a 1:1000 ratio, a 1:10000 ratio and so forth. In cases wherein the composition comprises three elements (for example, types of extract), the ratio may be a 1:1:1 ratio, 1:2:1 ratio, 1:5:1 ratio, a 1:10:1 ratio, a 1:50:1 ratio, a 1:100:1 ratio, a 1:500:1 ratio, a 1:1000:1 ratio, a 1:1:2 ratio, a 1:1:5 ratio, a 1:1:10 ratio, a 1:1:50 ratio, a 1:1:100 ratio, a 1:1:500 ratio, a 1:1:1000 ratio, a 2:1:1 ratio, a 5:1:1 ratio, a 10:1:1 ratio, a 50:1:1 ratio, a 100:1:1 ratio, a 500:1:1 ratio, a 1000:1:1 ratio, and so forth.

In some embodiments, the concentration of one or more particular elements (for example, type of extract) may be within a range. For example, the one or more extracts may be within a range of concentration of 0.1 to 100 mg/kg in the composition. In specific embodiments, the one or more extracts may be within a range of concentration of 0.1 to 100 mg/kg; 0.1 to 75 mg/kg; 0.1 to 50 mg/kg; 0.1 to 25 mg/kg; 0.1 to 10 mg/kg/0.1 to 5 mg/kg; 0.1 to 1 mg/kg; 0.1 to 0.75 mg/kg/0.1 to 0.5 mg/kg; 0.5 to 100 mg/kg; 0.5 to 75 mg/kg; 0.5 to 50 mg/kg; 0.5 to 25 mg/kg; 0.5 to 10 mg/kg; 0.5 to 5 mg/kg; 0.5 to 1 mg/kg; 1 to 100 mg/kg; 1 to 75 mg/kg; 1 to 50 mg/kg; 1 to 25 mg/kg; 1 to 10 mg/kg; 1 to 5 mg/kg; 5 to 100 mg/kg; 5 to 75 mg/kg; 5 to 50 mg/kg; 5 to 25 mg/kg; 5 to 10 mg/kg; 10 to 100 mg/kg/10 to 75 mg/kg; 10 to 50 mg/kg/10 to 25 mg/kg; 25 to 100 mg/kg; 25 to 75 mg/kg; 25 to 50 mg/kg; 50 to 100 mg/kg; 50 to 75 mg/kg; or 75 to 100 mg/kg in the composition, as examples.

Any of the compositions described herein may be comprised in a kit. In a non-limiting example, one or more components of the composition(s) and/or the reagents to extract the herbs may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, the compositions are provided in a kit, and in some cases the compositions are essentially the sole component of the kit. The kit may comprise reagents and materials to make the desired extract or composition. In particular embodiments, there are one or more apparatuses in the kit suitable for extracting one or more samples from an individual. The apparatus may be a syringe, scalpel, and so forth.

In some cases, the kit, in addition to disclosed embodiments, also includes a second therapy, such as one or more uricosurics and/or one or more xanthine oxidase inhibitors other than those described in the disclosure, for example.

### II Dietary or Health Food Supplements

Any herbal components and non-herbal components of this disclosure can be used in the form of a dietary supplement or health food supplement or as a medicinal preparation, for example, in solid, semi-solid or liquid form that comprises the composition of the present disclosure, as an active ingredient, including in at least some cases in admixture with an organic or inorganic carrier or excipient suitable for external, enteral or parenteral applications. The active ingredient(s) may be compounded, for example, with the usual non-toxic pharmaceutically acceptable carriers for tablets, pellets, capsules, suppositories, solutions, emulsions, suspensions, and any other form suitable for use. Formulations of the present disclosure encompass those that include an exemplified carrier, including carriers such as water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid or liquid form and in addition auxiliary, stabilizing, thickening and coloring agents and perfumes may be used.

For preparing solid compositions such as tablets or capsules, the principal active ingredient(s) may be mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid preformulation composition comprising a substantially homogeneous mixture of one or more compositions of the present disclosure, or a non-toxic pharmaceutically acceptable salt thereof. In cases when referring to the preformulation compositions as substantially homogenous, it is meant that the active ingredients are dispersed evenly throughout the composition so that the composition may be readily subdivided into equally effective unit dosage forms such as tablets, pills and capsules. This solid preformulation composition may then be subdivided into unit dosage forms of the type described above containing 0.4 mg of the composition of the present disclosure, for example in capsules. The tablets or pills of the novel composition can be coated or otherwise compounded to provide a dosage form affording the advantage of prolonged action, in some cases. For example, the tablet or pill can comprise an inner dosage an outer dosage component, the latter being in the form of an envelope over the former. In some cases, the components can be separated by an enteric layer that serves to resist disintegration in the stomach and permits the inner component to pass intact into the duodenum or to be delayed in release. A variety of materials can be used for such enteric layers or coatings such materials, including a number of polymeric acids and mixtures of polymeric acids with such materials as shellac, cetyl alcohol and cellulose acetate.

In cases wherein liquid forms are utilized, in which the novel composition of the present disclosure may be incorporated for administration orally or by injection, for example, they may include an aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

In one embodiment, the composition is formulated as a powder to be mixed with a liquid, such as with a drink or broth.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For buccal administration, the composition may take the form of tablets or lozenges formulated in conventional manners.

The active compounds may be formulated for parenteral administration by injection, which includes using conventional catheterization techniques or infusion. Formulations for injection may be presented in unit dosage form, e.g., in ampules, or in multi-dose containers, with an added preservative. The composition(s) may take such forms as suspensions, solutions or emulsions in oily or aqueous vehicles, and may contain formulating agents such as suspending, stabilizing, and/or dispersing agents. Alternatively, the active ingredient(s) may be in powder form for reconstitution with a suitable vehicle, e.g., sterile pyrogen-free water, before use.

### III. Methods of Preventing or Treating a Medical Condition

In particular embodiments, a medical condition is prevented or treated using one or more compositions of the disclosure. The medical condition may be of any kind, and the medical condition may or may have not been diagnosed by a medical practitioner of any kind, including of Eastern or Western medicine. In specific embodiments, the medical condition is one that is directly or indirectly related to oxidative stress. The medical condition may be related to excessive levels of uric acid in the individual, such as compared to a standard or the general population. The compositions and methods of the disclosure may completely inhibit the medical condition, delay the onset of the medical condition or reduce the severity of one or more symptoms of the medical condition. The medical condition may be gout, hypertension, atherosclerosis, coronary artery disease, heart failure, left ventricular hypertrophy, atrial fibrillation, periphery artery disease, vascular restenosis, vascular thrombosis, stroke, diabetes, insulin resistance, metabolic syndrome, chronic kidney disease, psoriatic arthritis, micro-albuminuria, erectile dysfunction, preeclampsia, cancers, immune disorders, or an inflammatory disease.

In particular embodiments, the composition(s) may be provided to an individual in need thereof once or more than once. When provided more than once, the duration in time between administrations may be of any duration such as 1-60 minutes, −24, days, 1-4 weeks or 1-12 or more months. The dosage with multiple administrations may or may not be the same.

### EXAMPLES

The following examples are presented in order to more fully illustrate the preferred embodiments of the disclosure. They should in no way, however, be construed as limiting the broad scope of the disclosure.

### Example 1

### Examples of Materials and Methods

The inventors have carefully selected and ordered standard extracts from 72 traditional Chinese herb medicines (Table 1).

All 72 herb extracts (100 g) are in the powder form. Each extract was dissolved in water or DMSO respectively with strong votex, at stock concentration of 50 mg/ml. The solution was kept at room temperature for 30 min before centrifugation at 14k rpm. The clear solution was used for assay of XO inhibitory activity. XO activity was determined using the method of continuous spectrophotometric rate measurements (60). The reaction mixture contained xanthine in 67 mM phosphate buffer (pH 7.4) and 20 nM XO with an activity of 5 uU/mL, with or without extract solution. After pre-incubating the extract (at 0-10 ul) with XO for 1 min at 25° C., 50 uM xanthine was added to initiate the formation of uric acid, and the increase of absorption of uric acid at 295 nm was monitored in a dynamic mode. Initial rate of XO activity was recorded. 0-10 ul of DMSO was assayed and found that it has no inhibitory effect on XO activity. For initial screen of herb extracts, final concentration of each extract was 163 ug/ml. Percentage of inhibition is calculated: Percentage of inhibition=(1−test OD/blank OD)×100

Folic acid, dihydrofolic acid and tetrahydrofolic acid were purchased from Alfa Aesar (Tewksbury, M A); 5-methyl tetrahydrofolate (5-MTHF) was ordered from Sigma-Aldrich (St. Louis, Mo.); and 6-formylpterin was obtained from Cayman Chemical (Ann Arbor, Mich.). XO inhibition assay for folic acid related molecules is similar to that for herb extracts. Folic acids were dissolved in DMSO. Dose-dependent of folic acids and photolysis effect were studied. FDA-approved OX-inhibitor drug, Allopurinol, and small molecule XO inhibitor, DHNB, were included as a positive control.

### Example 2

### Development of Health Food Supplements/Antioxidants for Controlling Hyperuricemia and Oxidative Stress

Provided herein are xanthine oxidase (XO) inhibition assays for 72 standard extracts of Chinese traditional herb medicines (single dose experiment: 163 ug/ml with two factions). There are 14 herb extracts that showed a strong effect of OX inhibition (>40% either in water-soluble fraction or in DMSO-soluble fraction). There are 27 herb extracts, which have an XO inhibition rate from 20% to 40%. The rest of 31 herb extracts had a weak effect of XO inhibition (<20%). Per preliminary literature search, 7 out of 14 extracts (>40% XO inhibition) are not previously reported, and 12 out of 27 extracts (20-40% OXI) are not previously reported. DMSO-soluble fractions of these extracts are more potent XO inhibitors than their water-soluble fractions. The inventors performed a dose dependent study of XO inhibition for each of these 14 extracts (DMSO-soluble fractions). All 14 extracts showed a nice dose-dependent XO inhibition curve. IC₅₀ is an operational parameter defined as the concentration of inhibitor required for achieving 50% inhibition of the enzyme. The smaller IC₅₀, the more potent the inhibitor is. IC₅₀ for each extract is calculated. Top five potent XO inhibitors are Alpinia officinarum (IC₅₀ 28.5 ug/ml), Honeysuckle (IC₅₀ 33.5 ug/ml), Chinese Mint (IC₅₀ 34 ug/ml), Clove (Flower Bud)(IC₅₀ 63 ug/ml), and Citron fruit (IC₅₀ 71 ug/ml).

1. Data of 72 Standard Herb Medicine Extracts

Standard extracts of 72 traditional Chinese herb medicines in both water-soluble and DSMO-soluble fractions at a final concentration of 163 ug/ml were mixed with 20 nM XO, the initial rate of uric acid formation in the reaction system was recorded; and XO inhibition rate of each herb extract was calculated. All data are shown in Table 1. There are 14 herb extracts that showed a strong effect of XO inhibition (>40% either in water-soluble fraction or in DMSO-soluble fraction). There are 27 herb extracts, which have an XO inhibition rate between 20% to 40%. The rest of 31 herb extracts had a weak effect of XO inhibition (<20%).

72 items:

14 extracts: >40% XO inhibition (7/14 are not previously reported per our preliminary keyword search)

27 extracts: 20-40% XO inhibition (12 out of 27 are not previously reported per our preliminary keyword search)

31 extracts: <20% XO inhibition

TABLE 1
XO inhibitory effects of 72 traditional Chinese herb medicines
(standard extracts)

| | | DMSO % | H₂O, % | |
|---|---|---|---|---|
| | | of XO | of XO | Previous |
| Herb extract and Description | Code | inhibition¹ | inhibition² | report |
| #1, Extract 5:1 100 g \| Clove (Flower | EXD016 | 61 | 35 | Ref 61, 62, 63 |
| Bud) | | | | |
| #2, Extract 5:1 100 g \| Vietnamese | EXS007 | 71 | 22 | Ref 64, 65 |
| Sophora (Root) | | | | |
| Extract 5:1 100 g \| Fennel (Fruit) | EXX009 | 39 | 19 | NA |
| Extract 5:1 100 g \| Canadian Thistle | EXX010 | 30 | 35 | NA |
| (Aboveground Parts) | | | | |
| Extract 5:1 100 g \| Japanese Thistle | EXD003 | NA | 15 | |
| Extract 5:1 100 g \| Chinese Yam | EXS008 | 9 | 7 | |
| (Rhizome) | | | | |
| Extract 5:1 100 g \| Fructose Crataegi | EXS009 | 30 | 0 | Ref 66 |
| (Charred) | | | | |
| Extract 5:1 \| Fructus Crataegi | EXS010 | 5 | 0 | |
| Extract 5:1 100 g \| Smoked Plum/ | EXW007 | 5 | 4 | |
| Fructus Mume | | | | |
| Extract 5:1 100 g \| Flowering Quince | EXM009 | 52 | 28 | Ref 61, 67, 68 |
| (Fruit) | | | | |
| Extract 5:1 100 g \| Hemp Seed | EXH017 | 5 | 9 | |
| Extract 5:1 100 g \| Aromatic Solomon's | EXY014 | 4 | 9 | |
| Seal (Rhizomme) | | | | |
| Extract 5:1 100 g \| Chinese Licorice | EXG001 | 28 | 22 | Ref 66, 69 |
| (Root & Rhizome) | | | | |
| Extract 5:1 100 g \| Fragrant Angelica | EXB11 | 0 | | |
| Extract 5:1 100 g \| Ginkgo Leaf | EXY008 | 31 | 22 | Ref 70 |
| Extract 5:1 100 g \| White Hyacinth | EXB002 | 30 | 19 | NA |
| Bean | | | | |
| Extract 5:1 100 g \| Longan | EXG013 | 10 | 8 | |
| Extract 5:1 100 g \| Sickle-pod Senna | EXJ010 | 27 | 15 | Ref 71 |
| (Seed) | | | | |
| Extract 5:1 100 g \| Sea-ear Shell | EXJ023 | 30 | 22 | NA |
| Extract 5:1 \| Bulbus Lilii | EXB004 | 7 | 10 | |
| Extract 20:1 100 g \| Cortex Cinnamomi | EXR004 | 18 | 7 | |
| Extract 5:1 100 g \| Flesh-figer Citron | EXF004 | 0 | 0 | |
| Extract 5:1 100 g \| Bitter Apricot Seed | EXB019 | 0 | | |
| Extract 5:1 100 g \| Oyster Shell | EXM010 | 0 | | |
| #4, Extract 5:1 100 g \| Euryale (Seed) | EXQ002 | 42 | 30 | NA |
| #5, Extract 5:1 100 g \| Rice Bean | EXC010 | 44 | 27 | NA |
| Extract 5:1 100 g \| Fructus Hordei | EXC005 | 38 | 22 | NA |
| Germinatus | | | | |
| Extract 5:1 100 g \| Ophiopogon | EXM003 | 0 | | |
| #6, Extract 5:1 100 g \| Fringed Pink | EXQ009 | 45 | 18 | Ref 66 |
| (Aboveground Parts) | | | | |
| #7, Extract 10:1 100 g \| Wheat | EXF008 | 40 | 25 | NA |
| (Immature Fruit) | | | | |
| Extract 5:1 100 g \| Dried Dates | EXD006 | 0 | | |
| Extract 5:1 100 g \| Chinese Dwwarf | EXY012 | 12 | 0 | |
| Cherry Seed Extract 5:11 Semen Pruni | | | | |
| #8, Extract 5:1 100 g \| Honeysuckle | EXJ006 | 73 | 55 | Ref |
| (Flower Bud) | | | | 66, 72, 73, 74 |
| #9, Extract 5:1 100 g \| Houttuynia | EXY013 | 40 | 3 | NA |
| (Aboveground Parts) | | | | |
| Extract 10:1 100 g \| Ginger (Fresh | EXY018 | 5 | | |
| Rhizome) | | | | |
| Extract 10:1 100 g \| Rhizome Zingi | EXG002 | 0 | 0 | |
| Extract 5:1 | | | | |
| Extract 5:1 100 g\| Turmeric (Rhizome) | EXJ003 | 13 | 17 | |
| Extract 5:1 100 g \| Fructus Aurantii | EXZ009 | 23 | 17 | Ref 75 |
| Processed | | | | |
| [symbol] Extract 5:1 100 g \| Bitter Orange | EXZ012 | 10 | 20 | Ref 76 |
| (Young Fruit) | | | | |
| Extract 10:1 100 g \| Lycium (Fruit) | EXG007 | 0 | 0 | |
| Extract 5:1 100 g \| Gardenia (Fruit) | EXS011 | 0 | | |
| Extract 5:1 100 g \| Chinese Amomum | EXS005 | 10 | 20 | |
| (Fruit) | | | | |
| Extract 5:1 100 g \| Sterculia (Seed) | EXP001 | 22 | 4 | NA |
| Extract 5:1 100 g \| Poria (Sclerotium) | EXP005 | 10 | 10 | |
| Extract 5:1 100 g \| Chinese Smilax | EXT007 | 37 | 11 | Ref 66, 76, 77 |
| (Rhizome) | | | | |
| #10, Extract 5:1 100 g \| Citron (Fruit) | EXX008 | 50 | 21 | NA |
| Extract 5:1 100 g \| Haizhou Elsholtzia | EXZ005 | 38 | 26 | NA |
| (Aboveground Parts) | | | | |
| Extract 5:1 100 g \| Peach (Seed) | EXT002 | 10 | 0 | |
| Extract 5:1 100 g \| White Mulberry | EXS004 | 30 | 15 | Ref 66, 78 |
| (Young Twig) | | | | |
| Extract 5:1 100 g \|White Mulberry | EXS003 | 22 | 7 | Ref 66, 79 |
| (Leaf) | | | | |
| Extract 5:1 100 g \| White Mulberry | EXS002 | 0 | | |
| (Fruit) | | | | |
| Extract 5:1 100 g \| Platycodon | EXJ004 | 0 | | |
| grandiflorus (Jacq.)A. DC. | | | | |
| Extract 5:1 100 g \| Sharp-Leaf Galangal | EXY006 | 32 | 18 | NA |
| (Seed) | | | | |
| Extract 5:1 100 g \| Sacred Lotus (Leaf) | EXH005 | 31 | 17 | Ref 80 |
| Extract 5:1 100 g \| Radish (Seed) | EXL001 | 20 | 20 | NA |
| #11, Extract 5:1 100 g \| Sacred Lotus | EXL005 | 46 | 16 | NA |
| (Seed) | | | | |
| Extract 5:1 100 g \| Sacred Lotus | EXL006 | 29 | 8 | NA |
| (Plumule) | | | | |
| #12, Extract 5:1 100 g \| Lesser Galangal | EXG004 | 65 | 23 | Ref 81.82.83 |
| (also called Alpinia officinarum) | | | | |
| Extract 5:1 100 g \| Lophatherum | EXD009 | 10 | 0 | |
| Extract 5:1 100 g \| Fermented Soybean | EXD007 | 24 | 13 | NA |
| #13, Extract 5:1 100 g \| Wild | EXY003 | 48 | 34 | Ref |
| Chrysanthemum (Flower) | | | | 66, 84, 85, 86.87 |
| Extract 5:1 100 g \| Chrysanthemum | EXB008 | 27 | 16 | Ref |
| (Flower) | | | | 66, 84, 85, 86, 87 |
| Extract 5:1 100 g \| Polygonatum | EXZ008 | 0 | | |
| (Rhizome, Cured) | | | | |
| Extract 5:1 100 g \| Perilla (Leaf) | EXZ018 | 32 | 17 | Ref 88, 89 |
| Extract 5:1 100 g \|Kudzu | EXG005 | 0 | | |
| Extract 5:1 100 g \| Herba Taraxaci | EXP004 | 15 | 15 | |
| Extract 5:1 100 g \| Jujube (Seed) | EXS027 | 32 | 21 | NA |
| Extract 5:1 100 g \| Phragmites | EXL010 | 17 | 10 | |
| (Rhizome) | | | | |
| #14, Extract 5:1 100 g \| Chinese Mint | EXB020 | 80 | 71 | NA |
| Extract 5:1 100 g \| Job's Tears | EXYOO5 | 37 | 15 | Ref 66, 90 |
| Extract 5:1 100 g \| Palm-leaf Raspberry | EXF006 | 10 | 30 | Ref 91 |
| Extract 5:1 100 g \| Patchouli | EXH018 | 35 | 20 | Ref 92 |
| (Aboveground Parts) | | | | |

NA means: the extract was not previously reported for XO inhibition
Blank means: the extract has no XO inhibition as tested, and no literature search was performed
¹DMSO fraction for the extract
²Water fraction for the extract

In general, most DMSO-soluble fractions of many herb extracts have a stronger effect of XO inhibition than their water-soluble fractions. These data indicates that potent XO inhibitory compounds are hydrophobic, in specific embodiments.

2. Data of Dose-Dependent of 14 Standard Herb Medicine Extracts

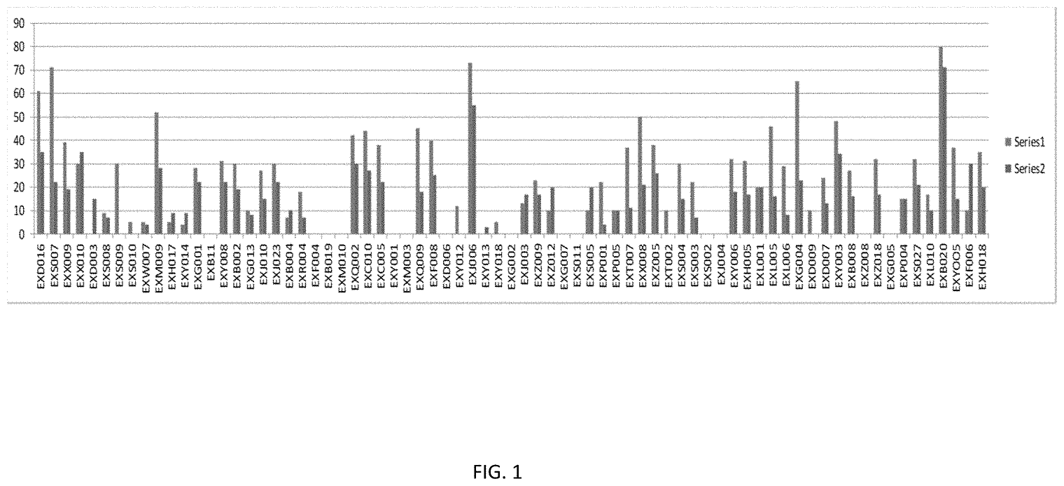

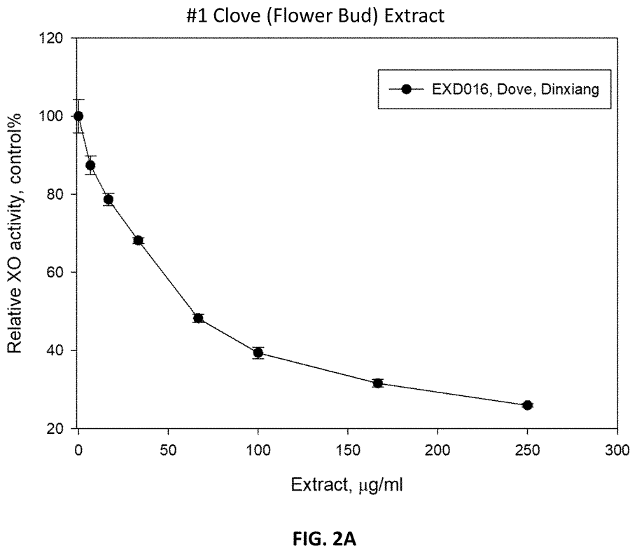

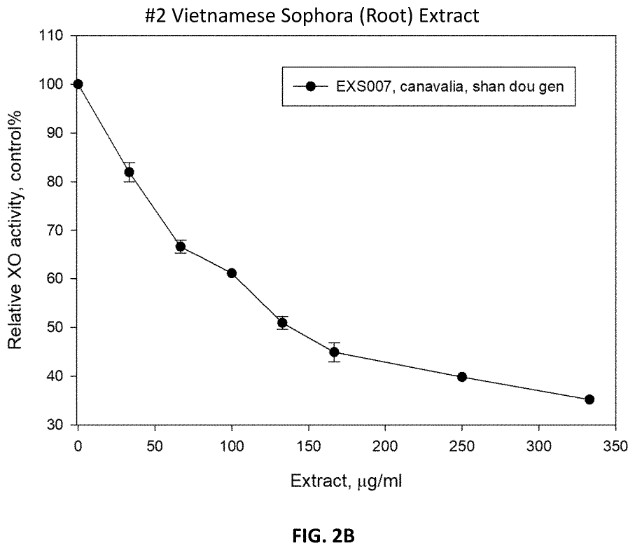

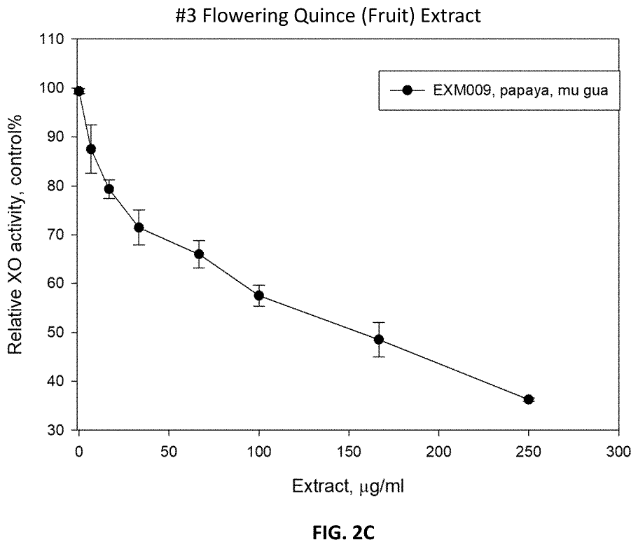

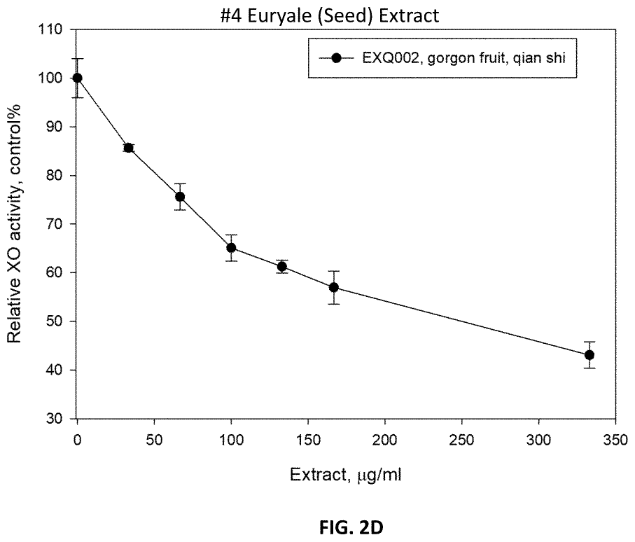

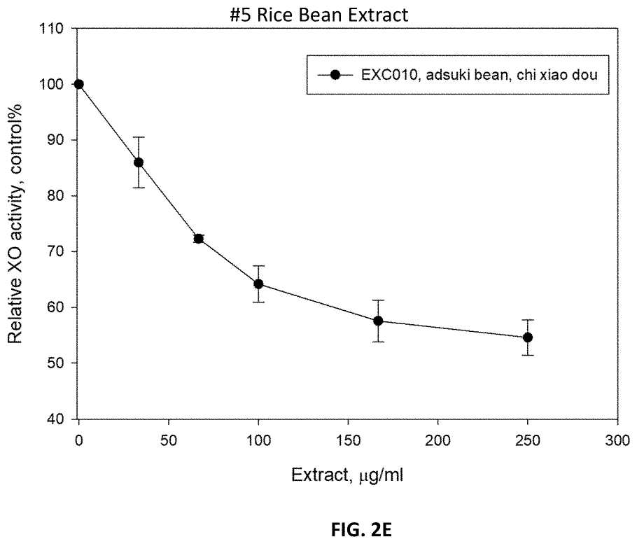

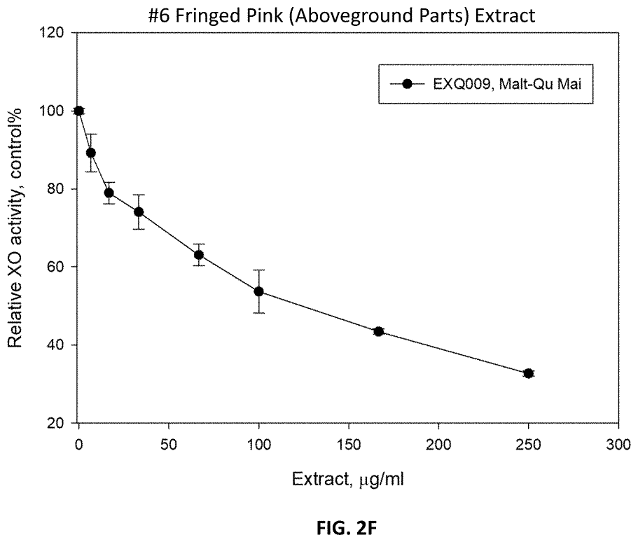

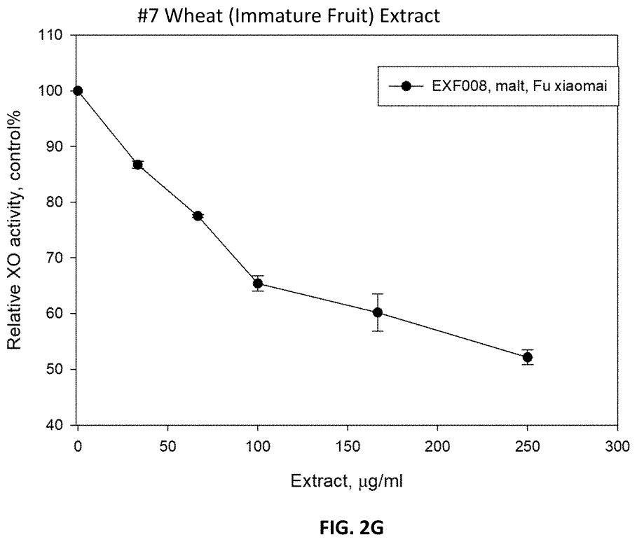

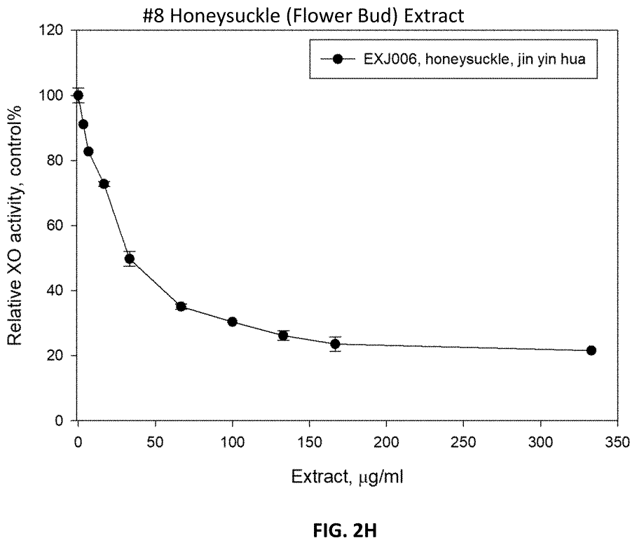

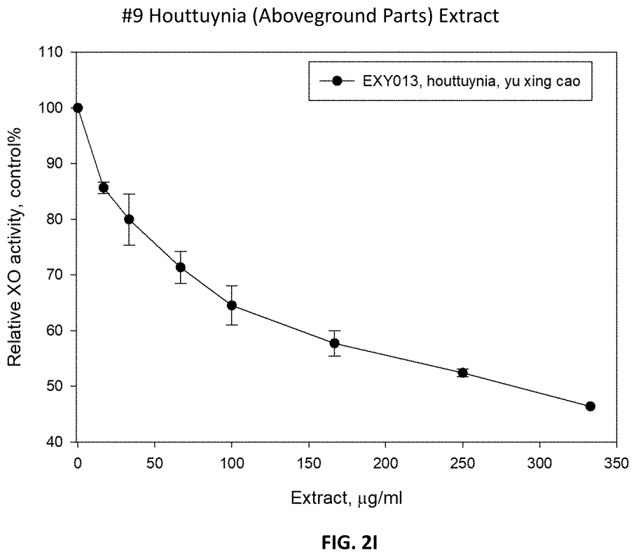

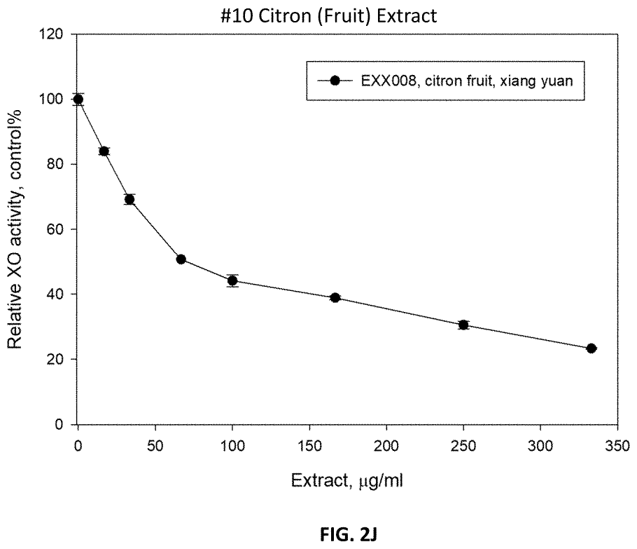

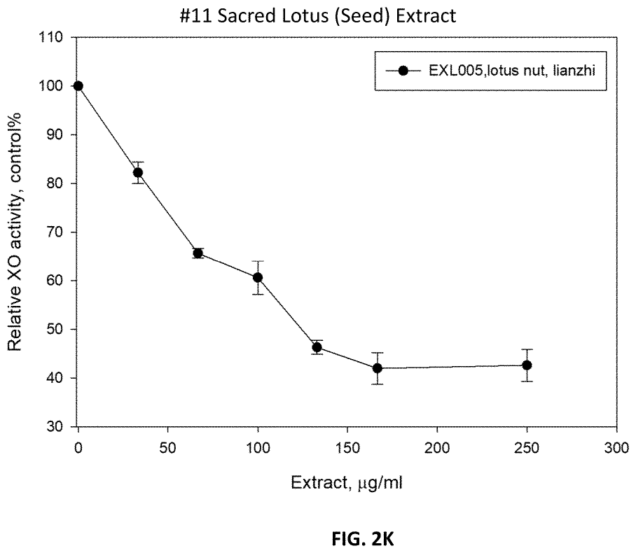

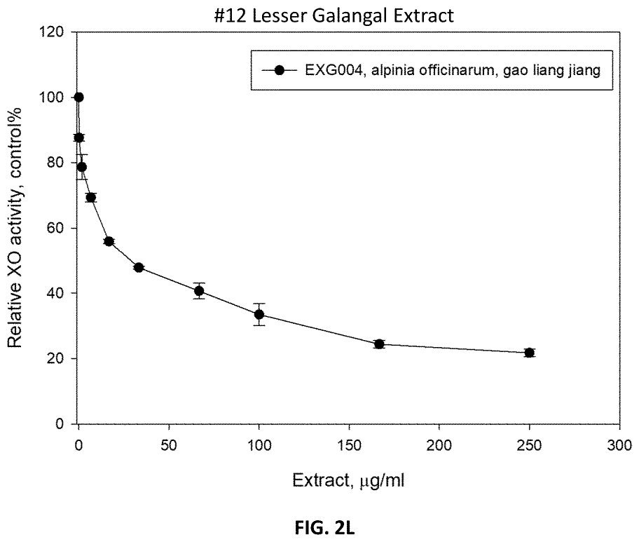

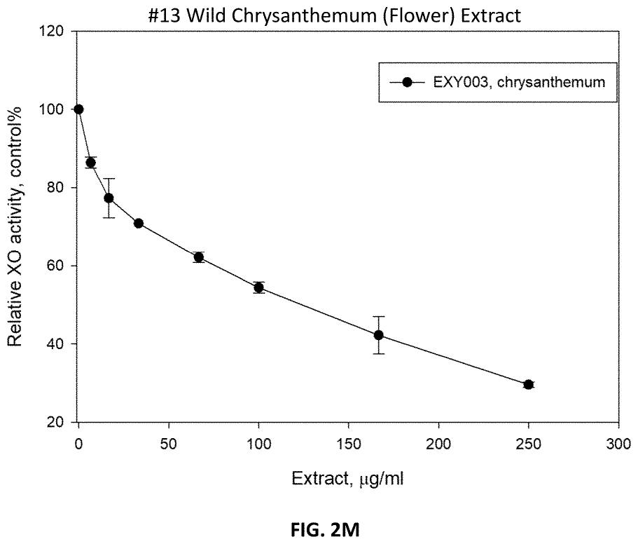

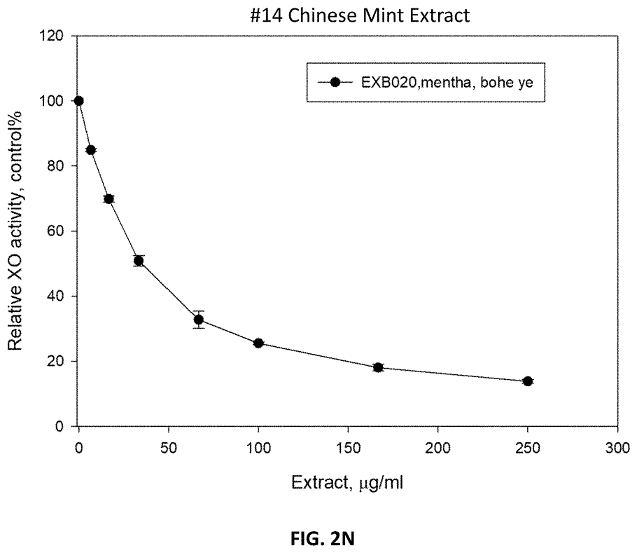

From initial screen of XO inhibitory effects of 72 traditional Chinese herb standard extracts with single dose (163 ug/ml), 14 extracts had more than 40% inhibition of XO activity. DMSO-soluble fractions of these extracts are more potent XO inhibitors than their water-soluble fractions (FIG. 1). Furthermore, we performed a dose dependent XO inhibition assay for each of these 14 extracts (DMSO-soluble fractions). Concentration range of each extract from 0 to 250, or 333 ug/ml (8 to 10 doses was studied). All 14 extracts showed a nice dose-dependent XO inhibition curve (FIG. 2). IC₅₀ is an operational parameter defined as the concentration of inhibitor required for achieving 50% inhibition of the enzyme. The smaller IC₅₀, the more potent the inhibitor is. IC₅₀ for each extract is calculated (Table 2). Top five potent XO inhibitors are Alpinia officinarum (IC₅₀ 28.5 ug/ml), Honeysuckle (IC₅₀ 33.5 ug/ml), Chinese Mint (IC₅₀ 34 ug/ml), Clove (IC₅₀ 63 ug/ml), and Citron fruit (IC₅₀ 71 ug/ml). These data are useful for understanding the potency of these extracts and provides rational design for combination assay of each extract with folic acid and 5-MTHF (for example) or multiple extracts with folic acid and 5-MTHF (for example).

TABLE 2
IC₅₀ (XO inhibitory effect) of 14 traditional Chinese herb medicines
(standard extracts)

| | | DMSO | | | |
|---|---|---|---|---|---|
| | | % | H₂O, % | | |
| | | of XO | Of XO | IC50 | Previous |
| Herb extract and Description | Code | inhibition | inhibition | (ug/ml) | report |
| #1, Extract 5:1 100 g \| Clove | EXD016 | 61 | 35 | 63 | Ref 61, 62, 63 |
| (Flower Bud) | | | | | |
| #2, Extract 5:1 100 g \| | EXS007 | 71 | 22 | 136 | Ref 64, 65 |
| Vietnamese Sophora (Root) | | | | | |
| #3, Extract 5:1 100 g \| | EXM009 | 52 | 28 | 154 | Ref 61, 67, 68 |
| Flowering Quince (Fruit) | | | | | |
| #4, Extract 5:1 100 g \| Euryale | EXQ002 | 42 | 30 | 246 | NA |
| (Seed) | | | | | |
| #5, Extract 5:1 100 g \| Rice | EXC010 | 44 | 27 | >300 | NA |
| Bean | | | | | |
| #6, Extract 5:1 100 g \| Fringed | EXQ009 | 45 | 18 | 120 | Ref 66 |
| Pink (Aboveground Parts) | | | | | |
| #7, Extract 10:1 100 g \| Wheat | EXF008 | 40 | 25 | 270 | NA |
| (Immature Fruit) | | | | | |
| #8, Extract 5:1 100 g \| | EXJ006 | 73 | 55 | 33.5 | Ref |
| Honeysuckle (Flower Bud) | | | | | 66, 72, 73, 74 |
| #9, Extract 5:1 100 g \| | EXY013 | 40 | 3 | 285 | NA |
| Houttuynia (Aboveground | | | | | |
| Parts) | | | | | |
| #10, Extract 5:1 100 g \| Citron | EXX008 | 50 | 21 | 71 | NA |
| (Fruit) | | | | | |
| #11, Extract 5:1 100 g \| Sacred | EXL005 | 46 | 16 | 123 | NA |
| Lotus (Seed) | | | | | |
| #12, Extract 5:1 100 g \| Lesser | EXG004 | 65 | 23 | 28.5 | Ref 81.82.83 |
| Galangal | | | | | |
| #13, Extract 5:1 100 g \| Wild | EXY003 | 48 | 34 | 123 | Ref |
| Chrysanthemum (Flower) | | | | | 66, 84, 85, 86.87 |
| #14, Extract 5:1 100 g \| | EXB020 | 80 | 71 | 34 | NA |
| Chinese Mint | | | | | |

NA means: the extract was not previously reported for XO inhibition.
Blank means: the extract has no XO inhibition as tested, and no literature search was performed.

Data of Folic Acid and its Derivatives

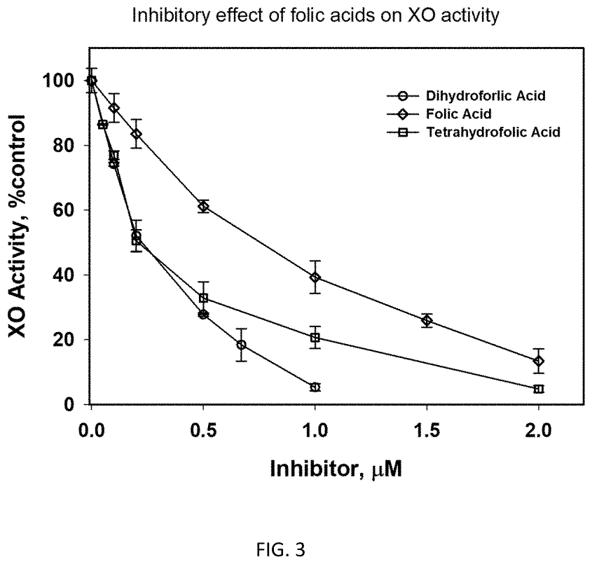

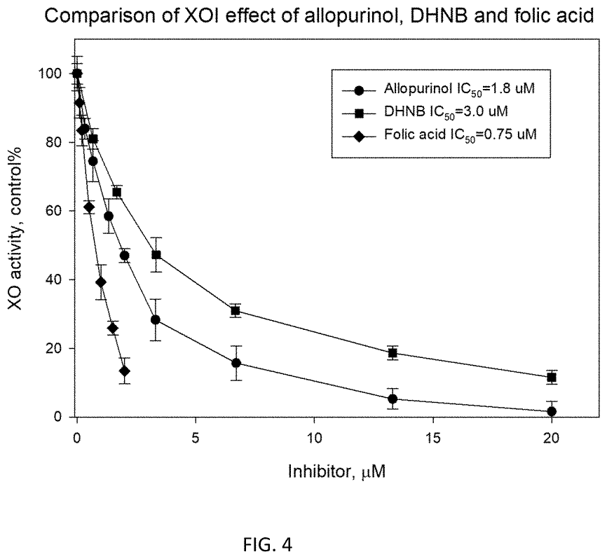

Folic acid and its two derivatives, dihydrofolic acid and tetrahydrofolic acid, were employed in the XO inhibition assay. When 20 nM XO was mixed with increasing concentrations of folic acid, dihydrofolic acid and tetrahydrofolic acid. The initial rate of uric acid formation showed a concentration-dependent decrease compared to the negative control, reflecting the decrease of XO activity (FIG. 3). Folic acid, dihydrofolic acid and tetrahydrofolic acid significantly inhibited XO activity with a dose range within 2 uM. Potency of XO inhibition shows dihydrofolic acid>tetrahydrofolic>folic acid. As compared to Allopurinol and DHNB, folic acid had an XO inhibitory effect stronger than Allopurinol and DHNB (FIG. 4). The half maximal inhibitory concentration (IC₅₀) for folic acid, allopurinol and DNHB is 0.75 uM, 1.8 uM and 3.0 uM, respectively.

Significance of Certain Embodiments (Folic Acid is a XO Inhibitor)

Folate and folic acid are forms of a water-soluble B vitamin. It is essential for the body to make DNA, RNA, and metabolize amino acids, which are required for cell division and many physiological functions. As humans cannot make folic acid, it is required from the diet, making it an essential vitamin. Folate is the common form of vitamin B9 present in many whole foods, including leafy greens, beans, eggs, citrus fruit, avocados, and beef liver. While folic acid is a synthesized version of vitamin B9 that is added to processed foods and the common version used in supplements. Folic acid is an oxidized form of folate, and has a molecular structure that is nearly identical to folate (a salt form). Folic acid is more stable than physiological types of folate. Since 1998, folic acid has been added to cold cereals, flour, breads, pasta, bakery items, cookies, and crackers, as required by US federal law. More than 50 countries are doing this. The goal of this regulation is an attempt to reduce the prevalence of Neural Tube Defects (NTDs), a common birth defect that had shown some connection to the mother's vitamin B9 intake. Besides NTDs, folate deficiency can lead to many health problems such as slowed growth, megaloblastic anemia, weight loss, digestive disorders, leukopenia, thrombocytopenia, cracking/redness of tongue/mouth, diarrhea, liver disease, cancers, cardiovascular disease, depression and other behavior changes (93-96). Thus, folic acid supplement has enormous impact on the improvement and maintenance of human health including prevention and treatment of many diseases.

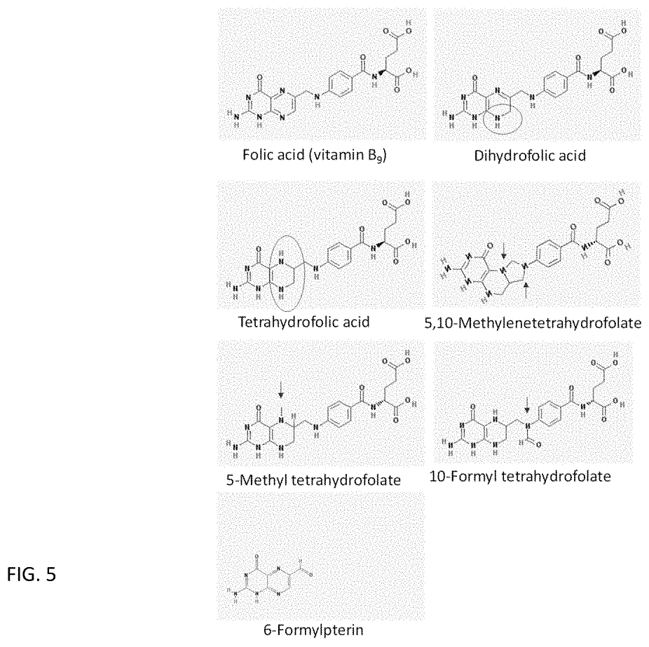

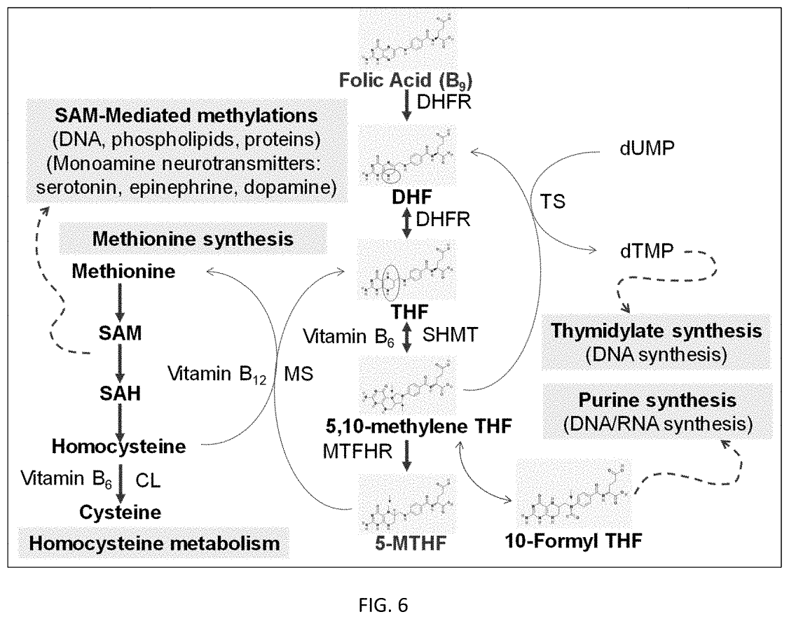

Before folic acid can be utilized by the body, it must undergo two conversions to dihydrofolate (DHF) first and then tetrahydrofolate (THF). THF can be converted to 5,10-methylenetetrahydrofolate (5,10-MTHFR) and 10-formyl tetrahydrofolate (10-FTHF), and 5-methyl tetrahydrofolate (5-MTHF, levomefolic acid), which is the end form of folate that the body requires for many key functions (FIG. 5). One of the most important folate-dependent reactions is the methylation of deoxyuridylate to thymidylate in the formation of DNA, is required for proper cell division. An impairment of this reaction initiates a process that can lead to megaloblastic anemia, one of the hallmarks of folate deficiency. Another folate-dependent reaction is the conversion of homocysteine to methionine in the synthesis of S-adenosyl-methionine. Hyperhomocyteinemia (Hcy) is toxic, well known risk factor for cardiovascular diseases and many other diseases (97). The chemical reactions that are necessary for degradation of Hcy require the presence of the folic acid, vitamins B6 and B12. Fold acid is also important for methylation of DNA, phospholipids and proteins for their functional regulations as well as synthesis of several important neurotransmitters such as serotonin, epinephrine and dopamine (FIG. 6). Furthermore, there is evidence that folic acid exerts both direct and indirect antioxidant effects, such as free radical scavenging (98).

Methylenetetrahydrofolate reductase (MTHFR) is the key enzyme that regulates this conversion process. However, it is estimated that up to 60% of Americans have genetic variations that reduce their ability to convert 5-MTHF from folic acid on their own. For those who have this genetic variation, supplementing with folic acid may lead to varying amounts of the converted 5-MTHF form, potentially leaving the body with less than it requires. Furthermore, it could lead to a build-up of the folic acid form in the body. This is why supplementing with 5-MTHF can be considered superior to using folic acid for those that may not get enough from the diet. THF is active only in its (unstable) reduced form and thus not suitable for oral supplementation. In general, supplements of folic acid and 5-MTHF at a recommended amount is safe and beneficial. The risk of toxicity from folic acid is low because folate is a water-soluble vitamin and is regularly removed from the body through urine (99). Folic acid intake below the established tolerable upper intake level (UL) of 1000 μg/day for the general population is not associated with any adverse health outcomes (100). However, excessive folic acid intake may be correlated with significant health risks including cancer (101-103); this risk issue is still under debate, not confirmed yet. In fact, folate deficiency increases the initiation of carcinogenesis by causing genome instability as well as alteration of methylation patterns in the genome, leading to altered expression of oncogenes and tumor suppressor genes (100).

The effect of folic acid on XO inhibition was first reported in 1948 (104); subsequently, the same research team reported that this inhibition was due to pterine-6-aldehyde (2-NH,-4-OH-pteridine-6-aldehyde), a photolytic breakdown product of folic acid in 1950 (105). This argument continued in 1980s (106, 107). Up to 1989, further experiments with different methods confirm that uric acid and its derivatives indeed are potent XO inhibitors (108). XO inhibitory effects of THF and DHF are stronger than that of folic acid (107, 109). Studies have shown that 5-MTHF can effectively inhibit XO activity in vitro and in vivo (110). Folic acid is known to be sensitive to ultraviolet (UV) radiation (111-116); it can be cleaved into p-aminobenzoyl-L-glutamic acid and 6-formyl pterin when exposed to ultraviolet radiation. When the irradiation continues, 6-formyl pterin is degraded to pterin-6-carboxylic acid (117). 6-formyl pterin is a very potent XO inhibitor in vitro; while pterin-6-carboxylic acid has no XO inhibitory effect (117).

Like humans, birds such as chicken and quail lack the enzyme uricase; their blood uric acid levels are usually higher than that in mice or rats that have the enzyme uricase, metabolizing uric acid to 5-hydroxyisourate (118). In fact, gout is a common disease in chicken, and their blood levels of uric acid can be as high as 44 mg/dL as compared to 5-7 mg/dL in a normal bird. Dietary folic acid supplementation significantly inhibits liver XO activities in the chick (119,120). A recent study showed that dietary folic acid supplementation significantly reduced serum uric acid in the older laying hens (121). Intraperitoneal administration of folic acid and allopurinol (12.5-50 mg/kg) significantly inhibited radiation-induced activation of XO in a mouse model (122). Daily oral supplement of folic acid significantly reduced serum uric acid levels and its associated renal pathology in a rat model (123). In addition, folic acid supplementation can prevent cardiovascular disease (124-126) and renal disease (127), inhibit tumorigenesis (128) and enhance the efficacy of antitumor chemotherapy (129) in animal models.

Studies of dietary supplement of folic acid for patients with hyperuricemia and/or gout have been reported. A case-control study showed that high folate intake may protect against gout (130). In adult hypertensive patients, the administration of a daily dose of 10 mg of Enalapril combined with 0.8 mg of folic acid had a greater beneficial effect on the serum UA levels than did that of 10 mg of Enalapril alone in patients with either an elevated UA concentration or baseline hyperuricemia (54,55). Furthermore, folic acid supplementation can reduce risks for cardiovascular disease and chronic kidney disease in clinical trials (56-59, 131).

One can confirm the efficacy of folic acid or 5-MTHF on control of hyperuricemia and in specific embodiments include it into one or more combination recipes with traditional Chinese herb medicines or other botanical ingredients as of dietary health supplements for long term management of hyperuricemia, as one example. Folic acid may be one of the components of any composition encompassed herein, in certain embodiments, and in specific embodiments it enhances the therapeutic efficacy of the combination therapy.

4. Data of Combination of Single Herb Medicine Extract with Folic Acid

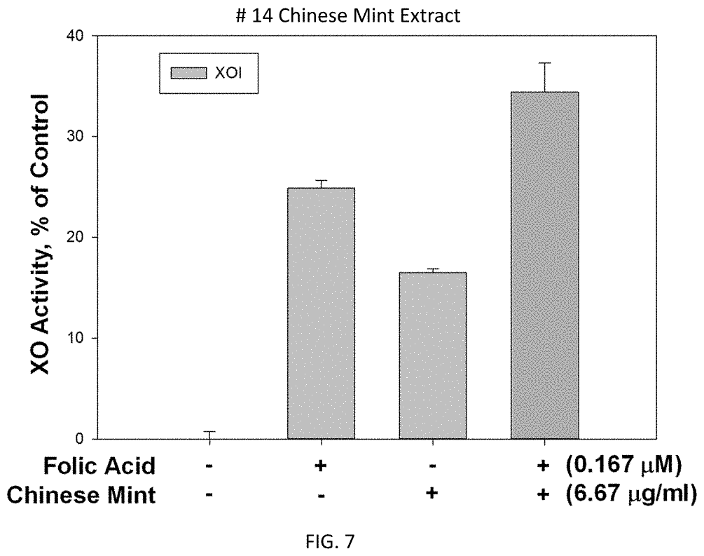

Based on the result of dose-dependent XO inhibition experiments for herb extracts and folic acid, a relatively small concentration of each extract (a total of 14 extracts) and folic acid were utilized for a combination assay. Initially, Chinese mint leave extract (6.67 ug/ml) and folic acid (0.167 uM) inhibited XO activity by 25.85% and 16.49%, respectively. The combination of Chinese mint leave extract and folic acid at these concentrations showed XO inhibition by 34.4% (FIG. 7). It is clear that the combination of Chinese mint leave extract and folic acid shows an additive effect on XO inhibition. One of the important advantages of such combination supplements is enhancement of XO inhibitory function of the supplement; while reducing the dose of each component, thereby reducing potential side effects of the supplement for long-term use.

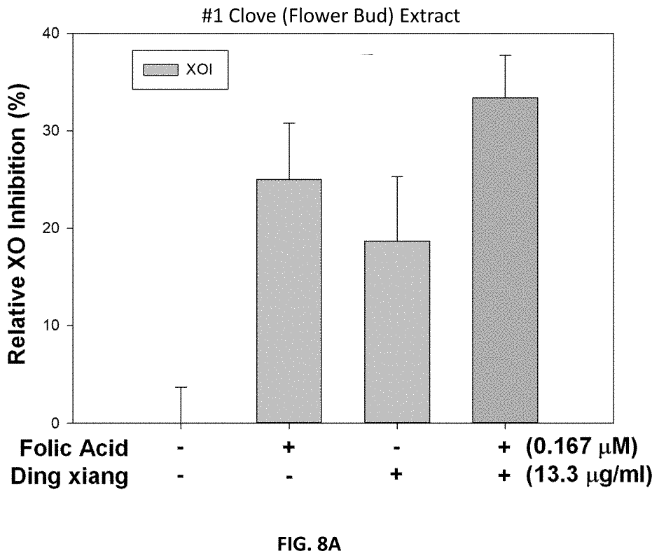

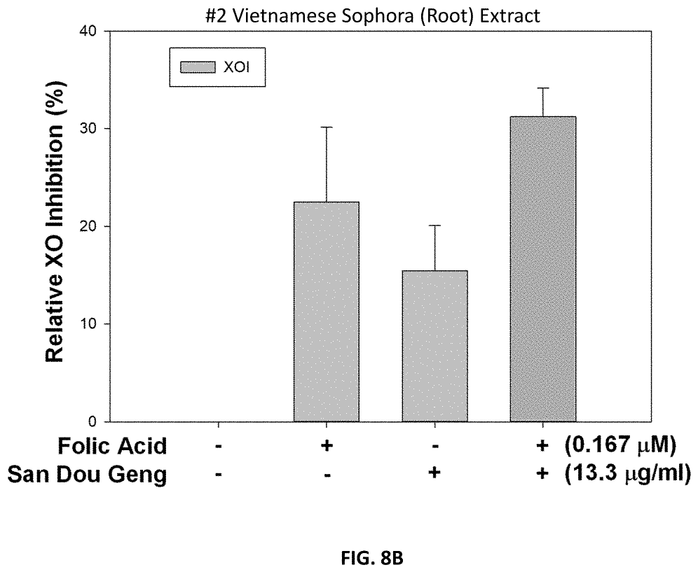

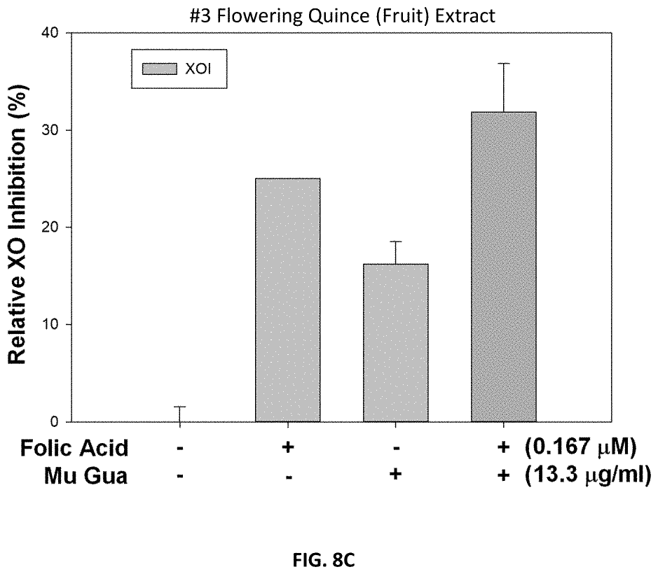

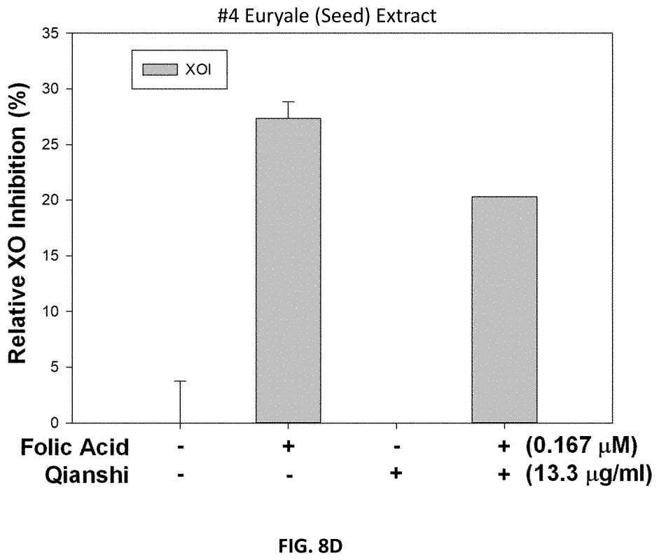

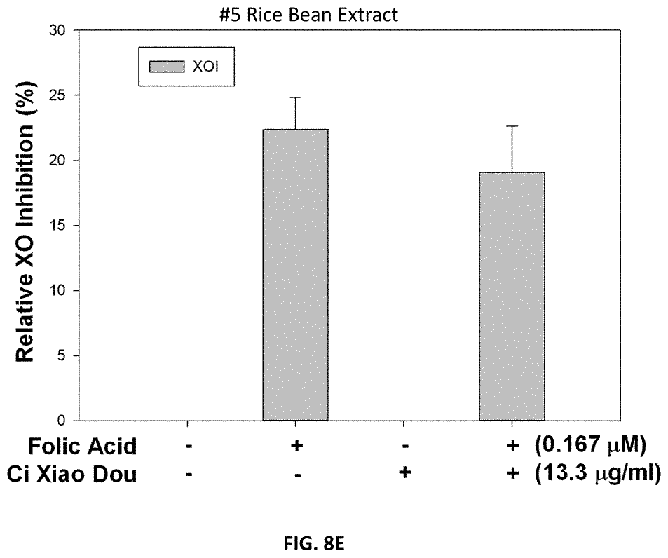

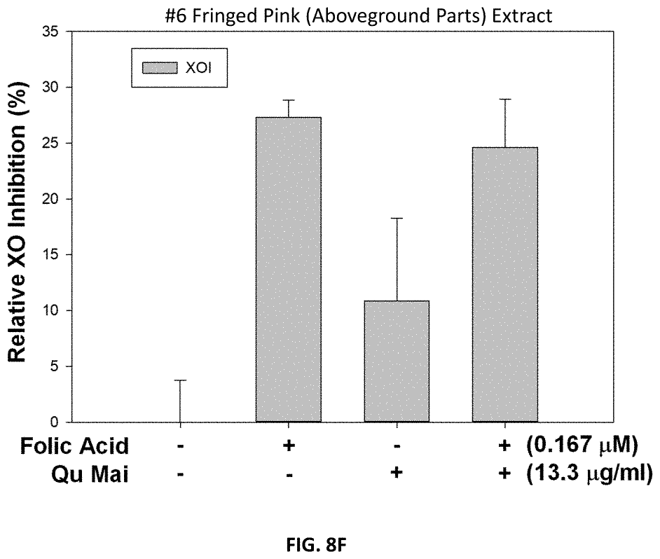

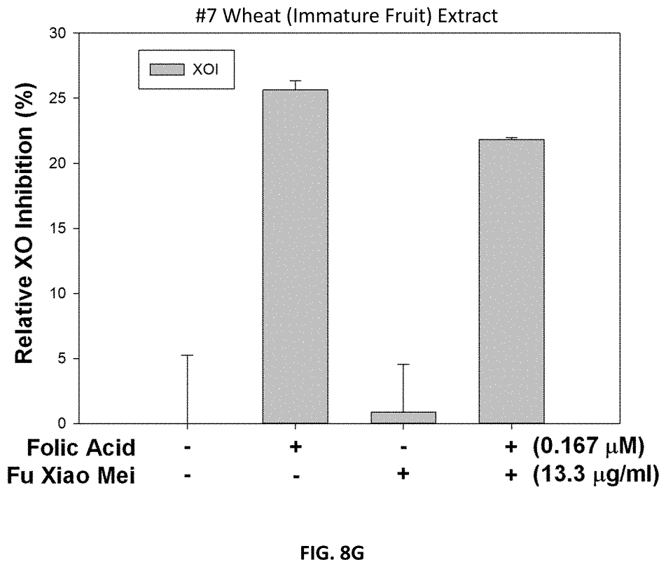

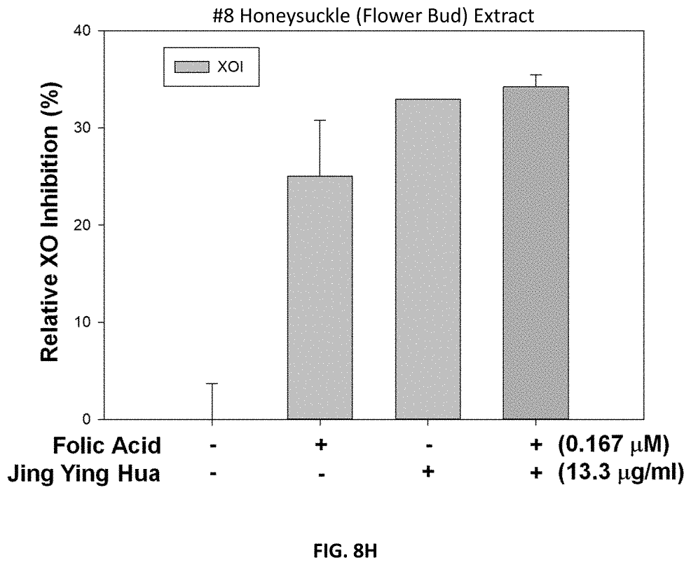

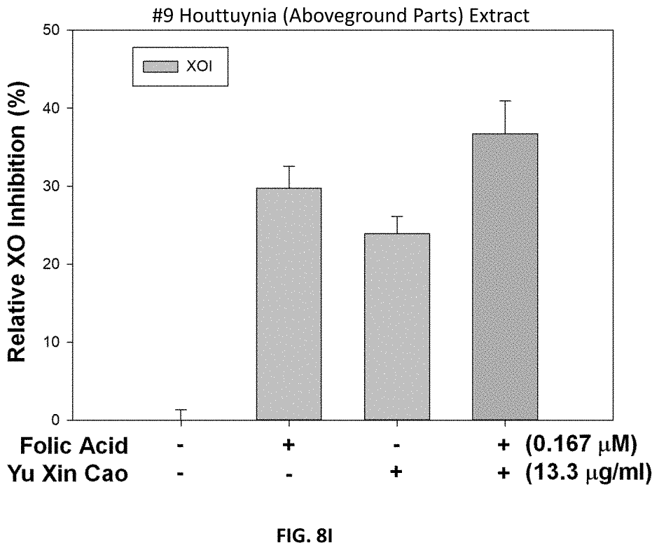

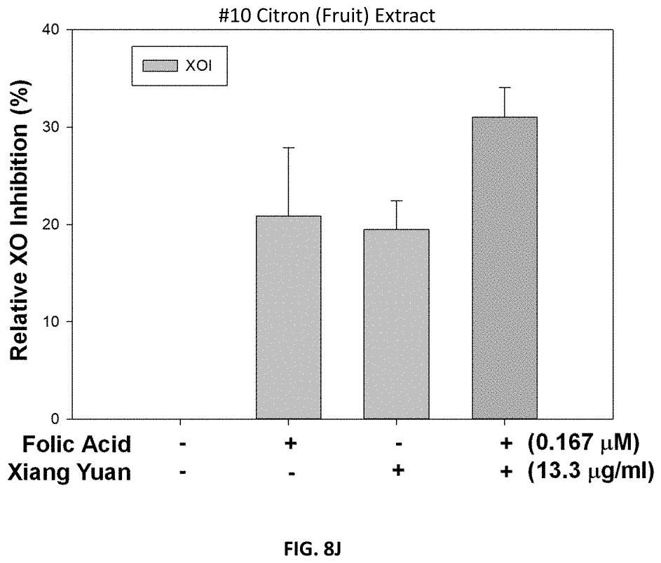

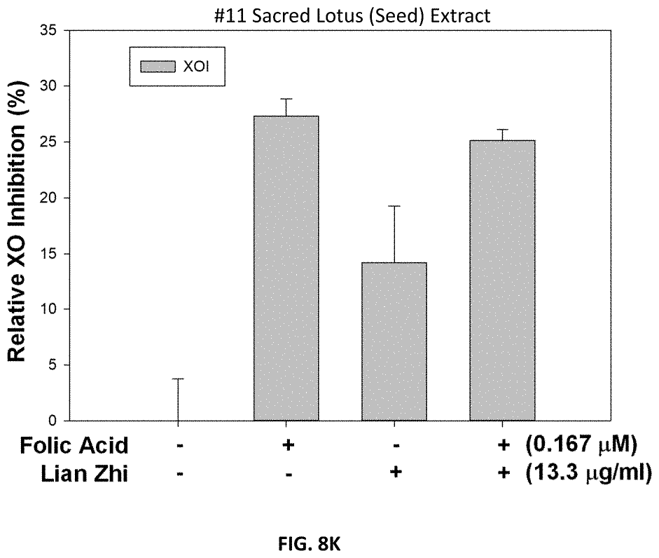

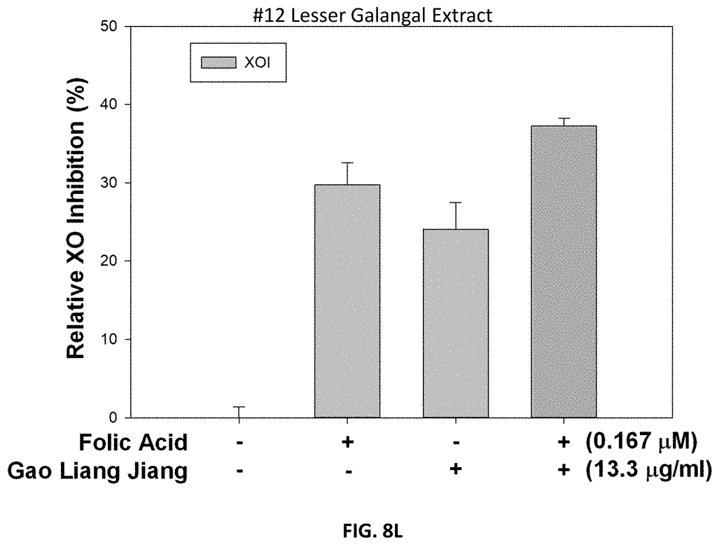

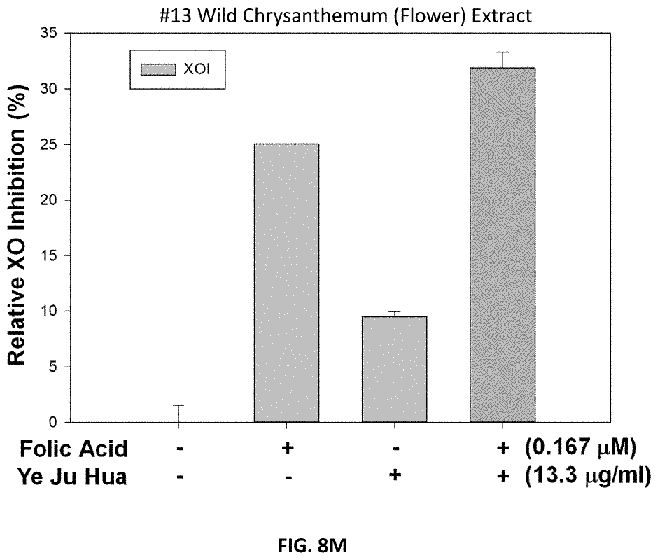

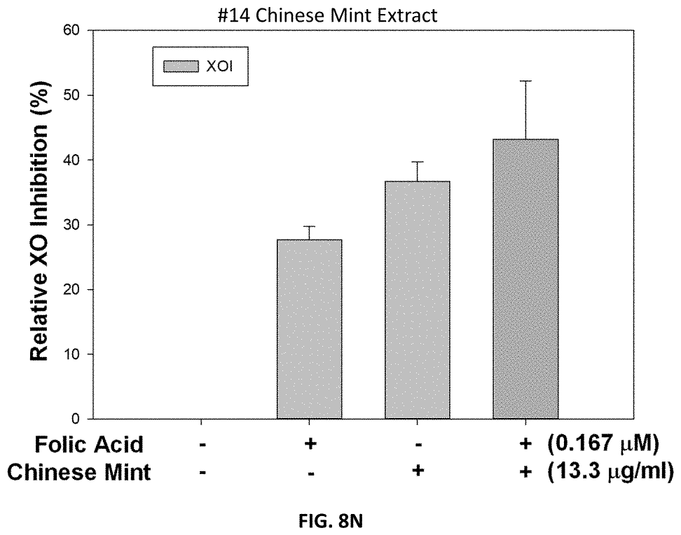

Furthermore, a combination essay was performed for folic acid and each of all 14 traditional Chinese medicine herb extracts including Chinese mint extract. Folic acid (0.167 uM) and extract (13.3 ug/ml) were separately added into the XO reaction system without pre-mixing. XO activity was measured. 9 out of 14 extracts showed an additive XO inhibitory effect with folic acid (FIG. 8). These effective extracts are Chinese mint extract; |Clove (Flower Bud) Extract; Vietnamese Sophora (Root) Extract; Flowering Quince (Fruit) Extract; Honeysuckle (Flower Bud) Extract; Houttuynia (Aboveground Parts) Extract; Citron (Fruit) Extract; Lesser Galangal Extract; and Wild Chrysanthemum (Flower) Extract.

5. Data of Combination of Herb Medicine Extracts with 5-Methyl Tetrahydrofolate (5-MTHF)

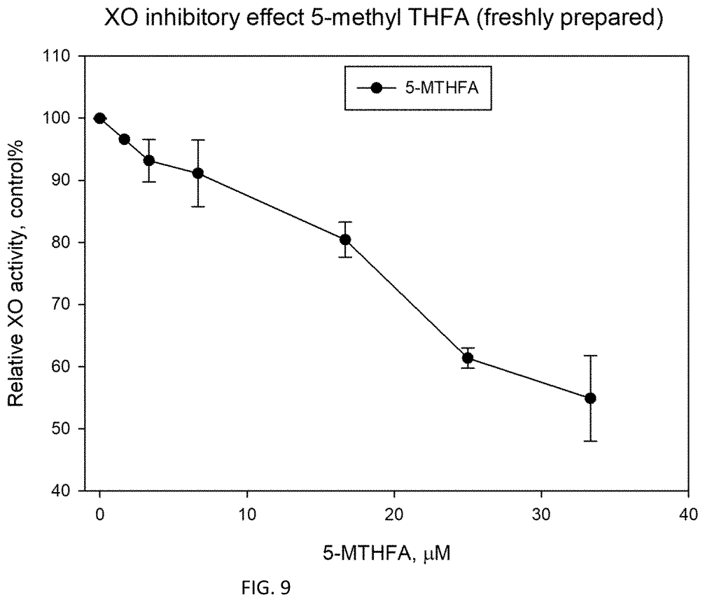

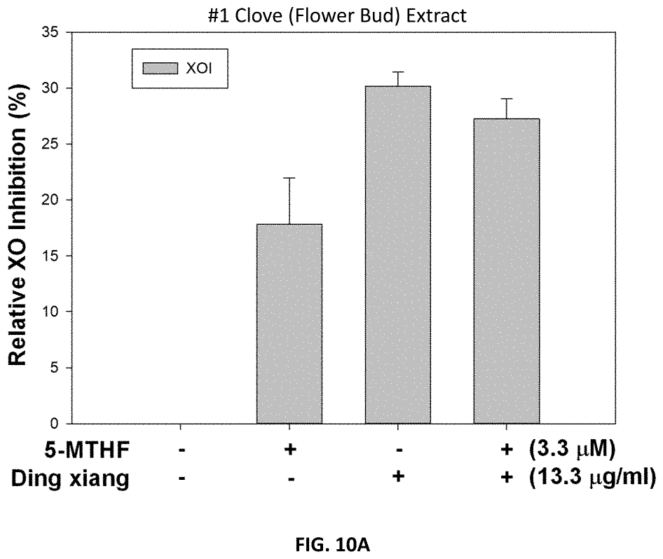

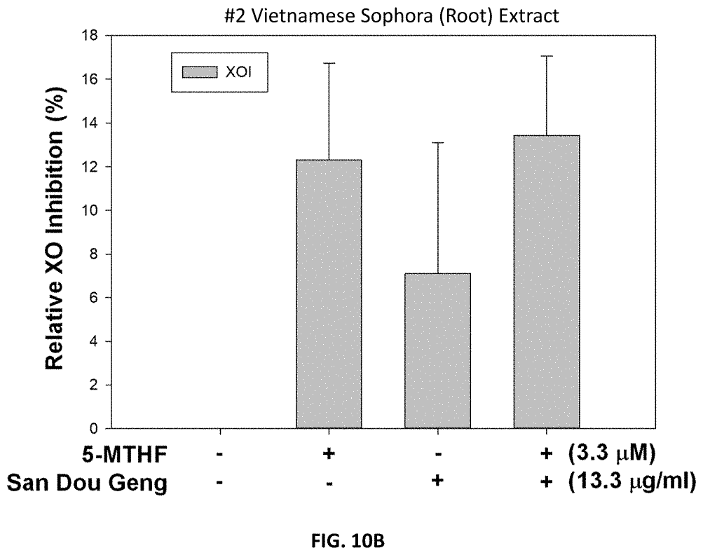

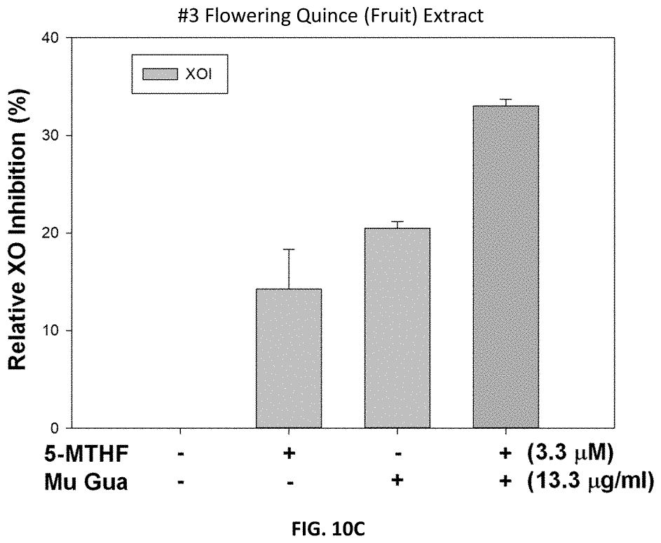

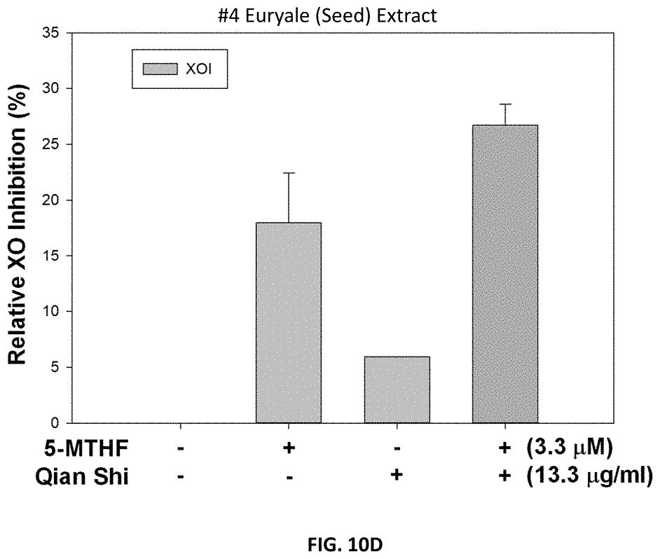

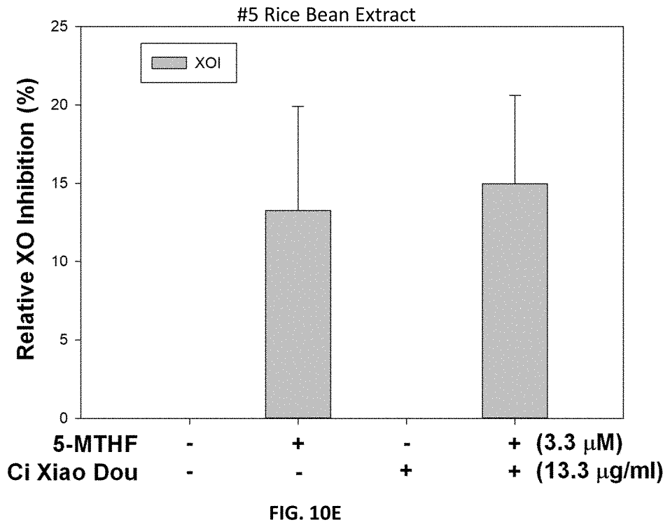

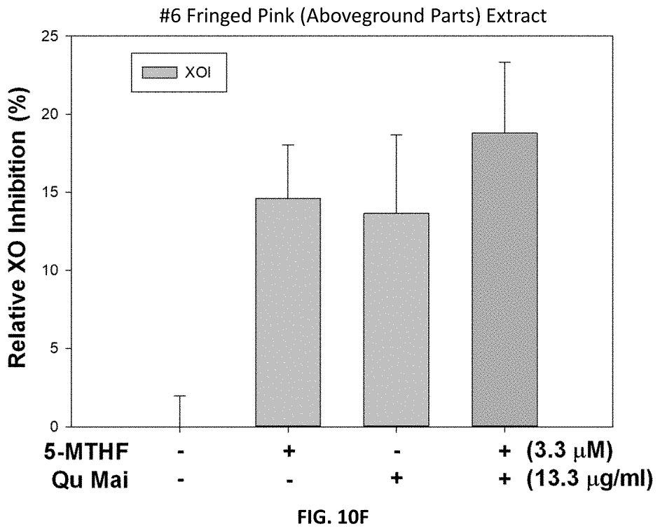

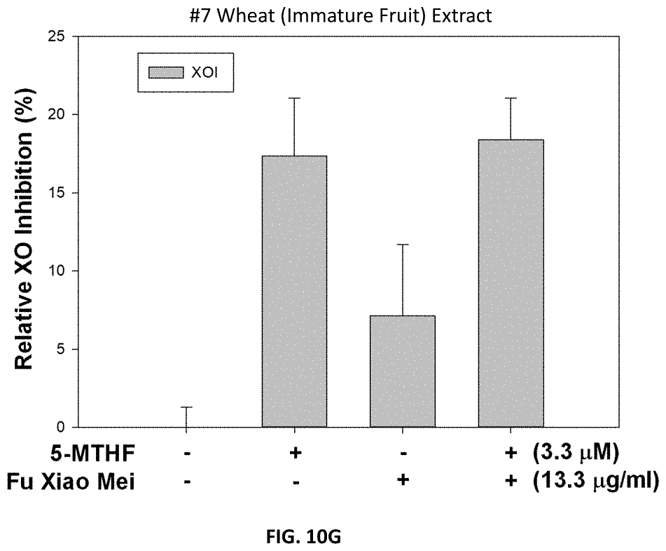

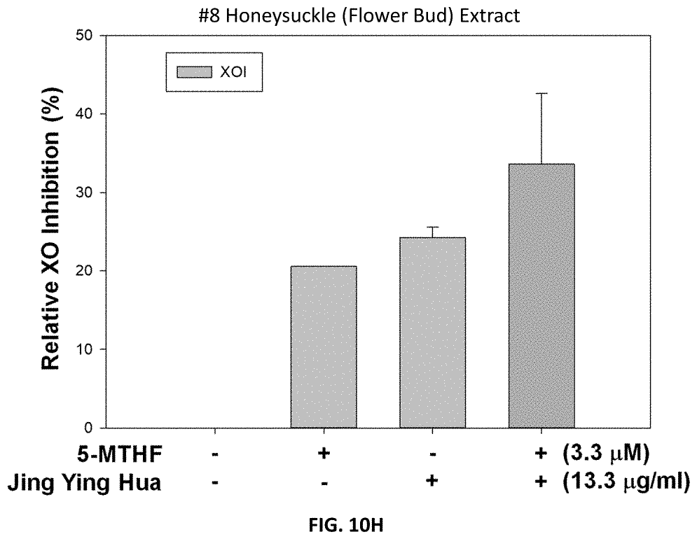

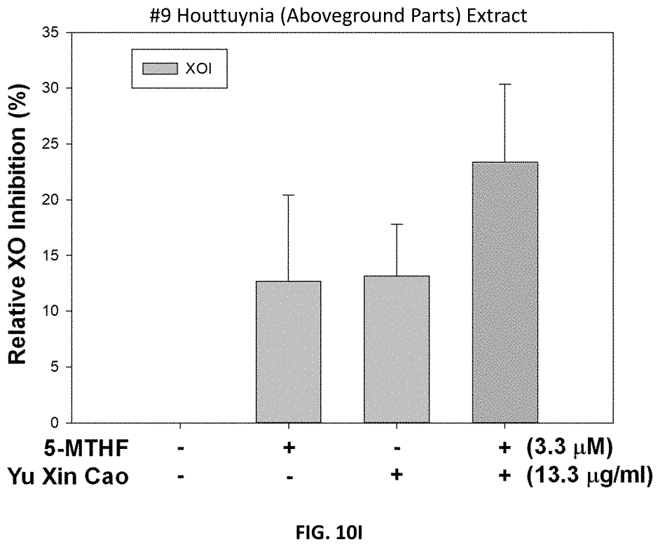

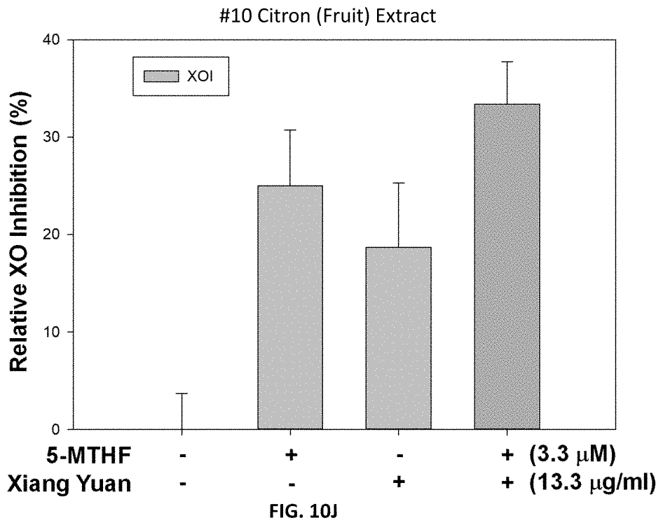

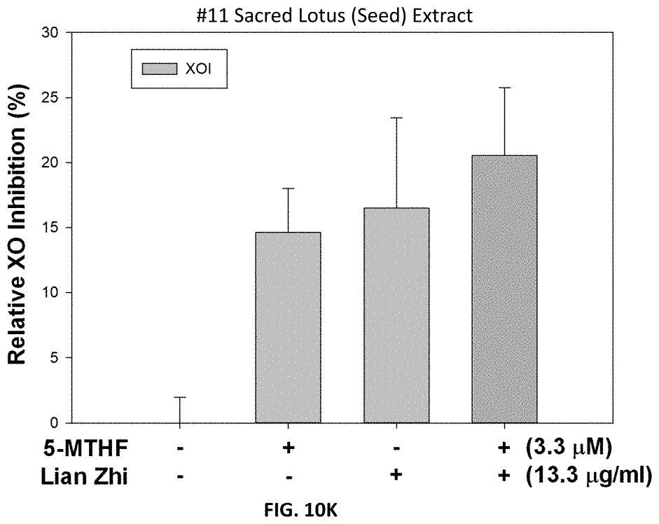

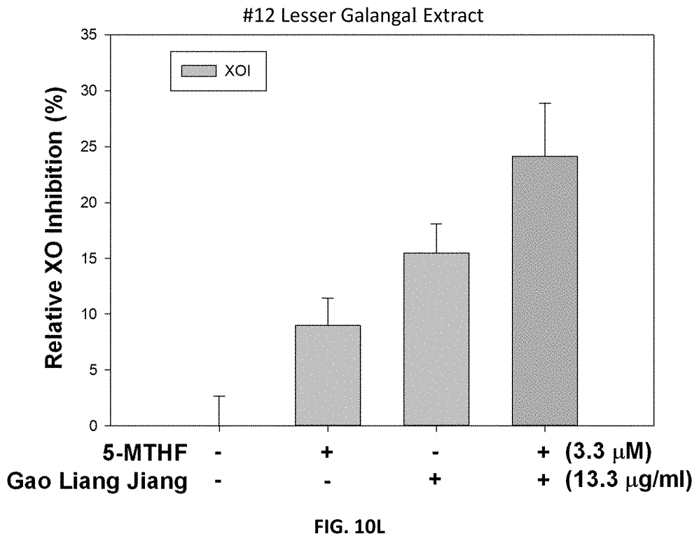

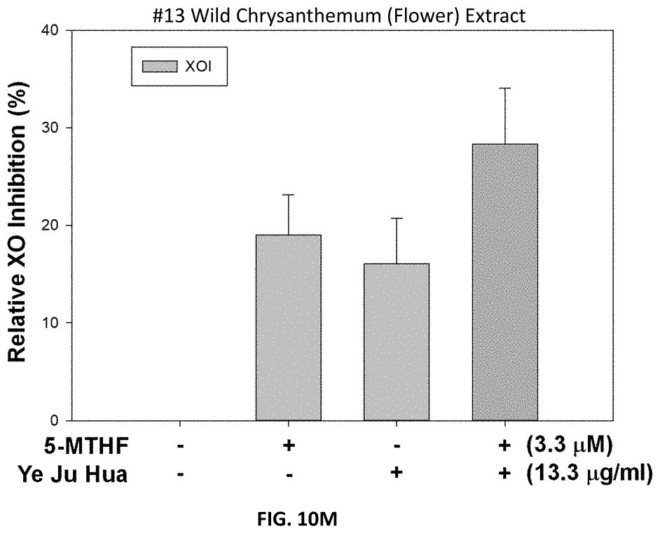

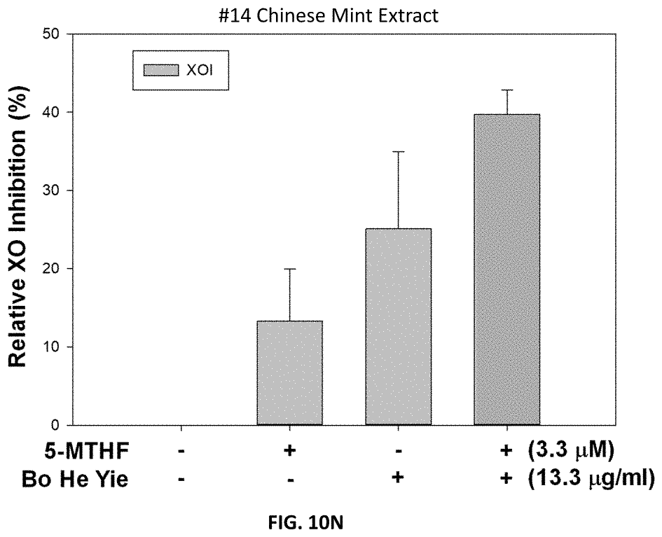

5-methyl tetrahydrofolate (5-MTHF) is considered a superior form of folic acid supplementing. Studies have shown that 5-MTHF can effectively inhibit XO activity in vitro and in vivo (90). The inventors have confirmed that 5-MTHF effectively inhibited XO activity in vitro in a concentration-dependent manner (FIG. 9). IC₅₀ for 5-MTHF is about 35.6 uM. However, the XO inhibitory effect of 5-MTHF is weaker than folic acid (IC₅₀ 0.75 uM). The inventors also tested the combination of 5-MTHF with different traditional Chinese medicine herb extracts for their effect on XO activity in vitro (FIG. 10). 5-MTHF had an additive effect on XO inhibition with 10 out of 14 herb extracts: Flowering Quince (Fruit) Extract; Euryale (Seed) Extract; Fringed Pink (Aboveground Parts) Extract: Honeysuckle (Flower Bud) Extract; Houttuynia (Aboveground Parts); Citron (Fruit) Extract; Sacred Lotus (Seed) Extract; Lesser Galangal Extract; Wild Chrysanthemum (Flower) Extract; and Chinese mint (FIG. 10), but not with Clove (Flower Bud) Extract; Vietnamese Sophora (Root) Extract; Rice Bean Extract; and Wheat (Immature Fruit) Extract.

In some embodiments, pre-mixing is utilized of folic acid or 5-methyl tetrahydrofolate and individual extracts on XO inhibition. After completion of studies of combinations of single extracts with folic acid, one can further characterize the combination of multiple herb extracts with folic acid. One can also characterize the combination of multiple herb extracts without folic acid and/or 5-methyl tetrahydrofolate for the XO inhibition.

One can extend XO activity assay to more Chinese herb extracts and other botanical ingredients and study their additive or synergistic effect among these agents with or without folic acid and/or 5-methyl tetrahydrofolate. Regarding XO inhibition studies, one can screen and characterize a few lead combination recipes of herb extracts with or without folic acid or 5-MTHF for animal model tests and ultimately for the treatment and/prevention of patients with both symptomatic and asymptomatically hyperuricemia conditions, which are a significant and independent risk factor for cardiovascular disease and many other diseases.

### Example 3

### Particular Embodiments of Dietary Health Supplements/Antioxidants for Controlling Hyperuricemia and Oxidative Stress

Embodiments of the disclosure encompass compositions (such as health food supplements or functional food) that are able to reduce blood uric acid, to reduce oxidative stress, and/or to lower the risks of gout and cardiovascular disease, for example. In such embodiments, one can select, characterize, optimize, synergize and formulate extracts of certain Chinese herbs and/or regular food items as well as vitamins, minerals and nutrients for the purpose of developing new, safe health food supplements or functional food for controlling hyperuricemia and oxidative stress, especially in its long-term use for the prevention and treatment of gout and hyperuricemia-induced cardiovascular disease. One can utilize a cell free system of XO enzyme activity assay and mouse models for this embodiment, for example.

One can employ in vitro screening of effective raw materials of dietary health supplement products and develop more effective recipes/formulas for the inhibition of xanthine oxdase (XO) activity and oxidative stress (a variety of substrates and combinations, additive effects, synergistic effects). One can use acute (short-term) animal studies to characterize the effectiveness and toxicity of dietary health supplement recipes/formulas for controlling hyperuricemia. One can also use chronic (relatively long-term) animal studies to characterize the effectiveness and toxicity of dietary health supplement recipes/formulas for controlling hyperuricemia.

In specific embodiments, some traditional Chinese herb medicines have potent XO-inhibitory activities as well as antioxidant activities; individual herb activities may have additive or synergistic effects with other herb medicines and/or with folic acid and/or one or more of its derivatives, for example (folic acid and its derivatives are XO inhibitors (52,53). In specific aspects, the functional food/dietary supplement formulation(s) of the present disclosure including traditional Chinese herb extract and (in some cases) folic acid and/or one or more of its derivatives provide a new preventative and/or treatment for asymptomatic hyperuricemia is utilized as a preventative and/or treatment. In specific cases, the compositions of the disclosure provide for long-term management of asymptomatic hyperuricemia, an independent risk factor for CVD, and therefore impacts CVD prevention.

Specific embodiments of the disclosure provide for confirmation of the efficacy of folic acid or 5-MTHF on control of hyperuricemia and may be included with traditional Chinese herb medicines and/or other botanical ingredients as dietary health supplements, for example for the long term management of hyperuricemia. Folic acid may be one of components, and it may enhance the therapeutic efficacy of the combination therapy.

Examples of herbal extracts that may be included in the compositions include one or more of the following:

TABLE 3
Herbal Extracts
Name
Acai Berry Extract Powder
African Mango Extract Powder
Ajuga Turkest Extract Powder
Alfalfa Extract Powder
Aloe Vera Extract Powder
American Ginseng Extract Powder
Amla Extract Powder
Andrographis Extract Powder
Aronia Extract Powder
Artichoke Extract Powder
Ashwagandha Extract Powder
Astragalus Extract Powder
Bamboo Extract Powder
Barley Extract Powder
Bearberry Leaf Extract (Uva Ursi) Powder
Bilberry Extract Powder
Bitter Melon Extract Powder
Black Cohosh Extract Powder
Black Garlic Extract Powder
Blood Orange Extract Powder
Boswellia Serrata Extract Powder
Broccoli Extract Powder
Buckthorn Bark Extract Powder
Burdock Root Extract Powder
Butcher's Broom Extract Powder
Butterbur Extract Powder
Caralluma Extract Powder
Cascara Sagrada Extract Powder
Cassia Seed Extract Powder
Cat's Claw Extract Powder
Catuaba Extract Powder
Cayenne Extract Capsules
Cayenne Extract Powder
Celery Seed Extract Powder
Chasteberry Extract Powder
Chitosan Extract Powder
Cinnamon Bark Extract Capsules
Cinnamon Bark Extract Powder
Cnidium Fruit Extract Powder
Cocoa Extract Powder
Coriolus Versicolor Extract (Turkey Tail) Powder
Corn Silk Extract Powder
Corydalis Extract Powder
Creek Yellow Grass Extract Powder
Curcumin 95% Natural Turmeric Extract Powder
Dandelion Root Extract Powder
Devil's Claw Extract Powder
Dragon Fruit Extract Powder
Echinacea Extract Powder
Elderberry Extract Powder
Eyebright Extract Powder
Flaxseed Extract Powder
Fo-Ti Extract Powder
Garlic Extract Powder
Ginger Root Extract Powder
Ginkgo Biloba Leaf Extract Powder
Ginseng Root Extract Powder
Grape Seed Extract Powder
Grapefruit Seed Extract Powder
Green Tea Extract (50% EGCG) Powder
Guanabana Extract Powder
Gymnema Extract Powder
Gynostemma Extract Powder
Hawthorn Berry Extract Powder
Hawthorn Leaf Extract Powder
Hibiscus Flower Extract Powder
Hoodia Extract Powder
Hops Extract Powder
Horse Chestnut Extract Powder
Horsetail Extract Powder
Kava Kava Extract Powder
Kelp Extract Powder
Kola Nut Extract Powder
Kudzu Root Extract Powder
Lemon Balm Extract Powder
Licorice Root Extract Powder
LongJack Extract (Tongkat Ali) Powder
Maca Root Extract Powder
Magnolia Extract Powder
Marshmallow Root Extract Powder
Milk Thistle Extract Powder
Moringa Extract Powder
Motherwort Extract Powder
Mucuna Pruriens Extract Powder
Muira Puama Extract Powder
Nettle Extract Powder
Oat Straw Extract Powder
Olive Leaf Extract Powder
Oolong Tea Extract Powder
Oyster Extract Powder
Papaya Fruit Extract Powder
Parsley Extract Powder
Passion Flower Extract Powder
Peppermint Extract Powder
Pine Bark Extract Powder
Pomegranate Extract Powder-(40% Ellagic Acid)
Portulaca Oleracea (Purslane) Extract Powder
Pumpkin Seed Extract Powder
Red Clover Extract Powder
Red Yeast Rice Extract (RYR) Powder
Reishi Mushroom Extract Powder
Rosemary Extract Powder
Sage Extract Powder
Saw Palmetto Extract Powder
Senna Leaf Extract Powder
Slippery Elm Bark Extract Powder
Spearmint Extract Powder
Spinach Extract Powder
St John's Wort Extract Powder
Tart Cherry Extract Powder
Taxillus Chinensis Danser Extract Powder
Turkey Rhubarb Extract Powder
Valerian Root Extract Powder
White Mulberry Fruit Extract Powder
White Willow Bark Extract 15% Capsules
White Willow Bark Extract Powder
Wild Yam Extract Powder
Yohimbe Extract Powder

In addition, one or more of the following compositions may be utilized in any composition(s) in the disclosure. These vitamins may have additive or synergistic effects with composition(s) of the disclosure, such as for controlling hyperuricemia, oxidative stress and/or hyperhomocysteinemia, for example, which are risk factors for cardiovascular diseases and many other diseases. Therefore, any composition of the disclosure, including those that employ the following, may be utilized as a preventative composition.

TABLE 4
Selected vitamin supplements
Names
Vitamin A
Vitamin D3
Vitamin B1
Vitamin B2
Vitamin B6
Vitamin B12
Nicotinic acid (B3)
Folic acid (B9)
Methyl Folate (5-MTHF)
Biotin (B7)
Choline
Vitamin C
Vitamin K
Pantothenic acid
Vitamin E

The following items in Table 5 may be utilized in any composition(s) of the present disclosure.

TABLE 5
Additional herb extracts
Description
Extract 5:1 100 g | Morinda
Extract 5:1 100 g | Tribulus (Fruit)
Extract 5:1 100 g | Chinese Peony
Extract 10:1 100 g | Bai-Zhu Atractylodes (Rhizome)
Extract 10:1 100 g | Platycladi Seed
Extract 5:1 100 g | Psoraleae
Extract 10:1 100 g | Oriental Arborvitae (Leaf & Branch Tip)
Extract 5:1 100 g | Cang-Zhu Atractylodes (Rhizome)
Extract 5:1 | Asian Plantain
Extract 5:1 100 g | Asian Plantain (Seed)
Extract 5:1 100 g | Chinese Peony
Extract 5:1 100 g | Cyathula
Extract Extract 5:1 100 g | Sichuan Lovage
Extract 5:1 100 g | Siberian Ginseng
Extract 5:1 100 g | Chinese Salvia (Root & Rhizome)
Extract 5:1 100 g | Radix Angelicae Sinensis
Extract 5:1 100 g | Radix Codonopsis
Extract 5:1 100 g | Lycium
Extract 5:1 100 g | Eucommia
Extract 5:1 100 g | Senna (Leaf)
Extract 5:1 100 g | Drynaria (Rhizome)
Extract 10:1 100 g | Rhodiola rosea linn
Extract 5:1 100 g | Common Fenugreek Seed
Extract 5:1 100 g | Achyranthes (Root)
Extract 10:1 100 g | Astragalus (Root)
Extract 5:1 100 g | Dogbane Leaf
Extract 5:1 100 g | Rugose Rose (Flower Bud)
Extract 5:1 100 g | Tree Peony (Root Bark)
Extract 5:1 100 g | Radix Aucklandiae
Extract 5:1 100 g | Burdock (Fruit)
Extract 5:1 100 g | Chinese Eupatorium (Aboveground Parts)
Extract 5:1 100 g | Cattail (Pollen)
Extract 5:1 100 g | Indian Madder (Root & Rhizome)
Extract 5:1 100 g | Chinese Ash (Stem Bark)
Extract 5:1 100 g | Glehnia (Root)
Extract 5:1 100 g | Flatstem Milkvetch Seed
Extract 100 g | Asiatic Dogwood (Fruit without Seed)
Extract 5:1 100 g | Chinese Cimicifuga (Rhizome)
Extract 5:1 100 g | Pseudostellaria (Root Tuber)
Extract 5:1 100 g | Gastrodia (Rhizome)
Extract 5:1 100 g | Chinese Asparagus (Root Tuber)
Extract 5:1 100 g | Cuscutae Semen
Extract 5:1 100 g | Schisandra (Fruit)
Extract 5:1 100 g | Heal All (Fruit Spike)
Extract 5:1 100 g | Scrophularia (Root)
Extract 5:1 | Rhizoma Cyperi/Nutgrass Galingale Rhizome
Extract 5:1 100 g | Chinese Motherwort (Aboveground Parts)
Extract 5:1 100 g | Epimedium (Aboveground Parts)
Extract 10:1 100 g | Radix Polygalae
Extract 5:1 100 g | Asian Water Plantain (Rhizome)
Extract 5: 100 g | Bulbus Fritillariae Thunbergii
Extract 10:1 100 g | Nacre (Calcined)
Extract 5:1 100 g | Processed Fo-Ti (Cured Root Tuber)
Extract 5:1 100 g | Anemarrhena (Rhizome)
Extract 5:1 100 g | Ligustrum (Fruit, Cured)
Extract 5:1 100 g | Caulis Bambusae In Taenia

An example of an experimental design is as follows. For initial screening individual extracts for XO inhibitory and antioxidant effects, one may employ any suitable method include at least the following:

Method (1):

Xanthine oxidase (XO) inhibition assay (using one or more substrate concentrations for screening). The detailed method is described in a previous publication (132).

Method (2):

DPPH (2,2-diphenyl-1-picrylhydrazyl) scavenging assay (using one substrate concentration for screening). The abilities of the tested compounds to scavenge DPPH radicals are measured optically by monitoring the decrease of the absorption at 429 nm. The compounds' antioxidant activities are compared to those of vitamin C and vitamin E. The detailed method is described in our previous publication (132).

One may search and develop effective recipes/formulas based on selected effective health supplement raw materials (a variety of substrates and combinations, additive effects, synergistic effects can be performed). If initial screening results from raw materials identify a group of extracts that inhibit XO activity, one can utilize the effective standard extracts for further characterization studies. The concentration range of these selected materials is expanded and mixed with other materials. In some cases, effects of different combinations of individual herb extract with folic acid, 5-MTHF or other herb extracts on XO inhibition and antioxidant potential may be tested in in vitro assays. Any additive or synergistic effects of these combinations is characterized. If additive or synergistic effects of components exist, one can utilize such component(s) for combinational formulations (for example, with small amounts of each components in the combination) to achieve the maximal therapeutic effects, while reducing potential side effects of each component and reducing the potential risk of drug resistance for long-term management of hyperuricemia, in at least some cases.

Use of Acute (Short-Term) Animal Experiments to Characterize the Effectiveness and Toxicity of Dietary Health Supplement Recipes/Formulas for Controlling Hyperuricemia

Materials for Studies:

According to the results of in vitro experiments, effective recipes/formulas of combinational herb extracts with or without folic acid/5-MTHF are characterized in animal studies. In specific embodiments, each formulation may contain 3 to 5 individual extracts or natural compounds/vitamin supplements. One may or may not test individual ingredients in animal experiments prior to testing them in the combination formulations in animal experiments. In specific embodiments, only recipes/formulas as a whole are characterized in animal experiments.

Example of a Study Design:

A. Formulation and Characterization of Selected Combination Recipes for Animal Studies

Based on initial studies, DMSO-soluble factions of herb extracts had stronger XO inhibitory effects than their water-soluble fractions. DMSO dissolves both polar and non-polar compounds in the extract. DMSO is effective for getting a wide range of compounds into solution. Each herb extract selected from initial studies are further extracted in DMSO and freeze-dried (lyophilized). XO inhibitory effect and antioxidant potential of each DMSO extract are reconfirmed by in vitro XO activity assay and DPPH scavenging assay, respectively. One or more oils, such as one or more cooking oils (for example, oleic acid, isopropyl myristate, medium chain triglyceride, olive oil, castor oil, peanut oil, corn oil, sesame oil, soybean oil, almond oil, linseed oil, rapeseed oil, sunflower oil, coconut oil, groundnut oil, and/or palm oil) may be selected to mix these extracts for animal administration (oral gavage).

B. Acute Model of Hyperuricemia and Toxicity in Mice

One can determine the hypouricemic effect of the formulation in allantoxanamide-treated mice. Intraperitoneal (i.p.) injection of uricase inhibitor allantoxanamide can effectively block the conversion of uric acid to 5-hydroxyisourate and thus cause a marked increase in serum uric acid levels in mice, providing a hyperuricemic animal model. One can characterize the hypouricemic effect of selected formulations in a well characterized mouse model (Table 6). Selected formulations will administered to the mouse by oral gavage. The detailed method is described in a previous publication (132).

TABLE 6
Examples of animal assignments for studying the
hypouricemic effect of XO inhibitors

| Group | | | |
|---|---|---|---|
| (n = 10) | Allantoxanamide | Treatment | End Point |
| 1 | − | PEG400 (Control) | Serum uric acid |
| 2 | + | PEG400 (Control) | Serum uric acid |
| 3 | + | Formulation 1, low dose | Serum uric acid |
| 4 | + | Formulation 1, high dose | Serum uric acid |
| 5 | + | Formulation 2, low dose | Serum uric acid |
| 6 | + | Formulation 2, high dose | Serum uric acid |
| 7 | + | Allopurinol, 50 mg/kg | Serum uric acid |
| 8 | + | Allopurinol, 100 mg/kg | Serum uric acid |

Toxicity in mice. One can investigate the potential toxicity of selected formulations in mice, focusing on low dose and high dose (oral administration). Negative control and Allopurinol control may be included. Formulated health supplements may be administrated into mice by oral garage once a day for 15 days. Major end points include general health conditions and body weight, blood counts, blood chemistry and enzymes (including liver, heart and kidney functional panels), and organ histology (Table 7).

TABLE 7
Animal assignments for studying the potential toxicity of XO inhibitors

| Group | | | | |
|---|---|---|---|---|
| (n = 10) | Treatment | Frequency | Sacrifice | End Points |
| 1 | PEG400 (Control) | Once daily | 15 days | Mortality |
| 2 | Formulation 1, | Once daily | 15 days | General health |
| | low dose | | | body weight |
| 3 | Formulation 1, | Once daily | 15 days | Blood counts |
| | high dose | | | Blood chemistry |
| 4 | Formulation 2, | Once daily | 15 days | Organ histology |
| | low dose | | | |
| 5 | Formulation 2, | One time only | 15 days | |
| | high dose | | | |
| 6 | Allopurinol, | One time only | 15 days | |
| | 50 mg/kg | | | |
| 7 | Allopurinol, | One time only | 15 days | |
| | 100 mg/kg | | | |

Use of Chronic (Relatively Long-Term) Animal Experiments to Test the Effectiveness and Toxicity of Dietary Health Supplement Recipes/Formulas for Controlling Hyperuricemia

Study Design:

Chronic model of hyperuricemia in uricase knockout mice: In mice and many other mammals, normal blood levels of uric acid are relatively low (1-2 mg/dL) because uric acid is further oxidized to allantoin by uricase (Chen, Lu, et al., 2016). The loss of uricase in humans and higher primates occurred about 15 million years ago, resulting in a relatively higher serum uric acid (SUA) level than that in these lower animals. In order to establish an animal model of chronic hyperuricemia, scientists have created uricase (Uox) gene knockout mice that show severe hyperuricemia and urate nephropathy (133). Under the maintenance of Allopurinol, adult uricase−/− mice show relatively low SUA (2-4 mg/dL). However, once Allopurinol is discontinued, uricase−/− mice will have high SUA levels (6-10 mg/dL) in one week. The long-term hypouricemia effect of selected formulations can be determined by using uricase knockout mice. Selected formulations will be administrated through oral gavage once a day for 2 weeks or through drinking water for 2 months. Serum uric acid level will be tested every two days or once a week. During the study, general health conditions including body weight will be monitored. Blood counts, blood chemistry and enzymes (including liver, heart and kidney functional panels), and organ histology will be studied when the mice will be sacrificed (Table 6). It is estimated that about 500 g of each material would be needed for the current experiments. Each herb extract selected from PLAN I study will be further extracted in DMSO and freeze-dried. XO inhibitory effect and antioxidant potential of each DMSO extract will be reconfirmed by in vitro XO activity assay and DPPH scavenging assay, respectively. One or more oils, such as one or more cooking oils (for example, oleic acid, isopropyl myristate, medium chain triglyceride, olive oil, castor oil, peanut oil, corn oil, sesame oil, soybean oil, almond oil, linseed oil, rapeseed oil, sunflower oil, coconut oil, groundnut oil, and/or palm oil) may be selected to mix these extracts for animal administration (oral gavage).

TABLE 8
Uricase−/− mice assignments for studying hypouricemic effect and the
potential toxicity of XO inhibitors

| Group | Treatment | | | |
|---|---|---|---|---|
| (n = 10) | (10 mg/kg) | Frequency | Sacrifice | End Points |
| 1 | Oral gavage, | Once daily | 15 days | Mortality Serum |
| | PEG400 | | | uric acid |
| 2 | Oral gavage | Once daily | 15 days | General health |
| 3 | Oral gavage | Once daily | 15 days | conditions |
| 4 | Drinking water | Continuously | 60 days | and body weight |
| | (Control) | | | Blood counts Blood |
| 5 | Drinking water, | Continuously | 60 days | chemistry Organ |
| 6 | Drinking water, | Continuously | 60 days | histology |

All publications mentioned in the specification are indicative of the level of those skilled in the art to which the disclosure pertains. All publications are herein incorporated by reference to the same extent as if each individual publication was specifically and individually indicated to be incorporated by reference.

### REFERENCES FOR EXAMPLES 1-3

1. Benjamin E J, Virani S S, Callaway C W, Chamberlain A M, et al. American Heart Association Council on Epidemiology and Prevention Statistics Committee and Stroke Statistics Subcommittee. Heart Disease and Stroke Statistics-2018 Update: A Report From the American Heart Association. Circulation. 2018 Mar. 20; 137(12):e67-e492.
2. Galassi F M, Borghi C. A brief history of uric acid: from gout to cardiovascular risk factor. Eur J Intern Med. 2015; 26:373.
3. Borghi C, Desideri G. Urate-lowering drugs and prevention of cardiovascular disease: The emerging role of xanthine oxidase inhibition. Hypertension, 2016; 67(3): 496-98
4. Li M, Hu X, Fan Y et al: Hyperuricemia and the risk for coronary heart disease morbidity and mortality a systematic review and dose-response meta-analysis. Sci Rep, 2016; 6: 19520
5. Storhaug H M, Norvik J V, Toft I et al: Uric acid is a risk factor for ischemic stroke and all-cause mortality in the general population: A gender specific analysis from The Tromso Study. BMC Cardiovasc Disord, 2013; 13: 115
6. Tian Zuo, Xuehui Liu et al. Hyperuricemia and coronary heart disease mortality: a meta-analysis of prospective cohort studies. BMC Cardiovascular Disorders, 2016: 16:207.
7. Loeffler L F, Navas-Acien A, Brady T_{M}, et al. Uric acid level and elevated blood pressure in US adolescents: national health and nutrition examination survey, 1999-2006. Hypertension, 2012, 59(4): 811-817.
8. Fan Y, Wei F, Lang Y, et al. Losartan treatment for hypertensive patients with hyperuricaemia in Chinese population: a meta-analysis. J Hypertens, 2015, 33(4): 681-688; discussion 689.
9. Kuwabara M, Niwa K, Hisatome I. Hyperuricemia is an independent risk factor of atrial fibrillation. Journal of the American College of Cardiology 2014:63(12) Supplement. DOI: 10.1016/S0735-1097(14)60469-1
10. Alderman M H: Serum uric acid as a cardiovascular risk factor for heart disease. Curr Hypertens Rep 2001; 3: 184-189.
11. Kuwabara M, Niwa K, Nishi Y, et al: Relationship between serum uric acid levels and hypertension among Japanese individuals not treated for hyperuricemia and hypertension. Hypertens Res 2014; 37: 785-789.
12. Grayson P C, et al. Hyperuricemia and incident hypertension: a systematic review and meta-analysis. Arthritis Care Res (Hoboken). 2011; 63(1):102-10.
13. Mazzali M, Hughes J, Kim Y G, et al. Elevated uric acid increases blood pressure in the rat by a novel crystal-independent mechanism. Hypertension, 2001, 38(5): 1101-1106.
14. Biscaglia S, Ceconi C, Malagil M, et al. Uric acid and coronary artery disease: an elusive link deserving further attention. Int J Cardiol, 2016, 213: 28-32.
15. Lv Q, et al. High serum uric acid and increased risk of type 2 diabetes: a systemic review andmeta-analysis of prospective cohort studies. PLoS One. 2013; 8(2):e56864.
16. Zhu Y, et at. Prevalence of gout and hyperuricemia in the U.S. general population: The National Health and Nutrition Examination Survey 2007-2008. Arthritis Rheum. 2011, 63, 3136-3141.
17. Liu R, Han C, Wu D et al: Prevalence of hyperuricemia and gout in mainland China from 2000 to 2014: A systematic review and meta-analysis. Biomed Res Int, 2015; 2015: 762820
18. Smith E, et al. Global Prevalence of Hyperuricemia: A Systematic Review of Population-Based Epidemiological Studies. Arthritis Rheumatol. 2015; 67 (suppl 10).
19. Chou C T, Lai J S. The epidemiology of hyperuricaemia and gout in Taiwan aborigines. Br J Rheumatol, 1998; 37(3):258-62.
20. Chen C, Lu J M, Yao Q. Hyperuricemia-Related Diseases and Xanthine Oxidoreductase (XOR) Inhibitors: An Overview. Med Sci Monit. 2016 Jul. 17; 22:2501-12.
21. MacIsaac R L, Salatzki J, Higgins P, Walters M R, Padmanabhan S, Dominiczak A F, et al. Allopurinol and cardiovascular outcomes in adults with hypertension. Hypertension 2016; 67, 535-540.
22. Chen J H, Lan J L, Cheng C F, et al. Effect of urate-lowering therapy on the risk of cardiovascular disease and all-cause mortality in patients with gout: a case-matched cohort study. J Rheumatol. 2015; 42(9):1694-701.
23. Chen J H, Lan J L, Cheng C F, et al. Effect of urate-lowering therapy on all-cause and cardiovascular mortality in hyperuricemic patients without gout: a case-matched cohort study. PLoS One. 2015; 10(12):e0145193.
24. Luk A J, Levin G P, Moore E E, et al. Allopurinol and mortality in hyperuricaemic patients. Rheumatology (Oxford) 2009; 48:804-6
25. Dubreuil M, Zhu Y, Zhang Y, et al. Allopurinol initiation and all cause mortality in the general population. Ann Rheum Dis 2014; 74:1368-7.
26. Sluijs I, Beulens J W, van der A D L, Spijkerman A M, Schulze M B, van der Schouw Y T. Plasma uric acid is associated with increased risk of type 2 diabetes independent of diet and metabolic risk factors. J Nutr. 2013 January; 143(1):80-5. doi: 10.3945/jn.112.167221. Epub 2012 Nov. 21.
76. Krishnan E, Pandya B J, Chung L, Hariri A, Dabbous O. Hyperuricemia in young adults and risk of insulin resistance, prediabetes, and diabetes: a 15-year follow-up study. Am J Epidemiol. 2012 Jul. 15; 176(2):108-16. doi: 10.1093/aje/kws002. Epub 2012 Jul. 2.
28. Li C, Hsieh M C, Chang S J. Metabolic syndrome, diabetes, and hyperuricemia. Curr Opin Rheumatol. 2013 March; 25(2):210-6. doi: 10.1097/BOR.0b013e32835d951e. Review.
29. Chang H Y, Tung C W, Lee P H, Lei C C, Hsu Y C, Chang H H, Yang H F, Lu L C, Jong M C, Chen C Y, Fang K Y, Chao Y S, Shih Y H, Lin C L. Hyperuricemia as an independent risk factor of chronic kidney disease in middle-aged and elderly population. Am J Med Sci. 2010 June; 339(6):509-15. doi: 10.1097/MAJ.0b013e3181db6e16.
30. Tsuruta N, Imafuku S, Narisawa Y. Hyperuricemia is an independent risk factor for psoriatic arthritis in psoriatic patients. J Dermatol. 2017 December; 44(12):1349-1352. doi: 10.1111/1346-8138.13968. Epub 2017 Jul. 10.
31. Chang H-Y, Lee P-H, Lei C-C, Tung C-W, Hsu Y-C, Huang T-J, et al. (2013) Hyperuricemia Is an Independent Risk Factor for New Onset Micro-Albuminuria in a Middle-Aged and Elderly Population: A Prospective Cohort Study in Taiwan. PLoS ONE 8(4): e61450. https://doi.org/10.1371/journal.pone.0061450
32. Long H, Jiang J, Xia J, Jiang R, He Y, Lin H, Fan Z, Zeng T. Hyperuricemia Is an Independent Risk Factor for Erectile Dysfunction. J Sex Med. 2016 July; 13(7):1056-62. doi: 10.1016/j.jsxm.2016.04.073. Epub 2016 May 18.
33. Doherty A, Carvalho J C, Drewlo S, El-Khuffash A, Downey K, Dodds M, Kingdom J. Altered hemodynamics and hyperuricemia accompany an elevated sFlt-1/P1GF ratio before the onset of early severe preeclampsia. J Obstet Gynaecol Can. 2014 August; 36(8):692-700. doi: 10.1016/S1701-2163(15)30511-9.
34. Springer J, Tschirner A, Hartman K, Palus S, Wirth E K, Ruis S B, Möller N, von Haehling S, Argiles J M, Kohrle J, Adams V, Anker S D, Doehner W. Inhibition of xanthine oxidase reduces wasting and improves outcome in a rat model of cancer cachexia. Int J Cancer. 2012 Nov. 1; 131(9):2187-96. doi: 10.1002/ijc.27494. Epub 2012 Jul. 9.
35. Zhou F L, Zhang W G, Wei Y C, Meng S, Bai G G, Wang B Y, Yang H Y, Tian W, Meng X, Zhang H, Chen S P. Involvement of oxidative stress in the relapse of acute myeloid leukemia. J Biol Chem. 2010 May 14; 285(20):15010-5. doi: 10.1074/jbc.M110.103713. Epub 2010 Mar. 16.
36. Honorat J A, Kinoshita M, Okuno T, Takata K, Koda T, Tada S, Shirakura T, Fujimura H, Mochizuki H, Sakoda S, Nakatsuji Y. Xanthine oxidase mediates axonal and myelin loss in a murine model of multiple sclerosis. PLoS One. 2013 Aug. 8; 8(8):e71329. doi: 10.1371/journal.pone.0071329. eCollection 2013.
37. Peglow S, Toledo A H, Anaya-Prado R, Lopez-Neblina F, Toledo-Pereyra L H. Allopurinol and xanthine oxidase inhibition in liver ischemia reperfusion. J Hepatobiliary Pancreat Sci. 2011 March; 18(2):137-46. doi: 10.1007/s00534-010-0328-7. Review.
38. Kim N H, Choi S, Han E J, Hong B K, Choi S Y, Kwon H M, Hwang S Y, Cho C S, Kim W U. The xanthine oxidase-NFAT5 pathway regulates macrophage activation and TLR-induced inflammatory arthritis. Eur J Immunol. 2014 September; 44(9):2721-36. doi: 10.1002/eji.201343669. Epub 2014 Aug. 11.
39. Fang J, Yin H, Liao L, Qin H, Ueda F, Uemura K, Eguchi K, Bharate G Y, Maeda H. Water soluble PEG-conjugate of xanthine oxidase inhibitor, PEG-AHPP micelles, as a novel therapeutic for ROS related inflammatory bowel diseases. J Control Release. 2016 Feb. 10; 223:188-196. doi: 10.1016/j.jconre1.2015.12.049. Epub 2015 Dec. 29.
40. CHMP Assessment Report For Adenuric (International Nonproprietary Name: Febuxostat), Procedure No. EMEA/H/C/777, European Medicines Agency (EMEA) Evaluation of Medicines for Human Use. EMEA/258531/2008
41. Bohm M, Vuppalanchi R, Chalasani N, Drug-Induced Liver Injury Network (DILIN): Febuxostat-induced acute liver injury. Hepatology, 2016; 63(3): 1047-49
42. Reinders M K, Jansen T L (2010) Management of hyperuricemia in gout: focus on febuxostat. Clinlntery Aging, 2010; 5: 7-18.
43. Gray C L, Walters-Smith N E (2011). Febuxostat for treatment of chronic gout. Am J Health Syst Pharm 2011; 68: 389-398.
44. Chen C, Lu J M, Yao Q. Hyperuricemia-Related Diseases and Xanthine Oxidoreductase (XOR) Inhibitors: An Overview. Med Sci Monit. 2016 Jul. 17; 22:2501-12.
45. Chohan S: Safety and efficacy of febuxostat treatment in subjects with gout and severe allopurinol adverse reactions. J Rheumatol, 2011; 38: 1957-59
46. Abeles A M: Febuxostat hypersensitivity. J Rheumatol, 2012; 39: 659
47. Schumacher H R Jr., Becker M A, Wortmann R L et al: Effects of febuxostat versus allopurinol and placebo in reducing serum urate in subjects with hyperuricemia and gout: A 28-week, phase III, randomized, double-blind, parallel-group trial. Arthritis Rheum, 2008; 59(11): 1540-48
48. www.accessdata.fda.gov. Accessed Apr. 27, 2018
49. Mauck M, Taintor A, Jha P: Cross-sensitivity of allopurinol and febuxostat-induced drug rash with eosinophilia and systemic symptoms (DRESS) syndrome. J Gen Intern Med, 2010; 25: S504-5
50. Bardin T, Chales G, Pascart T et al: Risk of cutaneous adverse events with febuxostat treatment in patients with skin reaction to allopurinol. A retrospective, hospital-based study of 101 patients with consecutive allopurinol and febuxostat treatment. Joint Bone Spine, 2016; 83(3): 314-17
51. Kang Y, Kim M J, Jang H N et al: Rhabdomyolysis associated with initiation of febuxostat therapy for hyperuricaemia in a patient with chronic kidney disease. J Clin Pharm Ther, 2014; 39(3): 328-30
52. Lewis A S, Murphy L, McCalla C, Fleary M, Purcell S. Inhibition of mammalian xanthine oxidase by folate compounds and amethopterin. J Biol Chem 1984; 259:12-5.
53. Williams J N Jr., Nichol C A, Elvehjem C A. Relation of dietary folic acid and vitamin B12 to enzyme activity in the chick. J Biol Chem 1949; 180:689-94.
54. Qin X, Li Y, He M, Tang G, Yin D, Liang M, Wang B, Nie J, Huo Y, Xu X, Hou F F. Folic acid therapy reduces serum uric acid in hypertensive patients: a substudy of the China Stroke Primary Prevention Trial (CSPPT). Am J Clin Nutr. 2017 April; 105(4):882-889.
55. Li H, Qin X, Xie D, Tang G, Zhang Y, Li J, Hou F, Wang X, Huo Y, Xu X. Effects of combined enalapril and folic acid therapy on the serum uric acid levels in hypertensive patients: a multicenter, randomized, double-blind, parallel-controlled clinical trial. Intern Med. 2015; 54(1):17-24.
56. Qin X, Xu M, Zhang Y, Li J, Xu X, Wang X, Xu X, Huo Y. Effect of folic acid supplementation on the progression of carotid intima-media thickness: a meta-analysis of randomized controlled trials. Atherosclerosis. 2012 June; 222(2):307-13.
57. Huo Y, Li J, Qin X, Huang Y, Wang X, Gottesman R F, Tang G, et al. Efficacy of folic acid therapy in primary prevention of stroke among adults with hypertension in China: the CSPPT randomized clinical trial. JAMA. 2015 Apr. 7; 313(13):1325-35.
58. Huo Y, Qin X, Wang J, Sun N, Zeng Q, Xu X, Liu L, Xu X, Wang X.

Efficacy of folic acid supplementation in stroke prevention: new insight from a meta-analysis. Int J Clin Pract. 2012 June; 66(6):544-51.
59. Li Y, Huang T, Zheng Y, Muka T, Troup J, Hu F B. Folic Acid Supplementation and the Risk of Cardiovascular Diseases: A Meta-Analysis of Randomized Controlled Trials. J Am Heart Assoc. 2016 Aug. 15; 5(8).
60. Horiuchi H, Ota M, Kobayashi M, Kaneko H, Kasahara Y, Nishimura S, Kondo S, Komoriya K. A comparative study on the hypouricemic activity and potency in renal xanthine calculus formation of two xanthine oxidase/xanthine dehydrogenase inhibitors: TEI-6720 and allopurinol in rats. Res Commun Mol Pathol Pharmacol. 1999; 104(3):307-19.
61.[Shang Yanjun, Li Yiming, Jiang Shanhao, et al. The effect of Acteoside in Scrophulariaceae on hyperuricemia in mice. Pharmaceutical Journal of Chinese People's Liberation Army, 2004, 22(1): 30 32].
62. Lopes Galeno D M, Carvalho R P, Boleti A P, Lima A S, Oliveira de Almeida P D, Pacheco C C, Pereira de Souza T, Lima E S. Extract from Eugenia punicifolia is an antioxidant and inhibits enzymes related to metabolic syndrome. Appl Biochem Biotechnol. 2014 January; 172(1):311-24.
63. Schmeda-Hirschmann G, Theoduloz C, Franco L, Ferro E, de Arias A R. Preliminary pharmacological studies on Eugenia uniflora leaves: xanthine oxidase inhibitory activity. J Ethnopharmacol. 1987 November; 21(2):183-6.
64. Suzuki R, Hasuike Y, Hirabayashi M, Fukuda T, Okada Y, Shirataki Y. Identification of a xanthine oxidase-inhibitory component from Sophora flavescens using NMR-based metabolomics. Nat Prod Commun. 2013 October; 8(10):1409-12.
65. Shuai Xuehong; Hu Tingjun; Zeng Yun; Li Yuehua; Liu Hongli; Su Zijie; He Defeng. Effects of Shandougen Polysaccharide on Immune Organ Index and Free Radical Related Enzyme Activities in Immunosuppressive Model Mice. Journal of Nanjing Agricultural University [ISSN:1000-2030/CN:32-1148/S] Volume: 32 Volumes Number of Issues: 2009 Issue 2 Page Number: 170-172.
66. Kong L D, Cai Y, Huang W W, Cheng C H, Tan R X. Inhibition of xanthine oxidase by some Chinese medicinal plants used to treat gout. J Ethnopharmacol. 2000 November; 73(1-2): 199-207.
67. Zhang Xue, Zhou Ying, Guan Jing, Yan Shouqun, Pi Tingting. Inhibition of xanthine oxidase by extracts of five Chinese medicinal materials. Journal of Mountain Agriculture and Biology; 2015, 04 (2015/12/11), P51-54.
68. Sun Weifeng, Luo Ping, Xu Wei, et al. The effect of Xiezhuo Chubi Decoction (Smilax glabra, Coix, Coix seed, Weilingxian, papaya, Eupatorium adenophorum, Wang Buliuxing) on blood uric acid in mice with hyperuricemia. Chinese Journal of Experimental Formulas, 2006, 12(9): 36-38.
69. Liu L, Shi S, Zhao H, Yu J, Jiang X, Chen X. Selective fishing and analysis of xanthine oxidase binders from two Fabaceae species by coupling enzyme functionalized core-shell magnetic nanoparticles with HPLC-MS. J Chromatogr B Analyt Technol Biomed Life Sci. 2014 Jan. 15; 945-946:163-70.
70. Hudaib M M, Tawaha K A, Mohammad M K, Assaf A M, Issa A Y, Alali F Q, Aburjai T A, Bustanji Y K. Xanthine oxidase inhibitory activity of the methanolic extracts of selected Jordanian medicinal plants. Pharmacogn Mag. 2011 October; 7(28):320-4. doi: 10.4103/0973-1296.90413.
71. Lei Jiachuan, Yu Jianqing, Liao Zhixiong. Study on the antioxidant effect of cassia seed. “Chinese Journal of Information on Traditional Chinese Medicine” 2006, Issue 11.
72. Peng M J, Shi S Y, Chen L, Zhang S H, Cai P, Chen X Q. Online coupling solid-phase ligand-fishing with high-performance liquid chromatography-diode array detector-tandem mass spectrometry for rapid screening and identification of xanthine oxidase inhibitors in natural products. Anal Bioanal Chem. 2016 September; 408(24):6693-701.
73. Chien S C, Yang C W, Tseng Y H, Tsay H S, Kuo Y H, Wang S Y. Lonicera hypoglauca inhibits xanthine oxidase and reduces serum uric acid in mice. Planta Med. 2009 March; 75(4):302-6.
74. Nguyen M T, Awale S, Tezuka Y, Tran Q L, Watanabe H, Kadota S. Xanthine oxidase inhibitory activity of Vietnamese medicinal plants. Biol Pharm Bull. 2004 September; 27(9):1414-21.
75. Liu K, Wang W, Guo B H, Gao H, Liu Y, Liu X H, Yao H L, Cheng K. Chemical Evidence for Potent Xanthine Oxidase Inhibitory Activity of Ethyl Acetate Extract of Citrus aurantium L. Dried Immature Fruits. Molecules. 2016 Mar. 2; 21(3):302.
76. Hou P Y, Mi C, He Y, et al. Pallidifloside D from Smilax riparia enhanced allopurinol effects in hyperuricemia mice. Fitoterapia, vol. 105, no. 1, pp. 43-48, 2015.
77. Xu Tingting, Cheng Zhikai, Yin Lian, Xu Wenai. Study on the material basis of Smilax glabra in inhibiting the activity of xanthine oxidase. Chinese Medicinal Materials, Issue 4, 2012.
78. Wang Caiping. Research on the main components of mulberry branch extract and its mechanism of regulating organic ion transport and improving hyperuricemia. Nanjing University. 2009 Ph.D. Thesis. Supervisor, Kong Lingdong.
79. Hunyadi A, Liktor-Busa E, Márki A, Martins A, Jedlinszki N, Hsieh T J, Báthori M, Hohmann J, Zupkó I. Metabolic effects of mulberry leaves: exploring potential benefits in type 2 diabetes and hyperuricemia. Evid Based Complement Alternat Med. 2013; 2013:948627.
80. Sang M, Du G, Hao J, Wang L, Liu E, Zhang Y, Wang T, Gao X, Han L. Modeling and optimizing inhibitory activities of Nelumbinis folium extract on xanthine oxidase using response surface methodology. J Pharm Biomed Anal. 2017 May 30; 139:37-43.
81. Tu P T, Tawata S. Anti-Oxidant, Anti-Aging, and Anti-Melanogenic Properties of the Essential Oils from Two Varieties of Alpinia zerumbet. Molecules. 2015 Sep. 14; 20(9): 16723-40.
82. Li Li, Dai Lizhen, Yang Jing, Fang Jide. Screening of Xanthine Oxidase Inhibitors in Chinese Medicine Extracts. Journal of Wuhan Institute of Technology; Volume 32, Issue 3 (2010/03/30), P44-46.
83. Hong Zihuan, Lai Ruisheng, Lin Suyue, Yang Jie, Li Yalin. Danang ginger rhizome (galangal) inhibits the activity of xanthine oxidase. Taiwan Agricultural Research; Volume 66, Issue 4 (2017/12/31), P333-342.
84. Nguyen M T, Awale S, Tezuka Y, Ueda J Y, Tran Ql, Kadota S. Xanthine oxidase inhibitors from the flowers of Chrysanthemum sinense. Planta Med. 2006 January; 72(1):46-51.
85. Song H P, Zhang H, Fu Y, Mo H Y, Zhang M, Chen J, Li P. Screening for selective inhibitors of xanthine oxidase from Flos Chrysanthemum using ultrafiltration LC-MS combined with enzyme channel blocking. J Chromatogr B Analyt Technol Biomed Life Sci. 2014 Jun. 15; 961:56-61.
86. Honda S, Kawamoto S, Tanaka H, Kishida H, Kitagawa M, Nakai Y, Abe K, Hirata D. Administered chrysanthemum flower oil attenuates hyperuricemia: mechanism of action as revealed by DNA microarray analysis. Biosci Biotechnol Biochem. 2014; 78(4):655-61.
87. Lee Y S, Son E, Kim S H, Lee Y M, Kim O S, Kim D S. Synergistic Uric Acid-Lowering Effects of the Combination of <i>Chrysanthemum indicum</i>Linne Flower and <i>Cinnamomum cassia</i> (L.) J. Persl Bark Extracts. Evid Based Complement Alternat Med. 2017; 2017:9764843.
88. Wang Z, Kwon S H, Hwang S H, Kang Y H, Lee J Y, Lim S S. Competitive binding experiments can reduce the false positive results of affinity-based ultrafiltration-HPLC: A case study for identification of potent xanthine oxidase inhibitors from Perilla frutescens extract. J Chromatogr B Analyt Technol Biomed Life Sci. 2017 Mar. 24; 1048:30-37.
89. Huo L N, Wang W, Zhang C Y, Shi H B, Liu Y, Liu X H, Guo B H, Zhao D M, Gao H. Bioassay-Guided Isolation and Identification of Xanthine Oxidase Inhibitory Constituents from the Leaves of Perilla frutescens. Molecules. 2015 Sep. 25; 20(10):17848-59.
90. Yu Juan, Wang Xiaomei. The inhibitory effect of Coix Seed and Poria coix extracts on xanthine oxidase. Chinese Medicines and Clinics, 2014, Issue 1, pages 30-32.
91. Costantino L, Albasini A, Rastelli G, Benvenuti S. Activity of polyphenolic crude extracts as scavengers of superoxide radicals and inhibitors of xanthine oxidase. Planta Med. 1992 August; 58(4):342-4.
92. Liu F, Deng C, Cao W, Zeng G, Deng X, Zhou Y. Phytochemicals of Pogostemon Cablin (Blanco) Benth. aqueous extract: Their xanthine oxidase inhibitory activities. Biomed Pharmacother. 2017 May; 89:544-548.
93. Kim Yi. Will mandatory folic acid fortification prevent or promote cancer? Am J Clin Nutr 80: 1123-1128, 2004.
94. Choi S W and Mason J B: Folate status: Effects on pathways of colorectal carcinogenesis. J Nutr 132 (Suppl 8): S2413-S2418, 2002.
95. Varela-Moreiras G, Murphy M M, Scott J M. Cobalamin, folic acid, and homocysteine. Nutr Rev. 2009 May. 67 Suppl 1:S69-72.
96. Reynolds E H, Rothfeld P, Pincus J H. Neurological disease associated with folate deficiency. Br Med J. 1973 May 19; 2(5863):398-400.
97. Zaric B L, Obradovic M, Bajic V, Haidara M A, Jovanovic M, Isenovic E R. Homocysteine and Hyperhomocysteinaemia. Curr Med Chem. 2018 Mar. 12. doi: 10.2174/0929867325666180313105949. [Epub ahead of print]
98. Joshi R Adhikari SPatro BS et al. Free radical scavenging behavior of folic acid: evidence for possible antioxidant activity. Free Radic Biol Med. 2001; 30:1390-1399.
99. Hathcock J N. Vitamins and minerals: efficacy and safety. Am J Clin Nutr. 1997 August; 66(2):427-37.
100. Field M S, Stover P J. Safety of folic acid. Ann N Y Acad Sci. 2018 February; 1414(1):59-71.
101. Kim Yi. Will mandatory folic acid fortification prevent or promote cancer? Am J Clin Nutr 2004; 80(5): 1123-1128.
102. Wien T N, Pike E, Wisløff T, Staff A, Smeland S, Klemp M. Cancer risk with folic acid supplements: a systematic review and meta-analysis. BMJ Open. 2012 Jan. 12; 2(1):e000653.
103. Ebbing M, Bønaa KH, Nygård O, Arnesen E, Ueland P M, Nordrehaug J E, et al. Cancer incidence and mortality after treatment with folic acid and vitamin B12. JAMA 2009; 302(19): 2119-2126.

104. Kalckar H M, Klenow H. Milk xanthopterin oxidase and pteroylglutamic acid. J. Biol. Chem. 1948; 172,349-52.

105. Kalckar H M, Kjeldgaard N O, Klenow H. 2-Amino-4hydroxy-6-formylpteridine, an inhibitor of purine and pterine oxidases. Biochim Biophys Acta. 1950 June; 5(3/4):586-94.
106. Lewis A S, Murphy L, McCalla C, Fleary M, Purcell S. Inhibition of mammalian xanthine oxidase by folate compounds and amethopterin. J Biol Chem. 1984 Jan. 10; 259(1):12-5.
107. Spector T, Ferone R. Folic acid does not inactivate xanthine oxidase. J Biol Chem. 1984 Sep. 10; 259(17):10784-6.
108. Nishino T, Tsushima K. Interaction of milk xanthine oxidase with folic acid. Inhibition of milk xanthine oxidase by folic acid and separation of the enzyme into two fractions on Sepharose 4B/folate gel. J Biol Chem. 1986 Aug. 25; 261(24):11242-6.
109. Maciel M E, Castro G D, Castro J A. Inhibition of the rat breast cytosolic bioactivation of ethanol to acetaldehyde by some plant polyphenols and folic acid. Nutr Cancer. 2004; 49(1):94-9.
110. Verhaar M C, Wever R M, Kastelein J J, van Dam T, Koomans H A, Rabelink T J. 5-methyltetrahydrofolate, the active form of folic acid, restores endothelial function in familial hypercholesterolemia. Circulation. 1998 Jan. 27; 97(3):237-41.
111. Akhtar M J, Khan M A, Ahmad I. Photodegradation of folic acid in aqueous solution, J. Pharm. Biomed. Anal. 19 (1999) 269-275.
112. Branda R F, Eaton J W. Skin color and nutrient photolysis: an evolutionary hypothesis, Science 201 (1978) 625-626.
113. Hirakawa K, Suzuki H, Oikawa S, Kawanishi S. Sequencespecific DNA damage induced by ultraviolet A-irradiated folic acid via its photolysis product, Arch. Biochem. Biophys. 410 (2003) 261-268.
114. Jamil A M, Ataullah K M, Ahmad I. Identification of photoproducts of folic acid and its degradation pathways in aqueous solution, J. Pharm. Biomed. Anal. 31 (2003) 579-588.
115. Lucock M, Yates Z, Glanville T, Leeming R, Simpson N, Daskalakis I. A critical role for B-vitamin nutrition in human developmental and evolutionary biology, Nutrit. Res. 23 (2003) 1463-1475.
116. Thomas A H, Suarez G, Cabrerizo F M, Martino R, Capparelli A L. Study of the photolysis of folic acid and 6-formylpterin in acid aqueous solutions, Photochem. Photobiol. A: Chem. 135 (2000) 147-154.
117. Off M K, Steindal A E, Porojnicu A C, Juzeniene A, Vorobey A, Johnsson A, Moan J. Ultraviolet photodegradation of folic acid. J Photochem Photobiol B. 2005 Jul. 1; 80(1):47-55.
118. Keebaugh A C, Thomas J W. The Evolutionary Fate of the Genes Encoding the Purine Catabolic Enzymes in Hominoids, Birds, and Reptiles. Mol Biol Evol. 2010 June; 27(6): 1359-1369.
119. Keith C K, Broach W J, et al. Xanthine oxidase and tyrosinase in the livers of chicks receiving graded levels of dietary pteroylglutamic acid. J Biol Chem. 1948 December; 176(3):1095-1101.
120. Williams J N Jr, Nichol C A, Elvehjem C A. Relation of dietary folic acid and vitamin B12 to enzyme activity in the chick. J Biol Chem. 1949 September; 180(2):689-94.
121. Jing M, Munyaka P M, Tactacan G B, Rodriguez-Lecompte J C, O K, House J D. Performance, serum biochemical responses, and gene expression of intestinal folate transporters of young and older laying hens in response to dietary folic acid supplementation and challenge with Escherichia coli lipopolysaccharide. Poult Sci. 2014 January; 93(1):122-31.
122. Srivastava M, Chandra D, Kale R K. Modulation of radiation-induced changes in the xanthine oxidoreductase system in the livers of mice by its inhibitors. Radiat Res. 2002 March; 157(3):290-7.
123. Wu X, Liu J, Zhang J, et al. Folic acid reverses uric acid crystal-induced surface OAT1 internalization by inhibiting RhoA activity in uric acid nephropathy. Molecular Medicine Reports. 2016; 13(3):2385-2392.
124. Cui S, Li W, Lv X, Wang P, Gao Y, Huang G. Folic Acid Supplementation Delays Atherosclerotic Lesion Development by Modulating MCP1 and VEGF DNA Methylation Levels. In Vivo and In Vitro. Int J Mol Sci. 2017 May 5; 18(5).
125. Carnicer R, Navarro M A, Arbonés-Mainar J M, Acín S, Guzman M A, Surra J C, Arnal C, de Las Heras M, Blanco-Vaca F, Osada J. Folic acid supplementation delays atherosclerotic lesion development in apoE-deficient mice. Life Sci. 2007 Jan. 23; 80(7):638-43.
126. Qipshidze N, Tyagi N, Sen U, Givvimani S, Metreveli N, Lominadze D, Tyagi S C. Folic acid mitigated cardiac dysfunction by normalizing the levels of tissue inhibitor of metalloproteinase and homocysteine-metabolizing enzymes postmyocardial infarction in mice. Am J Physiol Heart Circ Physiol. 2010 November; 299(5):H1484-93.
127. Hwang S Y, Siow Y L, Au-Yeung K K, House J, O K. Folic acid supplementation inhibits NADPH oxidase-mediated superoxide anion production in the kidney. Am J Physiol Renal Physiol. 2011 January; 300(1):F189-98.
128. Lu R, Wang X, Sun D F, Tian X Q, Zhao S L, Chen Y X, Fang J Y. Folic acid and sodium butyrate prevent tumorigenesis in a mouse model of colorectal cancer. Epigenetics. 2008 November; 3(6):330-5.
129. Ishiguro L, Yang M, Sohn K J, Streutker C J, Grin A, Croxford R, Kim Y I., Folic Acid Supplementation Adversely Affects Chemosensitivity of Colon Cancer Cells to 5-fluorouracil. Nutr Cancer. 2016 July; 68(5):780-90.
130. Lyu L C, Hsu C Y, Yeh C Y, Lee M S, Huang S H, Chen C L. A case-control study of the association of diet and obesity with gout in Taiwan. Am J Clin Nutr 2003; 78:690-701.
131. Xu X, Qin X, Li Y, Sun D, Wang J, Liang M, Wang B, Huo Y, Hou F F. Efficacy of Folic Acid Therapy on the Progression of Chronic Kidney Disease: The Renal Substudy of the China Stroke Primary Prevention Trial. JAMA Intern Med. 2016 Oct. 1; 176(10):1443-1450.
132. Lu J M, Yao Q, Chen C. 3,4-Dihydroxy-5-nitrobenzaldehyde (DHNB) is a potent inhibitor of xanthine oxidase: a potential therapeutic agent for treatment of hyperuricemia and gout. Biochem Pharmacol. 2013 Nov. 1; 86(9):1328-37.
133. Wu X, Wakamiya M, Vaishnav S, Geske R, Montgomery C Jr, Jones P, Bradley A, Caskey C T. Hyperuricemia and urate nephropathy in urate oxidase-deficient mice. Proc Natl Acad Sci USA. 1994 Jan. 18; 91(2):742-6.

### Example 4

### Development of Health Food Supplements/Antioxidants for Controlling Hyperuricemia and Oxidative Stress

As described herein, the inventors determined the effect of xanthine oxidase (XO) inhibition for 112 standard herbal extracts obtained from Bulk Supplements.com (111) and Trustworthy Herbs Inc (1) by an in vitro enzyme activity assay. All extracts in a DSMO-soluble fraction at a final concentration of 166.7 ug/ml except for a few extracts at lower concentrations due to a solubility issue was used in the assay. There are 15 herb extracts that showed a strong effect of XO inhibition (>40%). There are 19 herb extracts, which have an XO inhibition rate between 20% to 40%. The rest of 78 herb extracts had a weak effect of XO inhibition (<20%). 5 out of 15 extracts (>40% XO inhibition) are not previously reported; 7 out of 19 extracts (20-40% OXI) are not previously reported. In addition, a dose dependent XO inhibition assay was performed for each of these 15 extracts and 6 extracts, which showed a 20-40% XO inhibitory effect at a single dose (166.7 ug/ml). Concentration range of each extract from 0 to 250, or 333 ug/ml (8 to 10 doses was studied). All 21 extracts showed a good dose-dependent XO inhibition curve. IC₅₀ is an operational parameter defined as the concentration of inhibitor required for achieving 50% inhibition of the enzyme. The smaller IC₅₀, the more potent the inhibitor is. IC₅₀ for each extract is calculated. Top ten potent XO inhibitors are Milk Thistle (IC₅₀ 3.3 ug/ml), Japanese Thistle extract (IC₅₀ 3.8 ug/ml), Amla Fruit (IC₅₀ 5.6 ug/ml), Grape Seed (IC₅₀ 8 ug/ml), Pomegranate (40% Ellagic Acid, IC₅₀ 10 ug/ml), Green Tea (50% EGCG, IC₅₀ 11.5 ug/ml), Pine Bark (IC₅₀ 12 ug/ml), St John's Wort (IC₅₀ 14 ug/ml), African Mango seed (IC₅₀ 35 ug/ml), and Butcher's Broom (IC₅₀ 82 ug/ml). Furthermore, a relatively small concentration was selected of each of 8 herbal extracts and folic acid for a combination assay. Folic acid (0.167 uM) and extract at a defined concentration (ug/ml) were tested. Three out of 8 extracts (Grape Seed, Milk Thistle and Japanese Thistle extract) showed a synergistic XO inhibitory effect with folic acid; 3 out of 8 extracts (Amla Fruit, Green Tea and Pine Bark) showed an additive XO inhibitory effect with folic acid.

The inventors designed a dietary health supplement recipe, which contains five herbal extracts (Japanese thistle extract, Grape seeds extract, Amla extract, Pine bark extract, Chinese mint extract) and folic acid at a clinically relevant dose for animal testing. Allopurinol was used a positive control. Each mouse was I.P. injected with allatoxanamide, and then the mice were then oral gavaged with allopurinol or extract recipe, respectively. Blood was taken from facial vein at 1.5 hours and 3 hours after the treatment. Serum uric acid level was measured. Uricase inhibitor Allantoxanmide treatment (I.P. injection) significantly increased serum uric acid levels at 1.5 hours and 3 hours. Allopurinol at a clinically relevant dose significantly reduced serum uric acid levels in Allantoxanmide-treated mice at both 1.5 hours and 3 hours. Although clinically relevant dose of dietary health supplement recipe was less potent than Allopurinol, it also significantly reduced serum uric acid levels in Allantoxanmide-treated mice at both 1.5 hours and 3 hours. This effect showed a dose-dependent manner.

1. Data of 112 Standard Herb Medicine Extracts

Standard herbal extracts (111) were obtained from Bulk Supplements.com and 1 herbal extract obtained from Trustworthy Herbs Inc. Bulk Supplements Inc is located in Henderson, Nevada. Per the company's website, 1) it is a FDA-registered cGMP manufacturing and distribution facility; 2) the company is dedicated to maintaining all health code and government regulations, and 3) each supplement is tested in their in-house laboratory before distribution, ensuring that all products are safe for consumption. The standard herbal extracts (100 g) were in powder form and were packaged in a seal in the alumina bag. Daily serving dose of each extract is provided on the bag.

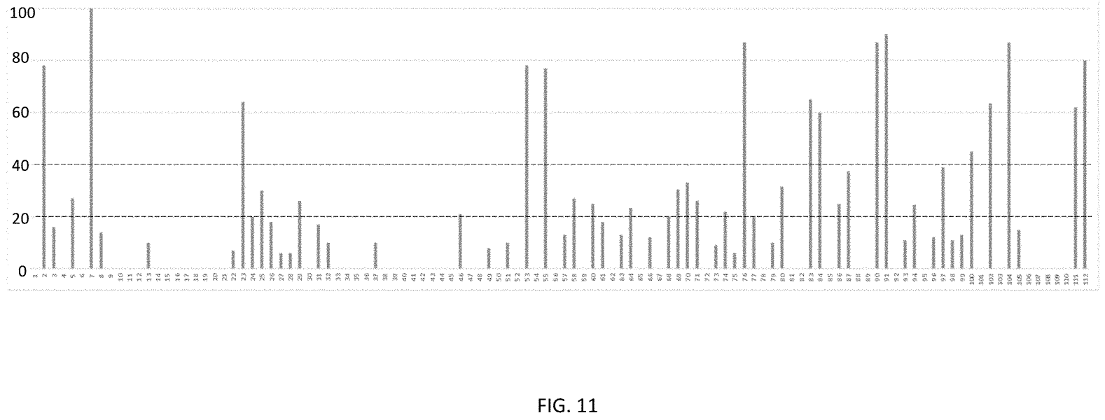

All extracts were in a DSMO-soluble fraction at a final concentration of 166.7 ug/ml except for a few extracts at lower concentrations because of solubility issues; these were mixed with 20 nM XO. The initial rate of uric acid formation in the reaction system was recorded; and XO inhibition rate of each herb extract was calculated. All data are shown in Table 9 and FIG. 11 (and the numbering on the x-axis in FIG. 11 corresponds to the order of the list of compounds in Table 9). There are 15 herb extracts that showed a strong effect of XO inhibition (>40%). There are 19 herb extracts, which have an XO inhibition rate between 20% to 40%. The rest of 78 herb extracts had a weak effect of XO inhibition (<20%).

The inventors performed a preliminary literature search (herb name, xanthine oxidase, English and Chinese) in both the Medline and Google scholar search engines, and it was found that:
5 out of 15 extracts (>40% XO inhibition) are not previously reported: Butcher's Broom Extract (64% XOI), Oolong Tea (60% XOI), Senna Leaf (45% XOI), Yohimbe bark (62% XOI) and Japanese Thistle extract (80% XOI at 33.3 ug/ml). XO inhibitory effects of other 10 extracts are previously reported: African Mango seed (78% XOI) (1,2), Amla Fruit (100% XOI) (5), Grape Seed (78% XOI) (7,8,9), Green Tea (50% EGCG, 77% XOI (10,11,12), Milk Thistle (87% XOI at 33.3 ug/ml) (19), Olive Leaf (65%) (21,22), Pine Bark (87% XOI) (25), Pomegranate (40% Ellagic Acid, 90% XOI) (26,27,28), Spearmint (63.6%) (15, 32) and St John's Wort (87% XOI) (33).
7 out of 19 extracts (20-40% OXI) are not previously reported: Butterbur (20% XOI), Caralluma bark (30% XOI), Echinacea root (21% XOI), Horse Chestnut fruit (23.5% XOI), Kola Nut (20% XOI), Magnolia Bark (22% XOI) and Muira Puama Bark (31.6% XOI).

112 items (111 from Bulk Supplements.com and 1 from Trustworthy Herbs Inc): single dose (166.7 ug/ml, DMSO-fraction):

15 extracts: >40% XO inhibition (5 out of 15 are not previously reported per our preliminary keyword search)

19 extracts: 20-40% XO inhibition (7 out of 19 are not previously reported per our preliminary keyword search)

78 extracts: <20% XO inhibition

TABLE 9
XO inhibitory effects of 112 herbal extracts (111 from Bulk
Supplements.com and 1 from Trustworthy Herbs Inc

| | | XOI % | |
|---|---|---|---|
| | | (166.7 | Previous |
| Herb extract and Description | Code | ug/ml) | report |
| Acai Berry Extract 4:1 Powder | ACAI100 | 0 | |
| #1, African Mango | AFMAN100 | 78 | Ref 1, 2 |
| seed Extract Powder | | | |
| Ajuga Turkest Extract Powder | AJTU100 | 16 | |
| Alfalfa Extract Powder | ALFA100 | 0 | |
| Aloe Vera Leaf Extract Powder | ALOE100 | 27 | Ref 3, 4 |
| American Ginseng | AMGINEX100 | 0 | |
| Extract Powder | | | |
| #2, Amla Fruit Powder | AMLA100 | 100 | Ref 5. |
| Andrographis Extract Powder | ANPA100 | 14 | |
| Aronia Extract Powder | ARON100 | 0 | |
| Artichoke Extract Powder | ART100 | 0 | |
| Astragalus Extract Powder | ASTR100 | 0 | |
| Bamboo Extract Powder | BAMB100 | 0 | |
| Bearberry Leaf Extract | BEAR100 | 10 | |
| (Uva Ursi) | | | |
| Bilberry Extract Powder | BIL100 | 0 | |
| Bitter Melon Extract Powder | MELON100 | 0 | |
| Black Cohosh Powder | BLKC100 | 0 | |
| Black Garlic Extract Powder | BKGRLC250 | 0 | |
| Blood Orange Extract Powder | BLDOR100 | 0 | |
| Boswellia Serrata | BOSW100 | 0 | |
| Extract Powder | | | |
| Broccoli Extract Powder | BROC100 | 0 | |
| Buckthorn Bark Extract Powder | BTHRN100 | 0 | |
| Burdock Extract Powder | BURD100 | 7 | |
| #3, Butcher's Broom | BROOM100 | 64 | NA |
| Extract Powder | | | |
| Butterbur Extract Powder | BTRBR25 | 20 | NA |
| Caralluma bark (wild) | CARFIM100 | 30 | NA |
| Extract Powder | | | |
| Cascara Sagrada Extract Powder | CASSA100 | 18 | |
| Cassia Seed Extract Powder | CASSS100 | 6 | |
| Cat's Claw Extract Powder | CTCLW100 | 6 | |
| Catuaba bark Bark | CAT100 | 26 | Ref 6. |
| Extract Powder | | | |
| Cayenne Extract Powder | CAYN100 | 0 | |
| Celery Seed Extract 4:1 Powder | CLYSD100 | 17 | |
| Chasteberry Extract | CBE-100 | 10 | |
| Chitosan Extract Powder | CHLOR100 | 0 | |
| Chlorella Blue-Green Algae | CHLOR100 | 0 | |
| Cinnamon Bark Powder Extract | CBE100 | 0 | |
| Cissus Quadrangularis Powder | CISSQ100 | 0 | |
| Cocoa Extract | COCOA100 | 10 | |
| Cordyceps Powder | CORDY100 | 0 | |
| Coriolus Versicolor | COVER100 | 0 | |
| (Turkey Tail) Powder | | | |
| Corydalis Extract Powder | CRYDLS100 | 0 | |
| Creek Yellow Grass Powder | CYG100 | | |
| Curcumin 95% Natural | CURC25 | 0 | |
| Turmeric Extract | | | |
| Dandelion Root Extract Powder | DAND100 | 0 | |
| Devil's Claw Extract Powder | DEVCLW100 | 0 | |
| Dragon Fruit Extract Powder | DGNFRT100 | 0 | |
| Echinacea root Extract Powder | EYEB100 | 21 | NA |
| Eyebright Extract Powder | EYEB100 | 0 | |
| Flaxseed Extract Powder | FLXPDR100 | 0 | |
| Fo-Ti Extract Powder | FOTI100 | 8 | |
| Garlic Extract | GARLIC100 | 0 | |
| Ginkgo Biloba Leaf Extract | GINK100 | 10 | |
| Ginseng Root Extract Powder | GINEX50 | 0 | |
| #4, Grape Seed Extract | GSE100 | 78 | Ref 7, 8, 9 |
| Grapefruit Seed | GFS100 | 0 | |
| Extract-100 g Pure Powder | | | |
| #5, Green Tea Pure | GTEGCG100 | 77 | Ref 10, |
| Extract 50% EGCG | | | 11, 12 |
| Guanabana Extract Powder | GNABNA100 | 0 | |
| Gymnema leaf Extract Powder | GYMN100 | 13 | |
| Gynostemma Extract Powder | GYST100 | 27 | Ref 13, 14 |
| Hawthorn Berry Extract Powder | HWTHNB100 | 0 | |
| Hawthorn Leaf Extract Powder | HWTHNLF100 | 25 | Ref 15 |
| Hibiscus Flower Extract Powder | HIBSC100 | 18 | |
| Hoodia Extract | HOOD100 | 0 | |
| Hops Extract Powder | HOP100 | 13 | |
| Horse Chestnut fruit | HORCHE100 | 23.5 | NA |
| Extract Powder | | | |
| Horsetail Extract Powder | HORS100 | 0 | |
| Kava Kava Extract Powder | KVA100 | 12 | |
| Kelp Extract Powder | KLP100 | 0 | |
| Kola Nut Extract Powder | KOLA100 | 20 | NA |
| Kudzu Root Extract Powder | KDZ100 | 30.3 | Ref 15, 16 |
| Lemon Balm Extract Powder | LMBLM100 | 33 | Ref 17 |
| Licorice Root Extract Powder | LCRT100 | 26 | Ref 18 |
| LongJack Extract | LJ25 | 0 | |
| Maca Root Extract | MACA100 | 9 | |
| Magnolia Bark Extract Powder | MAGN0100 | 22 | NA |
| Marshmallow Root | MARSH-100 | 6 | |
| Extract Powder | | | |
| #6, Milk Thistle Extract Powder | MTE100 | 87% | Ref 19 |
| | | (33.3 | |
| | | ug/ml) | |
| Moringa Extract Powder | MORLE100 | 20 | Ref 20 |
| Motherwort Extract Powder | MOTH100 | 0 | |
| Mucuna Pruriens Extract Powder | MUPR100 | 10 | |
| Muira Puama Bark | MUPU100 | 31.6 | NA |
| Extract Powder | | | |
| Nettle Extract Powder- | NTLLF100 | 0 | |
| Oat Straw Extract Powder | OATSTW100 | 0 | |
| #7, Olive Leaf Extract Powder | OLE100 | 65 | Ref 21, 22 |
| #8, Oolong Tea (leaf) | OOLT100 | 60 | NA |
| Extract Powder | | | |
| Spirulina (Organic) Powder | SPIORG100 | 0 | |
| Papaya Fruit Extract Powder | PAPFRUIT100 | 0-25 | Ref 23, 24 |
| Papaya Seed Powder | PAPSEED100 | 37.5 | Ref 23 |
| Parsley Extract Powder | PARS100 | 0 | |
| Passion Flower Extract Powder | PASSFLO100 | 0 | |
| #9, Pine Bark Extract | PBE100 | 87 | Ref 25 |
| #10, Pomegranate | POM40100 | 90 | Ref 26, |
| Extract (40% Ellagic | | | 27, 28 |
| Acid) | | | |
| Portulaca Oleracea | POR100 | 0 | |
| (Purslane) Extract | | | |
| Pumpkin Seed Extract Powder | PUMP100 | 11 | |
| Red Clover (steam-leaf) | RCE100 | 24.5 | Ref 29 |
| Extract Powder | | | |
| Red Yeast Rice Extract (RYR) | RYR100 | 0 | |
| Reishi Mushroom Powder | REI100 | 12 | |
| Rosemary Extract Powder | RSMRY100 | 39 | Ref 30, 31 |
| Sage Extract Powder | SAGE100 | 11 | |
| Sarsaparilla Root Powder | SARS100 | 13 | |
| #11, Senna Leaf Extract | SENN250 | 45 | NA |
| Slippery Elm Bark | SLPEM100 | 0 | |
| Extract Powder | | | |
| #12, Spearmint Leaf | SPMNT100 | 63.6 | Ref 15, 32 |
| Extract Powder | | | |
| Spinach Extract Powder | SPIN100 | 0 | |
| #13, St John's Wort Extract | SJW10 | 87 | Ref 33 |
| Taxillus Chinensis | TAXCHI100 | 15 | |
| Danser Extract Powder | | | |
| Turkey Rhubarb Extract Powder | RHU100 | 0 | |
| Valerian Root Extract | VRE-100 | 0 | |
| White Mulberry Fruit | WHML100 | 0 | |
| Extract Powder | | | |
| White Willow Bark Extract | WVVB | 100 0 | |
| Wild Yam Extract Powder | YAM100 | 0 | |
| #14, Yohimbe bark | YMB500 | 62 | NA |
| Extract Powder | | | |
| #15, Extract 5:1 | EXD003 | 80% | NA |
| 100 g \| Japanese Thistle | | at 33.3 | |
| Extract 5:1 | | ug/ml | |

NA means: the extract was not previously reported for XO inhibition
Blank means: the extract has no XO inhibition as tested, and no literature search was performed

2. Data of Dose-Dependent of 21 Standard Herb Medicine Extracts

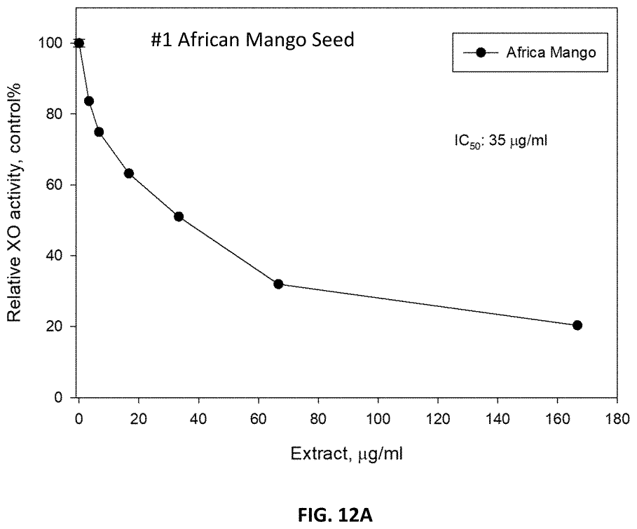

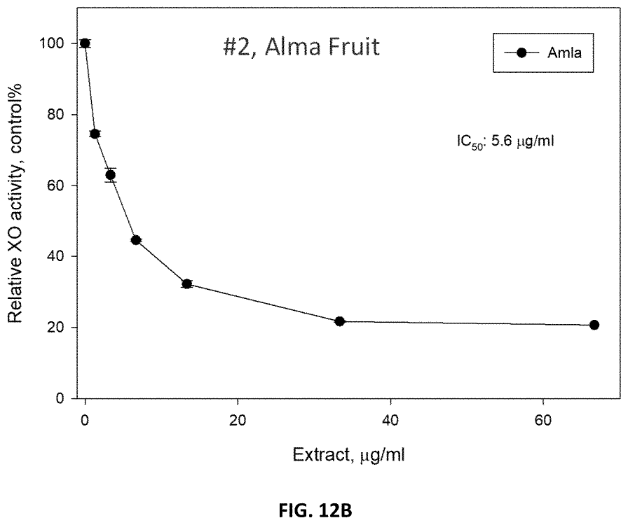

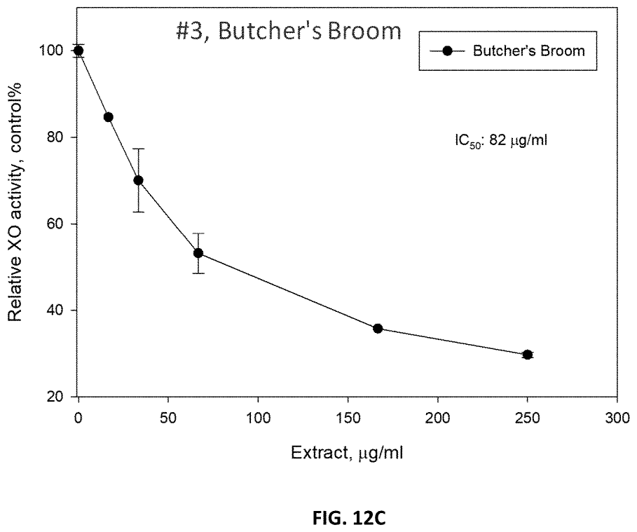

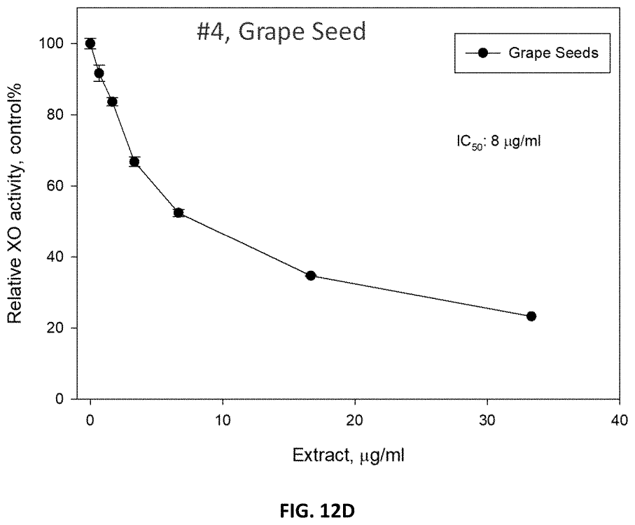

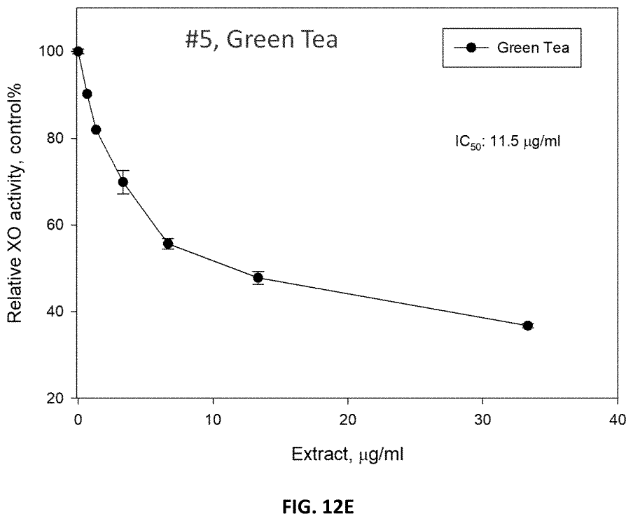

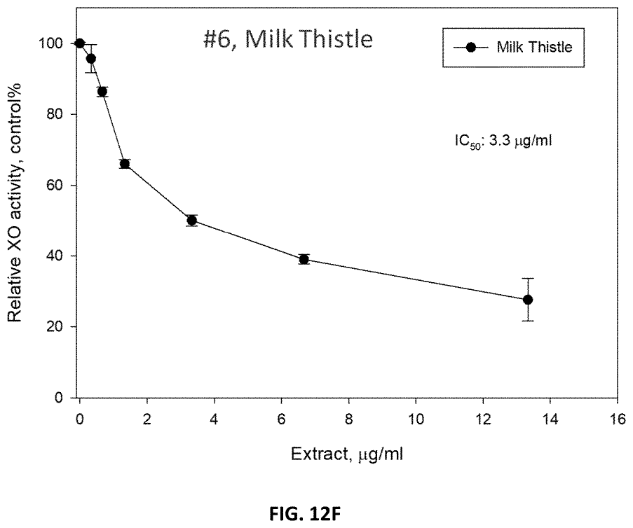

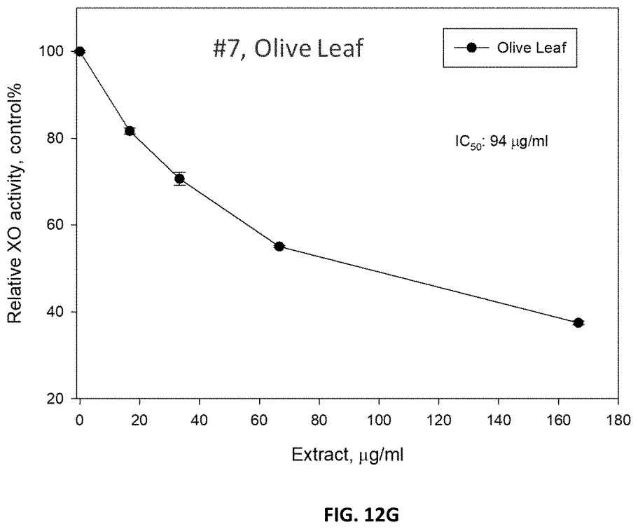

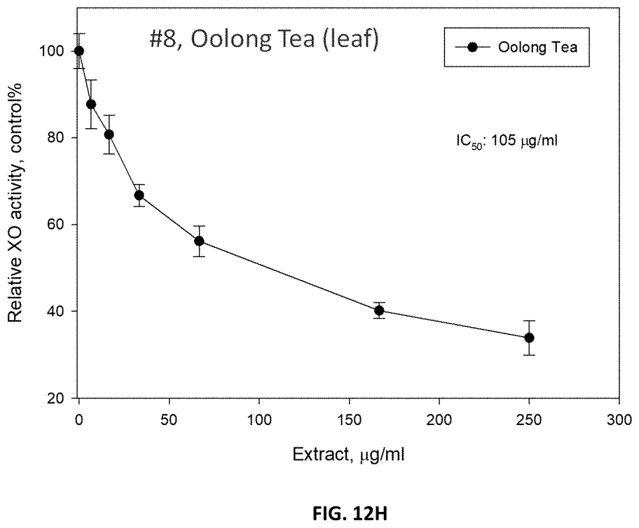

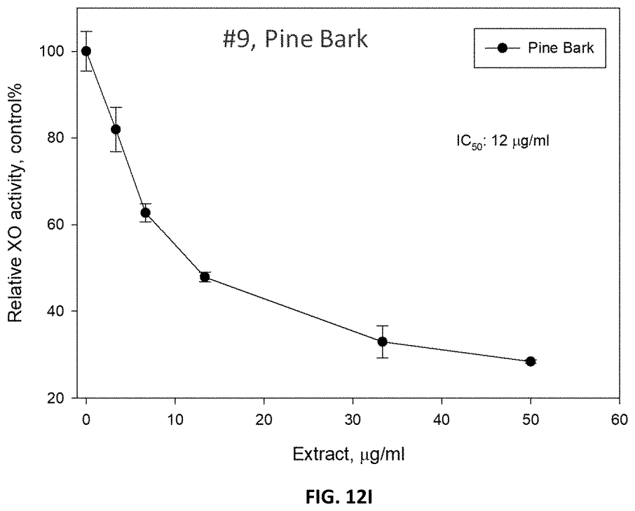

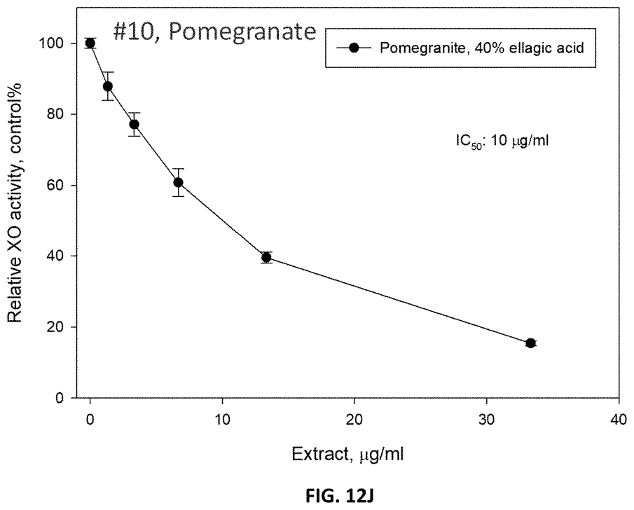

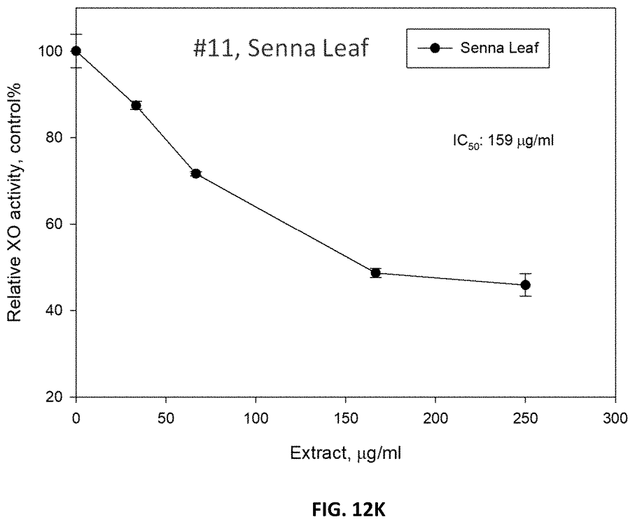

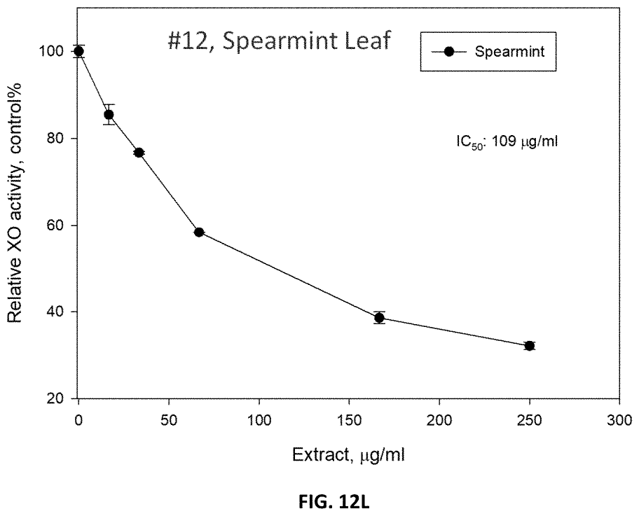

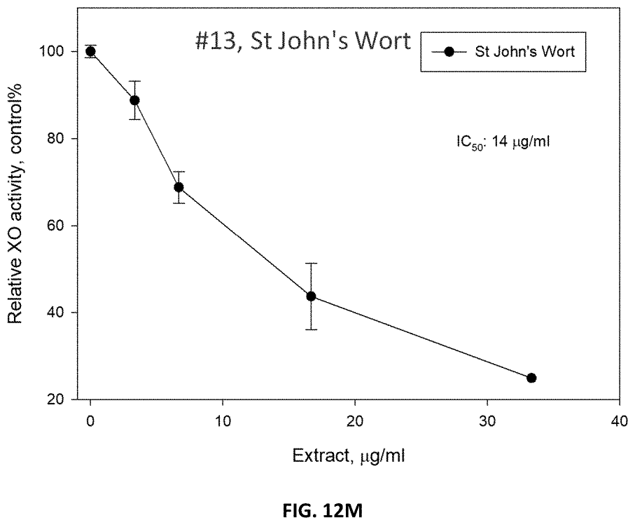

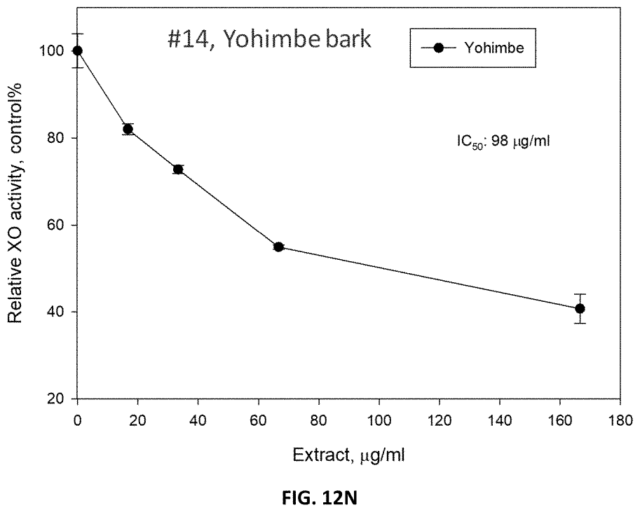

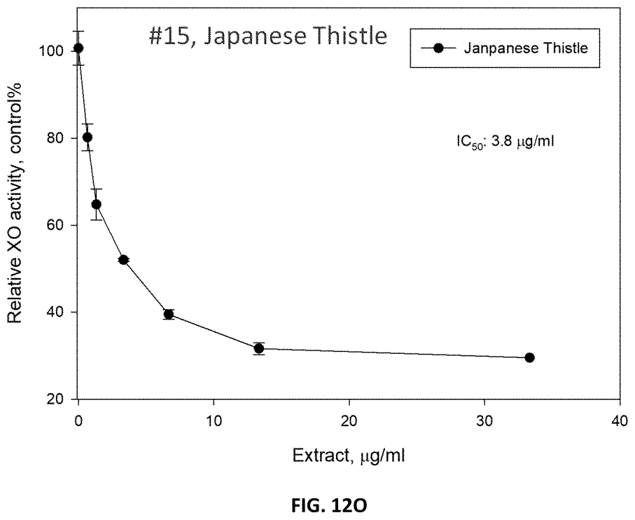

From initial screen of XO inhibitory effects of 112 herbal extracts with single dose (166.7 ug/ml), 15 extracts had more than 40% inhibition of XO activity. Furthermore, a dose dependent XO inhibition assay was performed for each of these 15 extracts. In addition, the inventors also tested a dose-dependent study for 6 extracts, which showed a 20-40% XO inhibitory effect at a single dose (166.7 ug/ml). Concentration range of each extract from 0 to 250, or 333 ug/ml (8 to 10 doses was studied). All 21 extracts showed a nice dose-dependent XO inhibition curve. IC₅₀ is an operational parameter defined as the concentration of inhibitor required for achieving 50% inhibition of the enzyme. The smaller IC₅₀, the more potent the inhibitor is. IC₅₀ for each extract is calculated (Table 10, FIG. 12). Top ten potent XO inhibitors are Milk Thistle (IC₅₀ 3.3 ug/ml), Japanese Thistle extract (IC₅₀ 3.8 ug/ml), Amla Fruit (IC₅₀ 5.6 ug/ml), Grape Seed (IC₅₀ 8 ug/ml), Pomegranate (40% Ellagic Acid, IC₅₀ 10 ug/ml), Green Tea (50% EGCG, IC₅₀ 11.5 ug/ml), Pine Bark (IC₅₀ 12 ug/ml), St John's Wort (IC₅₀ 14 ug/ml), African Mango seed (IC₅₀ 35 ug/ml), and Butcher's Broom (IC₅₀ 82 ug/ml). These data is useful for understanding the potency of these extracts and provides rational design for combination assay of each extract with folic acid and 5-MTHF or multiple extracts with folic acid and 5-MTHF.

TABLE 10
IC₅₀ (XO inhibitory effect) of 21 herbal extracts

| | | XOI % | | |
|---|---|---|---|---|
| Herb extract and | | (166.7 | IC₅₀ | Previous |
| Description | Code | ug/ml) | (ug/ml) | report |
| #1, African Mango | AFMAN100 | 78 | 35 | Ref 1, 2 |
| seed Extract | | | | |
| Powder | | | | |
| #2, Amia Fruit | AMLA100 | 100 | 5.6 | Ref 5 |
| Powder | | | | |
| #3, Butcher's | BROOM100 | 64 | 82 | NA |
| Broom Extract | | | | |
| Powder | | | | |
| #4, Grape Seed | GSE100 | 78 | 8 | Ref 7, 8, 9 |
| Extract | | | | |
| #5, Green Tea Pure | GTEGCG100 | 77 | 11.5 | Ref 10, |
| Extract 50% | | | | 11, 12 |
| EGCG | | | | |
| #6, Milk Thistle | MTE100 | 87% | 3.3 | Ref 19 |
| Extract Powder | | (33.3 ug/ml) | | |
| #7, Olive Leaf | OLE100 | 65 | 94 | Ref 21, 22 |
| Extract Powder | | | | |
| #8, Oolong Tea | OOLT100 | 60 | 105 | NA |
| (leaf) Extract | | | | |
| Powder | | | | |
| #9, Pine Bark | PBE100 | 87 | 12 | Ref 25 |
| Extract | | | | |
| #10, Pomegranate | POM40100 | 90 | 10 | Ref 26, |
| Extract (40% | | | | 27, 28 |
| Ellagic Acid) | | | | |
| #11, Senna Leaf | SENN250 | 45 | 159 | NA |
| Extract | | | | |
| #12, Spearmint | SPMNT100 | 63.6 | 109 | Ref 15, 32 |
| Leaf Extract | | | | |
| Powder | | | | |
| #13, St John's | SJW10 | 87 | 14 | Ref 33 |
| Wort Extract | | | | |
| #14, Yohimbe | YMB500 | 62 | 98 | NA |
| barkExtract | | | | |
| Powder | | | | |
| #15, Extract 5:1 | EXD003 | 80% at 33.3 | 3.8 | NA |
| 100 g \| Japanese | | ug/ml | | |
| Thistle | | | | |
| Caralluma bark | CARFIM100 | 30 | >170 | NA |
| (wild) Extract | | | | |
| Powder | | | | |
| Kudzu Root | KDZ100 | 30.3 | >170 | Ref 15, 16 |
| Extract Powder | | | | |
| Lemon Balm | LMBLM100 | 33 | >250 | Ref 17 |
| Extract Powder | | | | |
| Muira Puama | MUPU100 | 31.6 | >250 | NA |
| Bark Extract | | | | |
| Powder | | | | |
| Papaya Seed | PAPSEED100 | 37.5 | 200 | Ref 23 |
| Powder | | | | |
| Rosemary | RSMRY100 | 39 | 280 | Ref 30, 31 |
| Extract Powder | | | | |

NA means: the extract was not previously reported for XO inhibition

3. Data of Combination of Single Herb Medicine Extract with Folic Acid

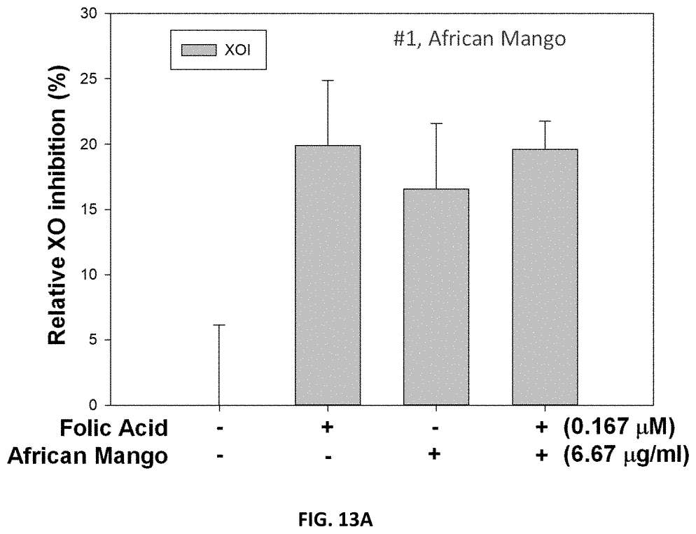

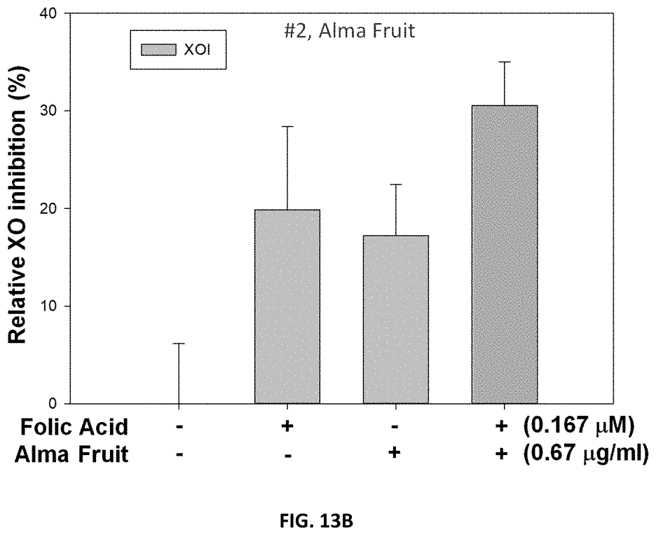

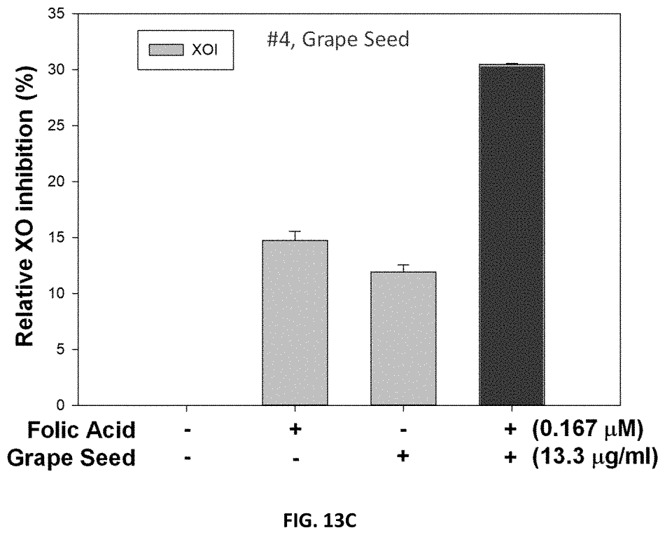

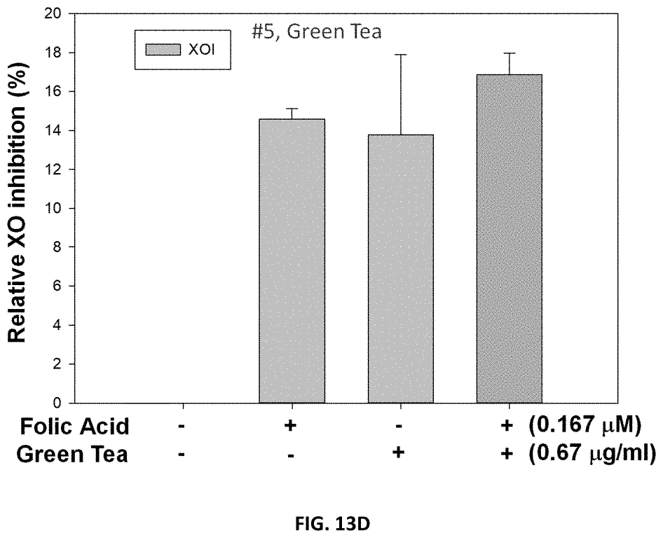

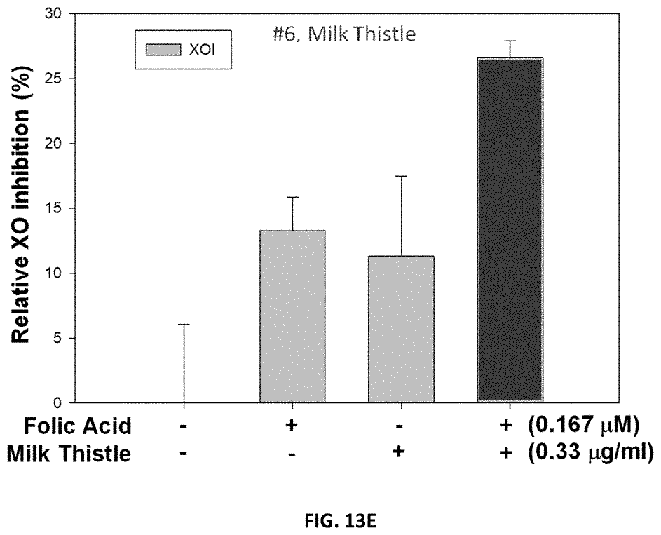

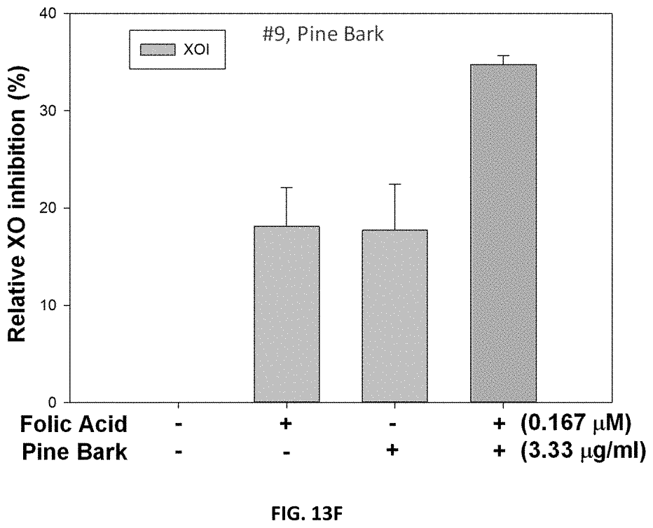

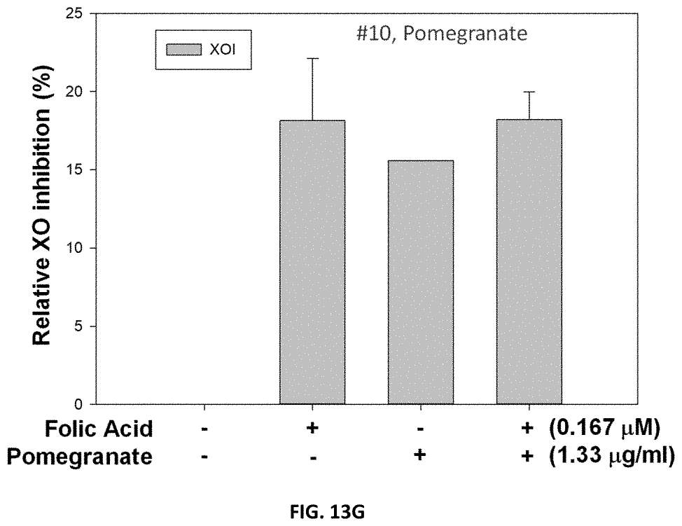

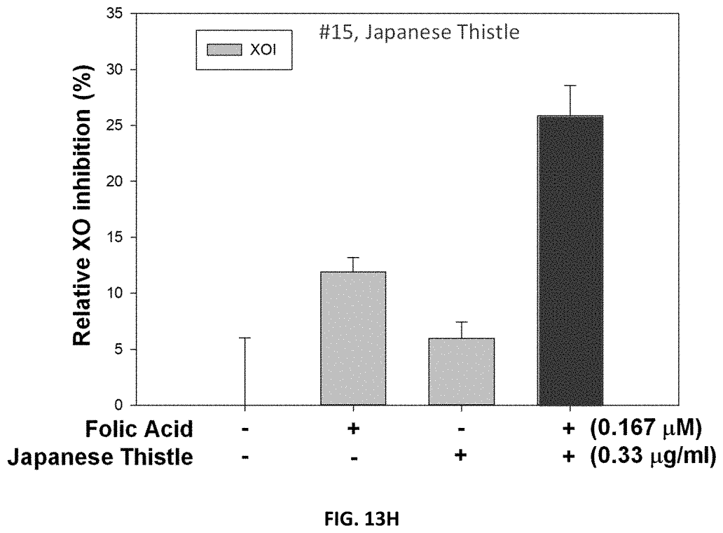

Based on the result of dose-dependent XO inhibition experiments for herb extracts and folic acid, a relatively small concentration of each of 8 herbal extracts and folic acid were selected for a combination assay. One of the important advantages of such combination supplements is enhancement of XO inhibitory function of the supplement; while reducing the dose of each component, thereby reducing potential side effects of the supplement for long-term use. Folic acid (0.167 uM) and extract at a defined concentration (ug/ml) were separately added into the XO reaction system without pre-mixing. XO activity was measured (FIG. 13). Three out of 8 extracts (Grape Seed, Milk Thistle and Japanese Thistle extract) showed a synergistic XO inhibitory effect with folic acid; 3 out of 8 extracts (Amla Fruit, Green Tea and Pine Bark) showed an additive XO inhibitory effect with folic acid; and 2 out of 8 extracts (African Mango and Pomegranate) did not shown any additive or synergistic XO inhibitory effect with folic acid.

4. Data of the Hypouricemic Effect of One Dietary Health Supplement Recipe in Allantoxanamide-Treated Mice

Design dietary health supplement recipe (combination). One of the important advantages of combination of several herbal extracts and folic acid is enhancement of XO inhibitory function of the supplement; while reducing the dose of each component, thereby reducing potential side effects of the supplement for long-term use. Selected herbal extracts should have a strong XO inhibitory effect in vitro, and show an additive or synergistic effect with folic acid. Herbal extracts, which are not reported for their XO inhibitory function, are utilized, in specific embodiments. Based on this rationale, the inventors have designed one exemplary recipe, which contains five herbal extracts and folic acid (Table 11).

TABLE 11
Design of an example of a dietary health supplement
recipe (combination)

| | | | Human (60 kg) | + Folic | Report |
|---|---|---|---|---|---|
| Component | XOI IC₅₀ | | Daily Dose | Acid | XOI |
| Japanese Thistle | 3.8 | ug/ml | 250 mg (4.2 mg/kg) | Synergistic | No |
| Extract | | | | | |
| Grape Seed | 8 | ug/ml | 200 mg (3.3 mg/kg) | Synergistic | Yes |
| Extract | | | | | |
| Amla Fruit | 5.6 | ug/ml | 800 mg (13.3 | additive | Yes |
| Extract | | | mg/kg) | | |
| Pine Bark | 12 | ug/ml | 250 mg (4.2 mg/kg) | additive | Yes |
| Extract | | | | | |
| Chinese Mint | 34 | ug/ml | 1000 mg (16.7 | additive | No |
| Extract | | | mg/kg) | | |
| Folic acid | 0.75 | uM | 800 ug (13.3 ug/kg) | | Yes |
| | | | Mouse dose | | |
| | | | Low dose: 41.7 | | |
| | | | mg/kg | | |
| | | | High dose: 83.4 | | |
| | | | mg/kg | | |

Mouse experiment design, method and results. The extract combination samples were prepared by weighing 4.2 mg Japanese thistle extract, 3.3 mg Grape seeds extract, 13.3 mg Amla extract, 4.2 mg Pine bark extract, and 16.7 mg Chinese mint extract; mixing them with 10 ml 1% PEG400, and making a stock solution of 41.7 mg/10 ml. Folic acid solution was prepared separately by dissolving 2.6 mg folic acid in 1 ml 0.1 m potassium phosphate buffer, then 6 ul of this FA solution was added to the 10 ml extract solution. As a positive control, Allopurinol, a clinical XO-inhibitor drug, was used at 13.3 mg/kg for the mouse experiment. Human dose of Allopurinol is about 800 mg daily.

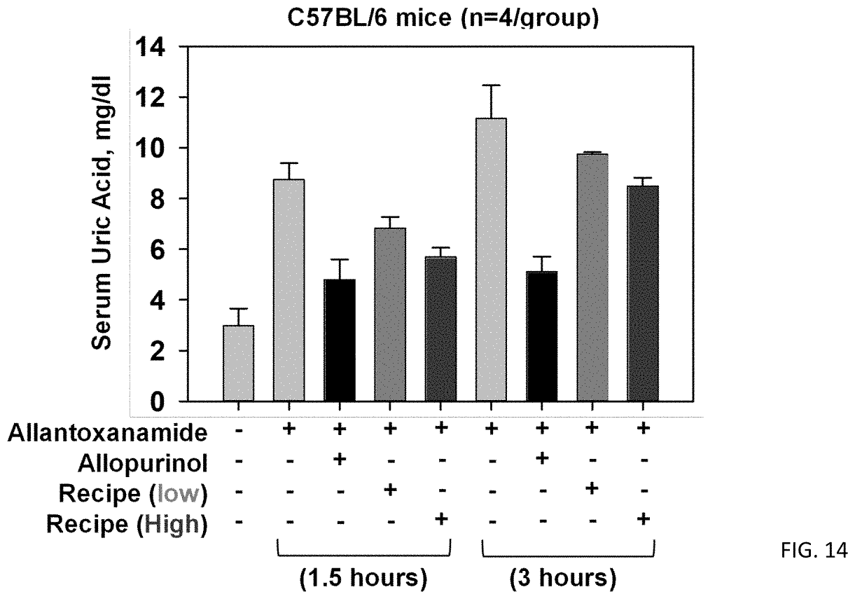

Adult C57BL/6 mice (about 20 g body weight) were used. Four groups of mice (n=4 per group) were assigned for different treatments or controls (Table 12). Intraperitoneal (i.p.) injection of uricase inhibitor allantoxanamide can effectively block the conversion of uric acid to 5-hydroxyisourate and thus cause a marked increase in serum uric acid levels in mice, providing a hyperuricemic animal model (34). Each mouse was ip injected with allatoxanamide at a dose of 200 mg/kg, the mice were then oral gavaged with 200 ul to 250 ul (for 20 to 25 g mice) allopurinol or extract solution, respectively. Blood was taken from facial vein at 1.5 hours and 3 hours after the treatment. Serum uric acid level was measured with a phosphotunstate method. Uricase inhibitor Allantoxanmide treatment (I.P. injection) significantly increased serum uric acid levels at 1.5 hours and 3 hours; these data are consistent with those in a previous publication (34). As a positive control, Allopurinol at a clinically relevant dose significantly reduced serum uric acid levels in Allantoxanmide-treated mice at both 1.5 hours and 3 hours. Although clinically relevant dose of dietary health supplement recipe was less potent than Allopurinol, it also significantly reduced serum uric acid levels in Allantoxanmide-treated mice at both 1.5 hours and 3 hours. This effect showed a dose-dependent manner (FIG. 14).

TABLE 12
Mouse assignments for studying the hypouricemic
effect of dietary health supplement

| Group | Allantoxanamide | | |
|---|---|---|---|
| (n = 4) | (200 mg/kg, IP) | Treatment | End Point |
| 1 | − | − | Serum uric acid (1.5 and 3 |
| | | | hours) |
| 2 | + | Allopurinol | Serum uric acid (1.5 and 3 |
| | | (13.3 mg/kg) | hours) |
| 3 | + | Recipe low dose | Serum uric acid (1.5 and 3 |
| | | (41.7 mg/kg) | hours) |
| 4 | + | Recipe high dose | Serum uric acid (1.5 and 3 |
| | | (83.4 mg/kg) | hours) |

### REFERENCES FOR EXAMPLE 4

1. Huang Ping. Study on the structural modification of mangiferin and its aglycon and the inhibitory activity of xanthine oxidase, synthesis of celecoxib related substances. Master's thesis, Peking Union Medical College, 2013.2013
2. Yang Hua, Xu Zhenping, He Mengting, He Yanping, Li Ling, Song Liudong. Synthesis of Mangiferin Metabolites and Xanthine Oxidase Inhibitory Activity. (Natural Products Research and Development) 2015-8.
3. Huang Zhenchi, Li Heng. Study on the inhibitory activity of xanthine oxidase from Aspergillus sinensis and Aloe. (Journal of Lingnan Normal University) 2017-06.
4. Taukoorah U, Mahomoodally M F. Crude Aloe vera Gel Shows Antioxidant Propensities and Inhibits Pancreatic Lipase and Glucose Movement In Vitro. Adv Pharmacol Sci. 2016; 2016:3720850. doi: 10.1155/2016/3720850. Epub 2016 Jan. 3.
5. Sarvaiya V N, Sadariya K A, Pancha P G, Thaker A M, Patel A C, Prajapati A S. Evaluation of antigout activity of Phyllanthus emblica fruit extracts on potassium oxonate-induced gout rat model. Vet World. 2015 October; 8(10):1230-6. doi: 10.14202/vetworld.2015.1230-1236. Epub 2015 Oct. 23.
6. Bernardo J, Ferreres F, Gil-Izquierdo Á, Videira R A, Valentáo P, Veiga F, Andrade P B. In vitro multimodal-effect of Trichilia catigua A. Juss. (Meliaceae) bark aqueous extract in CNS targets. J Ethnopharmacol. 2018 Jan. 30; 211:247-255. doi: 10.1016/j.jep.2017.09.039. Epub 2017 Sep. 29.
7. Belviranli M, Gabel H, Okudan N, Biiyiikba§ S. Effects of grape seed extract on oxidative stress and antioxidant defense markers in streptozotocin-induced diabetic rats. Turk J Med Sci. 2015; 45(3):489-95.
8. Wang Y, Zhu J X, Kong L D, Yang C, Cheng C H, Zhang X. Administration of procyanidins from grape seeds reduces serum uric acid levels and decreases hepatic xanthine dehydrogenase/oxidase activities in oxonate-treated mice. Basic Clin Pharmacol Toxicol. 2004 May; 94(5):232-7.
9. Zhao Ling, Chen Luyi, Li Heyu. Study on the inhibitory effect of a compound extract on xanthine oxidase. Food Research and Development, September 2015, Vol. 36 No. 17; 198-200, DOI: 10.3969/j.issn.1005.
10. Zhu C, Xu Y, Liu Z H, Wan X C, Li D X, Tai L L. The anti-hyperuricemic effect of epigallocatechin-3-gallate (EGCG) on hyperuricemic mice. Biomed Pharmacother. 2018 January; 97:168-173. doi: 10.1016/j.biopha.2017.10.013. Epub 2017 Nov. 6.
11. Zhu C, Tai L L, Wan X C, Li D X, Zhao Y Q, Xu Y. Comparative effects of green and black tea extracts on lowering serum uric acid in hyperuricemic mice. Pharm Biol. 2017 December; 55(1):2123-2128. doi: 10.1080/13880209.2017.1377736.
12. Chen G, Tan M L, Li K K, Leung P C, Ko C H. Green tea polyphenols decreases uric acid level through xanthine oxidase and renal urate transporters in hyperuricemic mice. J Ethnopharmacol. 2015 Dec. 4; 175:14-20. doi: 10.1016/j.jep.2015.08.043. Epub 2015 Sep. 3.
13. Pang M, Fang Y, Chen S, Zhu X, Shan C, Su J, Yu J, Li B, Yang Y, Chen B, Liang K, Hu H, Lv G. Gypenosides Inhibits Xanthine Oxidoreductase and Ameliorates Urate Excretion in Hyperuricemic Rats Induced by High Cholesterol and High Fat Food (Lipid Emulsion). Med Sci Monit. 2017 Mar. 4; 23:1129-1140.
14. Shi Shen, Chang Wei, Shang Xiaoyu, Wang Na, Li Sen, Zhang Zesheng. The inhibitory effects of several natural products on xanthine oxidase. Chinese Journal of Food Science, 2014, Issue 7.
15. Shen Qirong. Screening of Traditional Chinese Medicine Xanthine Oxidase Inhibitors and Study on Inhibition Kinetics. “Nanchang University” 2015. Master's degree thesis.
16. Zhao Shouhuan, Yang Hui, Shi Guanying Wang Xiaomin, Zhao Hongyuan, Wang Zhaogai. Response surface methodology to optimize the inhibitory effects of three natural products on xanthine oxidase. Science and Technology of Food Industry 2018, Issue 5, pages 230-234.
17. Feng Wenci. Identification of five kinds of herbal tea hot water extracts that inhibit xanthine oxidase activity and their enzyme kinetics. National Taiwan Ocean University, Master's thesis, 2017-06-29.
18. Hatano T, Yasuhara T, Fukuda T, Noro T, Okuda T. Phenolic constituents of licorice. II. Structures of licopyranocoumarin, licoarylcoumarin and glisoflavone, and inhibitory effects of licorice phenolics on xanthine oxidase. Chem Pharm Bull (Tokyo). 1989 November; 37(11):3005-9.
19. Zarrelli A, Romanucci V, Tuccillo C, Federico A, Loguercio C, Gravante R, Di Fabio G. New silibinin glyco-conjugates: synthesis and evaluation of antioxidant properties. Bioorg Med Chem Lett. 2014 Nov. 15; 24(22):5147-9. doi: 10.1016/j.bmc1.2014.10.023. Epub 2014 Oct. 14.
20, Liang Wenjuan, He Jinsong, Tian Yang, Zhao Dekun, Wang Hongling. Moringa oleifera leaf extract reduces uric acid levels in mice with hyperuricemia and its mechanism. “Anhui Agricultural Sciences” 2017, Issue 17, pages 108-109.
21. De Marino S, Festa C, Zollo F, Nini A, Antenucci L, Raimo G, lorizzi M. Antioxidant activity and chemical components as potential anticancer agents in the olive leaf (Olea europaea L. cv Leccino.) decoction. Anticancer Agents Med Chem. 2014; 14(10):1376-85.
22. Flemmig J, Kuchta K, Arnhold J, Rauwald H W. Olea europaea leaf (Ph. Eur.) extract as well as several of its isolated phenolics inhibit the gout-related enzyme xanthine oxidase. Phytomedicine. 2011 May 15; 18(7):561-6. doi: 10.1016/j.phymed.2010.10.021. Epub 2010 Dec. 8.
23. Azmi S M N, Jamal P, Amid A. Xanthine oxidase inhibitory activity from potential Malaysian medicinal plant as remedies for gout. International Food Research Journal, 2012; 19(1), 159-65.
24. Zhang Xue, Zhou Ying, Guan Jing, Yan Shouqun, Ping Tingting. Inhibition of xanthine oxidase by extracts of five Chinese medicinal materials. Journal of Mountain Agriculture and Biology, 2015, Issue 4, 51-54.5
25. Moini H, Guo Q, Packer L. Enzyme inhibition and protein-binding action of the procyanidin-rich french maritime pine bark extract, pycnogenol: effect on xanthine oxidase. J Agric Food Chem. 2000 November; 48(11):5630-9.
26. Les F, Prieto J M, Arbonés-Mainar J M, Valero M S, López V. Bioactive properties of commercialised pomegranate (Punica granatum) juice: antioxidant, antiproliferative and enzyme inhibiting activities. Food Funct. 2015 June; 6(6):2049-57. doi: 10.1039/c5fo00426h.
27. Sestili P, Martinelli C, Ricci D, Fraternale D, Bucchini A, Giamperi L, Curcio R, Piccoli G, Stocchi V. Cytoprotective effect of preparations from various parts of Punica granatum L. fruits in oxidatively injured mammalian cells in comparison with their antioxidant capacity in cell free systems. Pharmacol Res. 2007 July; 56(1):18-26. Epub 2007 Feb. 20.
28. Rummun N, Somanah J, Ramsaha S, Bahorun T, Neergheen-Bhujun V S. Bioactivity of Nonedible Parts of Punica granatum L.: A Potential Source of Functional Ingredients. Int J Food Sci. 2013; 2013:602312. doi: 10.1155/2013/602312. Epub 2013 Jul. 8.
29. Namuslu M, Kocaoglu H, Celik H T, Avci A, Devrim E, Genc Y, Gocmen E, Erguder I B, Durak I. Effects of aqueous soybean, mistletoe and red clover extracts on activities of adenosine deaminase and xanthine oxidase enzyme. Bratisl Lek Listy. 2014; 115(6):367-71.
30. Satyal P, Jones T H, Lopez E M, McFeeters R L, Ali N A, Mansi I, Al-Kaf A G, Setzer W N. Chemotypic Characterization and Biological Activity of Rosmarinus officinalis. Foods. 2017 Mar. 5; 6(3). pii: E20. doi: 10.3390/foods6030020.
31. Shang Yanjun, Huang Caiguo, Jiang Sanhao, Zhu Dayuan, Wei Shanjian, Jiao Binghua. Inhibition of rosmarinic acid on xanthine oxidase. Journal of Second Military Medical University; Volume 27, Issue 2 (2006/02/20), P189-191.
32. Liu Lu. Inhibitory activity of xanthine oxidase in vitro from Chinese herbal medicine and its compound beverage. South China University of Technology, 2014, Issue 05, Master's degree thesis.
33. Havlik J, Gonzalez de la Huebra R, Hejtmankova K, Fernandez J, Simonova J, Melich M, Rada V. Xanthine oxidase inhibitory properties of Czech medicinal plants. J Ethnopharmacol. 2010 Nov. 11; 132(2):461-5. doi: 10.1016/j.jep.2010.08.044. Epub 2010 Aug. 26.
34. Lu J M, Yao Q, Chen C. 3,4-Dihydroxy-5-nitrobenzaldehyde (DHNB) is a potent inhibitor of xanthine oxidase: a potential therapeutic agent for treatment of hyperuricemia and gout. Biochem Pharmacol. 2013 Nov. 1; 86(9):1328-37.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1.-121. (canceled)

122. An isolated composition comprising Japanese thistle extract and Chinese mint extract.

123. The composition of claim 122, further comprising one or more of Siberian Ginseng Extract, Citron (Fruit) Extract, Butcher's Broom Extract Powder, Yohimbe bark Extract Powder, Rugose Rose (Flower Bud) Extract, Oolong Tea (leaf) Extract Powder.

124. The composition of claim 122, further comprising Folic Acid or a functionally active derivative thereof.

125. The composition of claim 122, further comprising Milk Thistle Extract Powder, Amla Fruit Powder, Rhodiola rosea linn Extract, Grape Seed Extract, Pomegranate Extract, Green Tea Pure Extract, Pine Bark Extract, St. John's Wort Extract, Lesser Galangal Extract, Honeysuckle (Flower Bud) Extract, African Mango seed Extract Powder, Clove (Flower Bud) Extract, or a combination thereof.

126. The composition of claim 122, further comprising Sacred Lotus (Seed) Extract, Indian Madder (Root and Rhizome) Extract, Senna Leaf Extract, Cyathula Extract, Caralluma bark (wild) Extract Powder, Muira Puama Bark Extract Powder, Euryale (Seed) Extract, Wheat (Immature Fruit) Extract, Houttuynia (Aboveground Parts) Extract, Rice Bean Extract, or a combination thereof.

127. The composition of claim 122, further comprising Vitamin D3.

128. The composition of claim 122, further defined as comprising Japanese thistle extract, Chinese mint extract, Folic Acid, Amla Fruit Powder, Milk Thistle Extract Powder, vitamin D3, and Rhodiola Rosea Linn Extract.

129. The composition of claim 122, wherein the Japanese thistle extract or Chinese mint extract is a DMSO-soluble extract.

130. The composition of claim 122, wherein the composition is formulated as a food supplement or is formulated in a pharmaceutically acceptable excipient.

131. The composition of claim 122, wherein the composition is a capsule, tablet, pill, film, lozenge, powder, or combination thereof.

132. The composition of claim 122, wherein the composition is in the form of a solid, liquid, or gel.

133. The composition of claim 122, wherein the composition further comprises febuxostat, Allopurinol, or both.

134. A method of treating or preventing a hyperuricemic condition or a medical condition related to oxidative stress in an individual, comprising the step of providing to the individual an effective amount of a composition of claim 122.

135. The method of claim 134, wherein the hyperuricemic condition is asymptomatic.

136. The method of claim 134, wherein the hyperuricemic condition is symptomatic.

137. The method of claim 134, wherein the hyperuricemic condition is gout, hypertension, atherosclerosis, coronary artery disease, heart failure, left ventricular hypertrophy, atrial fibrillation, periphery artery disease, vascular restenosis, vascular thrombosis, stroke, diabetes, insulin resistance, metabolic syndrome, chronic kidney disease, psoriatic arthritis, micro-albuminuria, erectile dysfunction, preeclampsia, cancers, immune disorders, tumor lysis syndrome, or an inflammatory disease.

138. The method of claim 134, wherein the individual is provided an effective amount of febuxostat, Allopurinol, or both.
